(19) United States
(12) Patent Application Publication
(10) Pub. No.: US 2022/0243189 A1
(43) Pub. Date: Aug. 4, 2022

(54) PROCOAGULANT COMPOUNDS

(71) Applicant: Bioverativ Therapeutics Inc., Waltham, MA (US)

(72) Inventors: Vu Phong HONG, Cambridge, MA (US); Adam R. MEZO, Carmel, IN (US); Joe SALAS, Wayland, MA (US); Robert PETERS, West Roxbury, MA (US)

(21) Appl. No.: 17/577,684

(22) Filed: Jan. 18, 2022

Related U.S. Application Data
(63) Continuation of application No. 16/357,153, filed on Mar. 18, 2019, now Pat. No. 11,261,437.
Application No. 16/357,153 is a division of application No. 14/406,163, filed on Dec. 5, 2014, now Pat. No. 10,287,564.
(60) Provisional application No. 61/657,688, filed on Jun. 8, 2012.

(51) Int. Cl. C12N 9/64; A61K 38/02; A61K 38/36; A61K 38/48

(52) U.S. Cl. CPC C12N 9/6437; A61K 38/02; C12N 9/6432; A61K 38/4846; A61K 38/36

(57) ABSTRACT

The present disclosure provides protease-activatable procoagulant compounds comprising a procoagulant polypeptide, e.g., a procoagulant peptide and/or clotting factor, and a linker comprising a protease-cleavable substrate (e.g., a synthetic thrombin substrate) and a self-immolative spacer (e.g., p-amino benzyl carbamate). Upon cleavage of the protease-cleavable substrate by a protease (e.g., thrombin), the self-immolative spacer cleaves itself from the procoagulant polypeptide such that the polypeptide is in an underivatized and active form. Also provided are pharmaceutical compositions, methods for treating bleeding disorders using the disclosed compounds, methods of enhancing in vivo efficacy of procoagulant polypeptides, methods of increasing the efficacy of proteolytic cleavage of compounds comprising procoagulant polypeptides, methods of activating procoagulant polypeptides, and methods of releasing a procoagulant polypeptide from a heterologous moiety such as PEG.

### RELATED APPLICATIONS

This application is a continuation of U.S. patent application Ser. No. 16/357,153, filed Mar. 18, 2019, which is a division of U.S. patent application Ser. No. 14/406,163, filed Dec. 5, 2014, now U.S. Pat. No. 10,287,564, which is a 35 U.S.C. §371 filing of International Patent Application No. PCT/US2013/044841, filed Jun. 7, 2013, which claims priority to U.S. Provisional Patent Application Ser. Nos. 61/800,626, filed Mar. 15, 2013, and 61/657,688, filed Jun. 8, 2012, the entire disclosures of which are hereby incorporated herein by reference.

### BACKGROUND

### Field of the Disclosure

The present invention relates to procoagulant compounds useful for the treatment of bleeding diseases or disorders.

### Background

The blood coagulation pathway, in part, involves the formation of an enzymatic complex of Factor VIIIa (FVIIIa) and Factor IXa (FIXa) (Xase complex) on the surface of platelets. FIXa is a serine protease with relatively weak catalytic activity without its cofactor FVIIIa. The Xase complex cleaves Factor X (FX) into Factor Xa (FXa), which in turn interacts with Factor Va (FVa) to cleave prothrombin and generate thrombin. Hemophilia A is a bleeding disorder caused by mutations and/or deletions in the factor VIII (FVIII) gene resulting in a deficiency of FVIII activity (Peyvandi et al. 2006). Hemophilia B (also known as Christmas disease) is one of the most common inherited bleeding disorders in the world. It results in decreased in vivo and in vitro blood clotting activity and requires extensive medical monitoring throughout the life of the affected individual.

Treatment of hemophilia is by replacement therapy targeting restoration of clotting activity. There are plasma-derived and recombinant clotting factor products available to treat bleeding episodes on-demand or to prevent bleeding episodes from occurring by treating prophylactically. Based on the half-life of these products, treatment regimens require frequent intravenous administration. Such frequent administration is painful and inconvenient. Strategies to extend the half-life of clotting factors include pegylation (Rostin J, et al., Bioconj. Chem. 2000; 11:387-96), glycopegylation (Stennicke HR, et al., Thromb. Haemost. 2008; 100:920-8), formulation with pegylated liposomes (Spira J, et al., Blood 2006; 108:3668-3673, Pan J, et al., Blood 2009; 114:2802-2811) and conjugation with albumin (Schulte S., Thromb. Res. 2008; 122 Suppl 4:S14-9). However, modification of coagulation factors and procoagulant peptides with half-life extending moieties (e.g., PEG) and other similar strategies to extend their half-lives can lead to compromised activity. In order to rescue their activity, a cleavable linker can be inserted between the protein or peptide of interest and its modifier. The chosen cleavable linker must be cleaved efficiently and rapidly by a protease, for example, a protease involved in the coagulation cascade. Thrombin being the activator of many clotting factors is the most popular choice. However, all known substrate sequences composed of natural amino acids (e.g., LVPR, ALRPR (SEQ ID NO: 7), etc.) are not optimal substrates. Furthermore, covalent binding of the cleavable linker to a coagulation factors or procoagulant peptide can result in steric hindrances (e.g., due to the presence of amino acids such as such as proline, isoleucine or arginine C-terminal to the cleavage site) that can prevent an efficient enzymatic cleavage reaction.

### BRIEF SUMMARY

The present disclosure provides procoagulant compounds comprising a protease-cleavable substrate (e.g., a synthetic thrombin substrate) and a self-immolative spacer (e.g., PABC) linked to a procoagulant polypeptide, e.g., a clotting factor or a procoagulant peptide. Accordingly, in some embodiments, the present disclosure provides A procoagulant compound having a formula:

(Het2)-(Pep2)-(Het1)-(L)-Zy-Bx-Pep1 (Formula I)

wherein,

Het1 is a first heterologous molecule, which is either absent or present;

Het2 is a second heterologous molecule, which is either absent or present;

L is a linker, which is either absent or present;

Zy is a protease-cleavable substrate;

Bx is a self-immolative spacer;

Pep1 is a polypeptide; and,

Pep2 is a polypeptide, which is either absent or present;

wherein, Pep1 or Pep2 comprises a clotting factor or a fragment thereof, or a synthetic procoagulant peptide.

In some embodiments, the self-immolative spacer in the procoagulant compound of the invention undergoes 1,4 elimination after the enzymatic cleavage of the protease-cleavable substrate. In some embodiments, the self-immolative spacer in the procoagulant compound of the invention undergoes 1,6 elimination after the enzymatic cleavage of the protease-cleavable substrate. In some embodiments, the self-immolative spacer is a p-amino benzyl carbamate (PABC), a p-amino benzyl ether (PABE), or a p-amino benzyl carbonate. In certain embodiments, the self-immolative spacer comprises an aromatic group. In some embodiments, the aromatic group is selected from the group consisting of benzyl, cinnamyl, naphthyl, and biphenyl. In some embodiments, the aromatic group is heterocyclic. In other embodiments, the aromatic group comprises at least one substituent. In some embodiments, at least one substituent is selected from F, Cl, I, Br, OH, methyl, methoxy, NO₂, NH₂, NO³⁺, NHCOCH₃, N(CH₃)₂, NHCOCF₃, alkyl, haloalkyl, C₁-C₈ alkylhalide, carboxylate, sulfate, sulfamate, sulfonate, or any combinations thereof. In other embodiments, at least one C in the aromatic group is substituted with N, O, or C—R₁, wherein R₁ is independently selected from H, F, Cl, I, Br, OH, methyl, methoxy, NO₂, NH₂, NO³⁺, NHCOCH₃, N(CH₃)₂, NHCOCF₃, alkyl, haloalkyl, C₁-C₈ alkylhalide, carboxylate, sulfate, sulfamate, and sulfonate.

In some embodiments, the protease-cleavable substrate in the procoagulant compound of the invention comprises a coagulation cascade protease substrate. In some embodiments, the coagulation cascade protease is selected from thrombin, thromboplastin, Factor Va, Factor VIla, Factor VIIIa, Factor IXa, Factor Xa, Factor XIa, Factor XIIa, or any combinations thereof. In other embodiments, the coagulation cascade protease substrate is a thrombin substrate. In some embodiments, the thrombin substrate is a synthetic thrombin substrate. In other embodiments, the synthetic thrombin substrate comprises the sequence of D-Phe-Pip-Arg. In some embodiments, the thrombin substrate is selected from D-Phe-Pro-Arg, D-Ala-Leu-Val-Pro-Arg (SEQ ID NO: 17), Ala-Leu-Val-Pro-Arg (SEQ ID NO: 17), Leu-Val-Pro-Arg (SEQ ID NO: 18), or Ala-Leu-Arg-Pro-Arg (SEQ ID NO: 90).

In some embodiments, the protease-cleavable substrate comprises a cleavage site for a protease selected from neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases, enamelysin, tumor necrosis factor α-converting enzyme, insulysin, nardilysin, mitochondrial processing peptidase, magnolysin, dactylysin-like metalloproteases, neutrophil collagenase, matrix metallopeptidases, membrane-type matrix metalloproteinases, SP2 endopeptidase, prostate specific antigen (PSA), plasmin, urokinase, human fibroblast activation protein (FAPα), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose- binding protein-associated serine protease, coagulation factors, thrombin, protein c, u and t-type plasminogen activator, cathepsin G, hepsin, prostasin, hepatocyte growth factor- activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin D, pepsins, chymosyn, gastricsin, renin, yapsin and/or memapsins, Prostate-Specific antigen (PSA), or any combinations thereof.

In some embodiments, Pep1 and Pep2 are different. In other embodiments, Pep1 and Pep2 are the same. In some embodiments, Pep1 is a clotting factor or a fragment thereof In other embodiments, wherein Pep2 is a clotting factor or a fragment thereof. In certain embodiments, both Pep1 and Pep2 are clotting factors or fragments thereof.

In some embodiments, Pep1 is a heavy chain of a clotting factor and Pep2 is a light chain of the clotting factor. In other embodiments, the clotting factor is selected from FVII, FVIIa, FVIII, FIX, FX, FXa, vWF, or any combinations thereof. In other embodiments, Pep1 is a synthetic procoagulant peptide. In other embodiments, Pep2 is a synthetic procoagulant peptide. In some embodiments, both Pep1 and Pep2 are synthetic procoagulant peptides.

In some embodiments, the linker (L) is a peptide linker. In some embodiments, the peptide linker comprises at least two amino, at least three, at least four, at least five, at least 10, at least 20, at least 30, at least 40, at least 50, at least 60, at least 70, at least 80, at least 90, or at least 100 amino acids. In other embodiments, the peptide linker comprises at least 200, at least 300, at least 400, at least 500, at least 600, at least 700, at least 800, at least 900, or at least 1,000 amino acids. In some embodiments, the peptide linker comprises a peptide having the formula [(Gly)ₓ-Ser_{y}]_{z} where x is from 1 to 4, y is 0 or 1, and z is from 1 to 50. In some embodiments, the linker (L) comprises a non-peptide linker.

In some embodiments, the linker (L) consists of a non-peptide linker. In other embodiments, the non-peptide linker is selected from MC, MP, MPEG, SMCC, MBS, SMPT, LC-SPDP, SMPB, or any combinations thereof

In some embodiments, the heterologous moiety comprises a half-life extending moiety. In other embodiments, the half-life extending moiety is a low-complexity polypeptide. In other embodiments, the half-life extending moiety comprises albumin, albumin binding polypeptide or fatty acid, Fc, transferrin, PAS, the C-terminal peptide (CTP) of the β subunit of human chorionic gonadotropin, polyethylene glycol (PEG), hydroxyethyl starch (HES), albumin-binding small molecules, vWF, XTEN, or any combinations thereof. In some embodiments, the half-life extending moiety comprises a clearance receptor or fragment thereof which blocks binding of the procoagulant compound to a clearance receptor. In some embodiments, the clearance receptor is LRP1.

In some embodiments, the heterologous moiety comprises a peptide or a polypeptide which enables visualization or localization of the procoagulant compound or a fragment thereof. In some embodiments, the visualization or localization is enabled in vitro, in vivo, ex vivo or any combination thereof. In some embodiments, the peptide or the polypeptide which enables visualization or localization is selected from a biotin acceptor peptide, a lipoic acid acceptor peptide, a fluorescent protein, a cysteine-containing peptide for ligation of a biarsenical dye or for conjugating metastable technetium, a peptide for conjugating europium clathrates for fluorescence resonance energy transfer (FRET)-based proximity assays, or any combination thereof.

In some embodiments, the fluorescent protein is selected from GFP, RFP, YFP, EGFP, EYFP, or any combination thereof. In some embodiments, the biarsenical dye is 4′,5′-bis(1,3,2-dithioarsolan-2-yl)fluorescein (FlAsH). In some embodiments, the biotin acceptor peptide facilitates conjugation of avidin- and streptavidin-based reagents. In some embodiments, the lipoic acid acceptor peptide facilitates conjugation of thiol-reactive probes to bound lipoic acid or direct ligation of fluorescent lipoic acid analogs.

In some embodiments, the heterologous moiety comprises a non-peptidic active agent. In some embodiments, the non-peptidic active agent is a procoagulant molecule. In some embodiments, the non-peptidic active agent is a small molecule drug. In some embodiments, the heterologous moiety comprises a targeting or ligand binding moiety. In some embodiments, the heterologous moiety comprises an anchor or scaffolding molecule. In some embodiments, the anchor or scaffolding molecule is a lipid, a carbohydrate, or a sulfhydryl group.

In some embodiments, the procoagulant compound of the invention comprises the formula Het-L-Zy-Bx-Pep1, wherein:
Het is cysteine,
L is a peptide linker,
Zy is a synthetic thrombin substrate,
Bx is the self-immolative spacer, and
Pep1 is procoagulant peptide.

In some embodiments, the peptide linker comprises a GGGG amino acid sequence, the synthetic thrombin substrate comprises the sequence DPhe-Pip-Arg, the self-immolative spacer is PABC, and the procoagulant peptide comprises the sequence:

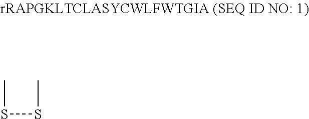

In some embodiments, the procoagulant compound of the invention comprises the formula Zy-Bx-Pep1, wherein: Zy is a synthetic thrombin substrate, Bx is a self-immolative spacer, and Pep1 is a clotting factor. In some embodiments, the synthetic thrombin substrate comprises the sequence DPhe-Pip-Arg, the self-immolative spacer is PABC, and the clotting factor is Factor Xa or FVIIa.

In some embodiments, the procoagulant activity of the procoagulant compounds of the invention is measured using a method selected from an activated partial thromboplastin time (aPTT) assay, a modified activated partial thromboplastin time (aPTT*) assay, a thrombin generation assay (TGA), and a ROTEM assay.

The present disclosure also provides a pharmaceutical composition comprising a procoagulant compound of the invention, and a pharmaceutically acceptable carrier.

Also provided is a method for treating, ameliorating, or preventing a bleeding disease or disorder in a subject, comprising administering to the subject an effective amount of a procoagulant compound of the invention or a pharmaceutical composition comprising the procoagulant compound of the invention. In some embodiments, the bleeding disease or disorder is caused by a blood coagulation disorder. In some embodiments, the blood coagulation disorder is selected from hemophilia and von Willebrand disease (vWD). In some embodiments, the blood coagulation disorder is hemophilia A or hemophilia B. In some embodiments, the bleeding disease or disorder is selected from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

The present disclosure also provides a method of treating, ameliorating, or preventing a deficiency in at least one blood coagulation factor in mammalian subject, wherein the blood coagulation factor is selected from FV, FVII, FVIIa, FVIII, FIX, FX, FXI, and vWF, the method comprising administering to the subject an effective amount of the procoagulant compound of the invention or a pharmaceutical composition comprising the procoagulant compound. In some embodiments, the subject is a human subject.

In some embodiments, the procoagulant compound of the invention or a pharmaceutical formulation comprising the procoagulant compound of the invention are used for treating a subject having a blood coagulation disorder. In some embodiments, the subject is a human subject. In some embodiments, the procoagulant compound of the invention or a pharmaceutical formulation comprising the procoagulant compound of the invention are used for the manufacture of a medicament for the treatment, prevention, or amelioration of a blood coagulation disorder.

Also provided in the present disclosure is a method for making the procoagulant compound of the invention comprising using solid-phase peptide synthesis. In some embodiments, the method uses orthogonal solid-phase peptide synthesis.

The present disclosure also provides a method of enhancing in vivo efficacy of a procoagulant polypeptide comprising coupling the polypeptide to a self-immolative spacer, wherein said self-immolative spacer is coupled to a protease-cleavable substrate moiety. In some embodiments, the self-immolative spacer comprises a PABC group. In some embodiments, the protease-cleavable substrate moiety comprises a synthetic thrombin substrate. In some embodiments, the procoagulant polypeptide is a clotting factor or a procoagulant peptide. In some embodiments, the procoagulant peptide is synthetic.

The present disclosure also provides a method of increasing the efficacy of the cleavage of a protease substrate operably linked to a procoagulant peptide or clotting factor comprising conjugating a self-immolative linker to said procoagulant polypeptide, wherein said self-immolative linker is interposed between the protease substrate and the procoagulant peptide or clotting factor. Also disclosed is a method of activating a procoagulant peptide comprising contacting a procoagulant compound of the invention with a protease specific for the protease-cleavable substrate moiety in said procoagulant compound, wherein the activated procoagulant peptide is released upon proteolytic cleavage of the protease-cleavable substrate moiety.

Also provided is a method of activating a clotting factor comprising contacting a procoagulant compound of the invention with a protease specific for the protease-cleavable substrate moiety in said procoagulant compound, wherein the activated clotting factor is released upon proteolytic cleavage of the protease-cleavable substrate moiety. The instant disclosure also provides a method of releasing a procoagulant peptide from a heterologous moiety comprising contacting a procoagulant compound of the invention with a protease specific for the protease-cleavable substrate in said procoagulant compound, wherein the activated procoagulant polypeptide is released upon proteolytic cleavage of the protease-cleavable substrate.

The present disclosure also provides a method of releasing a clotting factor from a heterologous moiety comprising contacting a procoagulant compound of the invention with a protease specific for the protease-cleavable substrate in said procoagulant compound, wherein the activated clotting factor is released upon proteolytic cleavage of the protease-cleavable substrate. In some embodiments, the procoagulant compound is cleaved by a protease specific for the protease-cleavable substrate moiety at least 2-fold, at least 3-fold, at least 4-fold, at least 5-fold, at least 6-fold, at least 7-fold, at least 8-fold, at least 9-fold or at least 10-fold-faster when compared to a reference procoagulant compound with the same sequence but without a self-immolative linker. In some embodiments, the procoagulant compound is cleaved by a protease specific for the protease-cleavable substrate moiety at least 20-fold, at least 30-fold, at least 40-fold, at least 50-fold, at least 60-fold, at least 70-fold, at least 80-fold, at least 90-fold or at least 100-fold-faster when compared to a reference procoagulant compound with the same sequence but without a self-immolative linker.

In some embodiments, the procoagulant compound of the invention comprises a self-immolative spacer comprising an exosite binding peptide.

### BRIEF DESCRIPTION OF THE DRAWINGS/FIGURES

FIG. 1A shows the general organization of a protease-activatable procoagulant compound of the invention. Het2, Pep2, Het1 and L are independently optional components. Pep1 and Pep2 are polypeptides, at least one of which is a clotting factor or a procoagulant peptide. Het1 and Het are heterologous moieties. L is a linker. Additional linkers can connect the different moieties; for example, a linker could be located between Pep2 and Het1 (as shown in the diagram). Additional protease cleavable substrate and self-immolative spacer groups can be inserted at the N-terminus of other moieties such as polypeptides or heterologous moieties. The diagram shows the optional insertion of such a group at the N-terminus of Pep2.

FIG. 1B is a representation of an exemplary procoagulant compound of the invention comprising a protease cleavable substrate (Aa₁Aa₂Aa₃Aa₄), a self-immolative spacer and a protein of interest (POI; e.g., a clotting factor or procoagulant peptide); illustrating the fragmentation of the compound and the release of the peptide or protein of interest after proteolytic cleavage of the cleavable substrate and 1,6 spontaneous fragmentation.

FIG. 2 is a representation of an alternative exemplary protease-activatable procoagulant compound of the invention which comprises an exosite binding peptide (M). The diagram illustrates the release of the peptide or protein of interest (POI; e.g., a clotting factor or procoagulant peptide) and the exosite binding peptide after proteolytic cleavage of a cleavable substrate (Aa₁Aa₂Aa₃Aa₄) and 1,6 spontaneous fragmentation.

FIG. 3 and FIG. 4 show the general synthesis scheme for protease-activatable procoagulant compound of the invention. The diagrams correspond to the synthesis of Compound 7. FIG. 3 shows the reactions leading to the synthesis of the compound comprising the protease cleavable substrate (thrombin substrate) and the self-immolative spacer (PABC). FIG. 4 shows the conjugation of the substrate/PABC compound to the synthetic procoagulant peptide and the deprotection of the resulting product to yield Compound 7.

FIG. 5 presents a schematic representation of the cleavage of Compound 7 by thrombin.

FIG. 6 shows the kinetics of the cleavage of Compound 7 by thrombin.

FIG. 7 shows the cleavage of Compound 7 during the course of a TGA assay.

FIG. 8 shows the release kinetics of the peptide IVGGQE (SEQ ID NO: 85), which corresponds to the six N-terminal amino acid residues of the heavy chain of the FXa clotting factor, from different procoagulant compounds (Compounds 1, 2, and 3) following treatment with 14 nM thrombin.

FIG. 9 shows the release kinetics of the peptide IVGGQE (SEQ ID NO: 85), which corresponds to the six N-terminal amino acid residues of the heavy chain of the FXa clotting factor, from different procoagulant compounds (Compounds 1, 4, 5 and 6) following treatment with 1.4 nM thrombin.

FIG. 10 shows the natural processing of factor X to yield activated factor X (FXa).

FIG. 11 is a representation of exemplary procoagulant compounds of the invention comprising FXa clotting factor.

FIG. 12 shows the natural processing of factor VII to yield activated factor VII (FVIIa).

FIG. 13 is a representation of exemplary procoagulant compounds of the invention comprising FVIIa clotting factor.

FIGS. 14A-14C show a flow diagram of a cleavable polypeptide, FVII-186 (FIG. 14A) that can be processed by a proprotein convertase (e.g., PACE) to a processed cleavable polypeptide (FIG. 14B). FIG. 14A shows a cleavable polypeptide comprising FVIILC (FVII light chain)—Proprotein Convertase Processing Site by a proprotein convertase (e.g., PACE processing site, e.g., 2X(RKR) (SEQ ID NO: 88))—Linker1—SUMO—Truncated FVIIHC (FVII heavy chain without IVGGKV (SEQ ID NO: 83) at the N-terminus)—Linker2—Fc Region2—Linker3—Fc Region2. FIG. 14B shows a schematic diagram of a cleavable polypeptide that has been processed by PACE. The processed cleavable polypeptide comprises two polypeptide chains, the first chain comprising FVIILC linked to the Proprotein Convertase processing site and the second chain comprising Linker1—SUMO—Truncated FVIIHC (FVII heavy chain without IVGGKV (SEQ ID NO: 83) at the N-terminus)—Linker2—Fc Region1—Linker3—Fc Region2. FIG. 14C demonstrates non-reduced (lane 1) or reduced (lane 2) SDS-PAGE, showing the above constructs and chains. (—) indicates a peptide bond.

FIGS. 15A-15D show a flow diagram of (i) FVII-186 cleavage by a SUMO protease (FIG. 15B) and (ii) its fusion to a thioester peptide (FIG. 14C). FIG. 15A is identical to the construct in FIG. 14B. FIG. 15B shows that, after FVII-186 is cleaved by a SUMO protease, the resulting cleaved polypeptide construct comprises two chains, the first chain comprising FVIILC and Proprotein Convertase Site and the second chain comprising Truncated FVIIHC (FVII heavy chain without IVGGKV (SEQ ID NO: 83) at the N-terminus)—Linker2—Fc Region1—Linker3—Fc Region2. The first chain and the second chain are linked by a disulfide bond. FIG. 15C shows that after the cleaved polypeptide construct in FIG. 15B is ligated with a thioester peptide (Biotin-Pra-GGGG-D-Phe-Pip-Arg-PABC-IVGGKV-COSBn (SEQ ID NO: 79)), the resulting construct comprises two polypeptide chains, the first chain comprising FVIILC and Proprotein Convertase Processing Site and the second chain comprising Thrombin cleavage site—FVIIHC (FVII heavy chain)—Linker2—Fc Region1—Linker3—Fc Region2 (TA-FVII-186). FIG. 15D shows reducing SDS-PAGE indicating the constructs and chains: lane 1 shows marker; lane 2 shows FVII-186; lane 3 shows FVII-186 with SUMO protease reaction; lane 3 shows FVII-186 with SUMO protease reaction and conjugation with a positive control peptide; and lane 5 shows FVII-186 with SUMO protease reaction and conjugation with PABC peptide. (—) indicates a peptide bond.

FIG. 16 shows FVIIa chromogenic assay after thrombin activation of TA-FVII-186. X axis indicates time (min), and Y axis indicates Absorbance (A405) measurement for FVIIa activity. (x) shows FVIIa activity of a mixture of thrombin and hirudin. (□) indicates FVIIa activity of a mixture of FVII-186, thrombin, and hirudin. (◯) indicates FVIIa activity of a mixture of TA-FVII-186, thrombin, and hirudin.

FIGS. 17A-17C show a flow diagram of FX-011 expression by a proprotein convertease (e.g., PACE). FIG. 17A shows a cleavable polypeptide construct comprising FXLC (Factor X light chain)—AP (activation peptide)—Proprotein Convertase Processing Sitel (e.g., 2X(RKR) SEQ ID NO: 88))—Truncated FXHC (Factor X heavy chain without six amino acids (i.e., IVGGQE (SEQ ID NO: 85)) at the N-terminus)—Fc Regionl—Proprotein Convertase Processing Site2 (e.g., RRRR (SEQ ID NO: 89))—Linker—Fc Region2. When the cleavable construct of FIG. 16A is expressed, the initial construct can be processed by a proprotein convertase (e.g., PACE) to three polypeptide chains construct, the first chain comprising FXLC, the second chain comprising Truncated FXHC (Factor X heavy chain without six amino acids (i.e., IVGGQE (SEQ ID NO: 85) at the N-terminus)—Fc Region1—Proprotein Convertase Processing Site2, and the third chain comprising Fc Region2. FIG. 17C shows reduced (lane 2) and non-reduced (lane 3) SDS-PAGE showing the chains and constructs. (—) indicates a peptide bond.

FIGS. 18A-18C show a flow diagram of thrombin-cleavable Factor X molecule synthesis. FIG. 18A construct, identical to FIG. 17B contruct (FX-011), is incubated with a thioester peptide (GG-D-Phe-Pip-Arg-PABC-IVGGQE-COSBn (SEQ ID NO: 80)). The resulting construct (TA-FX-011) comprises three chains, the first chain comprising FXLC, the second chain comprising Thrombin Cleavage Site (D-Phe-Pip-Arg-PABC)—FXHC—Fc Region1—Proprotein Convertase Processing Site, and the third chain comprising Fc Region 2. The first chain and the second chain are bound by a disulfide bond, and the second and the third chains are bound by two disulfide bonds. FIG. 18C shows the constructs and chains in SDS-PAGE. (—) indicates a peptide bond.

FIG. 19 shows FXa chromogenic assay after thrombin activation of TA-FX-011. X axis indicates time (min), and Y axis indicates Absorbance (A405) measurement for FXa activity. (x) shows FXa activity of a mixture of thrombin and hirudin. (-) indicates FXa activity of a mixture of FX-011, thrombin, and hirudin. (o) indicates FXa activity of a mixture of TA-FXa-011, thrombin, and hirudin.

FIGS. 20A-20C show a flow diagram of a cleavable FX polypeptide construct (FX-012). FIG. 20A shows a cleavable polypeptide comprising FXLC-SUMO-Truncated FXHC (Factor X heavy chain without six amino acids, i.e., IVGGQE (SEQ ID NO: 85) at the N-terminus)—Fc Region1—Proprotein Convertase Processing Site1-Linker-Proprotein Converase Processing Site2—Fc Region2. The FIG. 20A construct can be intracellularly processed by a proprotein convertase (e.g., PACE) to result in the FIG. 20B construct. FIG. 20B shows a processed cleavable FX polypeptide comprising three chains (FX-012), the first chain comprising FXLC, the second chain comprising SUMO—Truncated FXHC—Fc Regionl-Proprotein Convertase Processing Sitel, and the third chain comprising Fc Region2. The first chain and the second chain are bound by a disulfide bond. The second chain and the third chain are bound by two disulfide bonds.

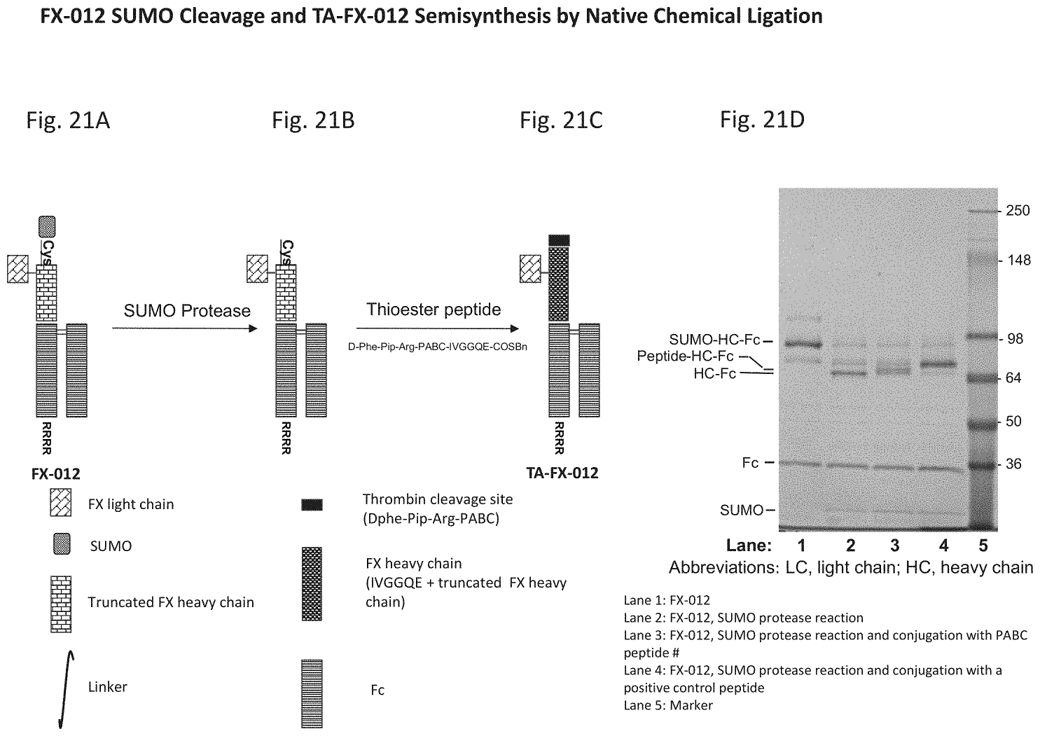

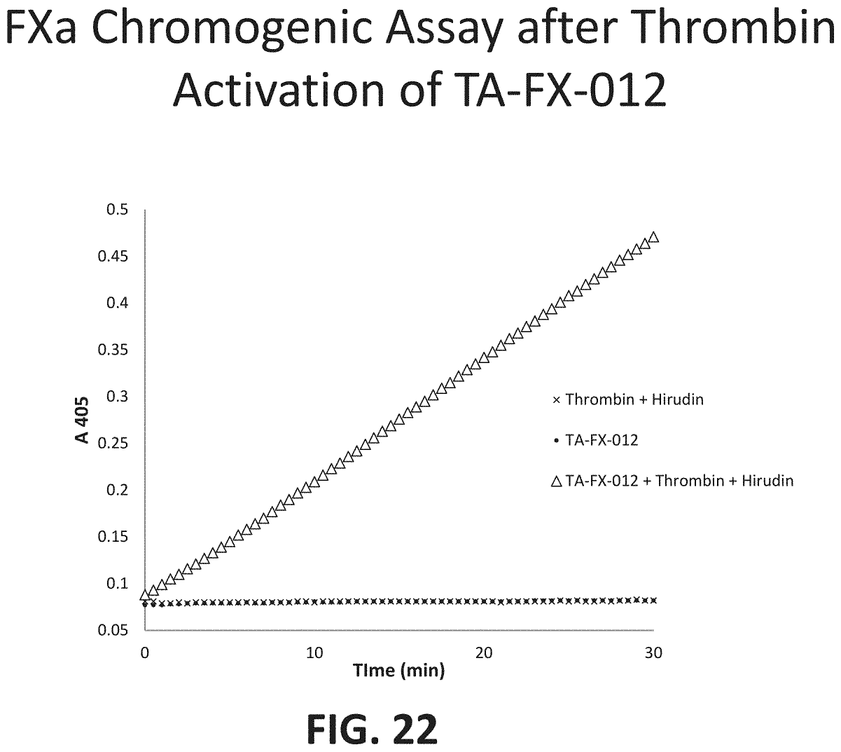

### FIG. 20C Shows a Western Blot of FX-012 Under Non-Reduced (Lane 2) and Reduced (Lane 3) Conditions, Compared to a Molecular Weight Marker (Lane 1).

FIGS. 21A-21D show a flow diagram of (i) FX-012 cleavage by a SUMO protease (FIG. 21B) and (ii) its fusion to a thioester peptide (FIG. 21C). FIG. 21A is identical to the construct in FIG. 20B. FIG. 21B shows that, after FX-012 is cleaved by a SUMO protease, the resulting, cleaved polypeptide construct comprises three polypeptide chains, the first chain comprising FXLC, the second chain comprising Truncated FXHC (Factor X heavy chain without six amino acids (i.e., IVGGQE (SEQ ID NO: 85)) at the N-terminus) —Fc Region1—Proprotein Convertase Processing Site2, and the third chain comprising Fc Region2. The first chain and the second chain are bound by a disulfide bond, and the second and the third chains are bound by two disulfide bonds. FIG. 21C shows that after the cleaved polypeptide construct in FIG. 21B is ligated with a thioester peptide (D-Phe-Pip-Arg-PABC-IVGGQE-COSBn (SEQ ID NO: 90)). The resulting construct (TA-FX-012) comprises three chains, the first chain comprising FXLC, the second chain comprising Thrombin Cleavage Site (D-Phe-Pip-Arg-PABC)—FXHC—Fc Region1—Proprotein Convertase Processing Site, and the third chain comprising Fc Region 2. The first chain and the second chain are bound by a disulfide bond, and the second and the third chains are bound by two disulfide bonds. FIG. 21D shows reducing SDS-PAGE indicating the constructs and chains: lane 1 shows FX-012; lane 2 shows FX-012 with SUMO protease reaction; lane 3 shows FX-012 with SUMO protease reaction and conjugation with PABC peptide, lane 4 shows FX-012 with SUMO protease reaction and conjugation with a positive control peptide; and lane 5 shows marker. (—) indicates a peptide bond.

FIG. 22 shows FXa chromogenic assay after thrombin activation of TA-FX-012. X axis indicates time (min), and Y axis indicates Absorbance (A405) measurement for FXa activity. (x) shows FXa activity of a mixture of thrombin and hirudin. (◯) indicates FXa activity of TA- FX-012 without thrombin and hirudin. (Δ) indicates FXa activity of a mixture of TA-FXa-012, thrombin, and hirudin.

### DETAILED DESCRIPTION

The present disclosure provides procoagulant compounds comprising a protease-cleavable substrate (e.g., a synthetic thrombin substrate) and a self-immolative spacer (e.g., PABC) linked to a procoagulant polypeptide, e.g., a clotting factor or a procoagulant peptide. The protease-cleavable substrate can incorporate, e.g., the best known thrombin substrate, D-PhePipArg. Upon cleavage of the protease-cleavable substrate by a protease such as thrombin, the self-immolative spacer allows the release of the polypeptide via spontaneous fragmentation.

### I. Definitions

It must be noted that, as used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments of the disclosure, which can be had by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The term “sequence” as used to refer to a protein sequence, a peptide sequence, a polypeptide sequence, or an amino acid sequence means a linear representation of the amino acid constituents in the polypeptide in an amino-terminal to carboxyl-terminal direction in which residues that neighbor each other in the representation are contiguous in the primary structure of the polypeptide.

By a “protein” or “polypeptide” is meant any sequence of two or more amino acids linearly linked by amide bonds (peptide bonds) regardless of length, post-translation modification, or function. As used herein, the term “polypeptide” is intended to encompass a singular “polypeptide” as well as plural “polypeptides.” “Polypeptide,” “peptide,” and “protein” are used interchangeably herein. Thus, peptides, dipeptides, tripeptides, or oligopeptides are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. A polypeptide can be generated in any manner, including by chemical synthesis. Also included as polypeptides of the present invention are fragments, derivatives, analogs, or variants of the foregoing polypeptides, and any combination thereof

The term “fragment” when referring to polypeptides and proteins of the present invention include any polypeptides or proteins which retain at least some of the properties of the reference polypeptide or protein. E.g., in the case of procoagulant polypeptides such as clotting factors and procoagulant peptides, a term fragment would refer to any polypeptides or proteins which retain at least some of the procoagulant activity of the reference polypeptide or protein. Fragments of polypeptides include proteolytic fragments, as well as deletion fragments.

The term “variant” as used herein refers to a polypeptide sequence that differs from that of a parent polypeptide sequence by virtue of at least one amino acid modification. Variants can occur naturally or be non-naturally occurring. Non-naturally occurring variants can be produced using art-known mutagenesis techniques. Variant polypeptides can comprise conservative or non-conservative amino acid substitutions, deletions, or additions.

“Derivatives” of polypeptides or proteins of the invention are polypeptides or proteins which have been altered so as to exhibit additional features not found on the native polypeptide or protein. Also included as “derivatives” are those peptides that contain one or more naturally occurring amino acid derivatives of the twenty standard amino acids. A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide can have one or more mutations relative to the starting polypeptide, e.g., one or more amino acid residues which have been substituted with another amino acid residue or which has one or more amino acid residue insertions or deletions. Preferably, the polypeptide comprises an amino acid sequence which is not naturally occurring. Such variants necessarily have less than 100% sequence identity or similarity with the starting antibody. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In one embodiment, there is one amino acid difference between a starting polypeptide sequence and the sequence derived therefrom. Identity or similarity with respect to this sequence is defined herein as the percentage of amino acid residues in the candidate sequence that are identical (i.e. same residue) with the starting amino acid residues, after aligning the sequences and introducing gaps, if necessary, to achieve the maximum percent sequence identity.

A polypeptide which is “isolated” is a polypeptide which is in a form not found in nature. Isolated polypeptides include those which have been purified to a degree that they are no longer in a form in which they are found in nature. In some embodiments, a polypeptide which is isolated is substantially pure.

A “recombinant” polypeptide or protein refers to a polypeptide or protein produced via recombinant DNA technology. Recombinantly produced polypeptides and proteins expressed in host cells are considered isolated for the purpose of the invention, as are native or recombinant polypeptides which have been separated, fractionated, or partially or substantially purified by any suitable technique. The polypeptides disclosed herein, e.g., clotting factors or procoagulant peptides, can be recombinantly produced using methods known in the art. Alternatively, proteins and peptides disclosed herein can be chemically synthesized.

A “conservative amino acid substitution” is one in which the amino acid residue is replaced with an amino acid residue having a similar side chain. Families of amino acid residues having similar side chains have been defined in the art, including basic side chains (e.g., Lys, Arg, His), acidic side chains (e.g., Asp, Glu), uncharged polar side chains (e.g., Gly, Asn, Gnl, Ser, Thr, Tyr, Cys), nonpolar side chains (e.g., Ala, Val, Leu, Ile, Pro, Phe, Met, Trp), beta-branched side chains (e.g., Thr, Val, Ile) and aromatic side chains (e.g., Tyr, Phe, Trp, His). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, He, Phe or Val), (iii) a cysteine or proline is substituted for, or by, any other residue, or (iv) a residue having a bulky hydrophobic or aromatic side chain (e.g., VaI, He, Phe or Trp) is substituted for, or by, one having a smaller side chain (e.g., Ala, Ser) or no side chain (e.g., Gly).

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at tcoffee.org, and alternatively available, e.g., from the EBI. It will also be appreciated that the final alignment used to calculate percent sequence identity can be curated either automatically or manually.

As used herein, the term “half-life” refers to a biological half-life of a particular polypeptide (e.g., clotting factor or procoagulant peptide) or procoagulant compound of the invention in vivo. Half-life can be represented by the time required for half the quantity administered to a subject to be cleared from the circulation and/or other tissues in the animal. When a clearance curve of a given polypeptide or procoagulant compound of the invention is constructed as a function of time, the curve is usually biphasic with a rapid a-phase and longer β-phase. The a-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, procoagulant compounds of the invention are monophasic, and thus do not have an alpha phase, but just the single beta phase. Therefore, in certain embodiments, the term half-life as used herein refers to the half-life of the procoagulant compound in the β-phase. The typical β phase half-life of a human antibody in humans is 21 days.

The terms “heterologous” and “heterologous moiety” mean that a polynucleotide, polypeptide, or any other moiety which is derived from a distinct entity from that of the entity to which it is being compared. For instance, a heterologous polypeptide can be synthetic, or derived from a different species, different cell type of an individual, or the same or different type of cell of distinct individuals. In one embodiment, a heterologous moiety can be a polypeptide fused to another polypeptide to produce a fusion polypeptide or protein. In another embodiment, a heterologous moiety can be a non-polypeptide such as PEG conjugated to a polypeptide or protein.

As used herein, the terms “linked,” “fused”, “fusion,” or “connected” refer to linkage via a peptide bond (e.g., genetic fusion), chemical conjugation, or other means. For example, one way in which molecules or moieties can be linked employs polypeptide linkers which link the molecules or moieties via peptide bonds. The terms “genetically fused,” “genetically linked” or “genetic fusion” are used interchangeably and refer to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs.

As used herein the term “moiety” refers to a component part or constituent of a procoagulant compound of the invention.

As used herein, the term “targeting moiety” refers to heterologous moiety which localizes or directs the procoagulant compound of the invention to a desired site or cell. In one embodiment, the procoagulant compound of the invention comprises a “targeting moiety” which enhances the activity of the procoagulant compound, e.g., by localizing it to a desired site. Such a moiety can be, e.g., an antibody or variant thereof (e.g., and scFv) or a peptide. In another embodiment, the procoagulant compound of the invention comprises a targeting moiety which can be a polypeptide, a receptor binding portion of a ligand, or a ligand binding portion of a receptor and binds to the desired target, e.g., on a cell or tissue. In some embodiments, the procoagulant compound of the invention comprises a targeting moiety which is genetically fused, chemically conjugated, or linked to the construct via a linker or other moiety. Exemplary targeting moieties are described in more detail below. In one embodiment a targeting moiety for use in a procoagulant compound of the invention comprises an antibody or antibody variant. The term “antibody variant” or “modified antibody” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules; single-chain antibodies; diabodies; triabodies; and antibodies with altered effector function and the like.

As used herein the term “scFv molecule” includes binding molecules which consist of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. ScFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

A “scFv linker” as used herein refers to a moiety interposed between the VL and VH domains of the scFv. scFv linkers preferably maintain the scFv molecule in a antigen binding conformation. In one embodiment, a scFv linker comprises or consists of an scFv linker peptide. In certain embodiments, a scFv linker peptide comprises or consists of a gly-ser polypeptide linker. In other embodiments, a scFv linker comprises a disulfide bond.

As used herein, the term “protease-cleavable substrate” refers to peptide sequence comprising a site recognized by a protease enzyme. Certain cleavage sites comprise an intracellular processing site. In one embodiment, a procoagulant compound of the invention comprises a protease-cleavable substrate cleaved by an enzyme that is activated during the dotting cascade, such that cleavage of such sites occurs at the site of clot formation. Exemplary protease-cleavable substrates include, e.g., those recognized by thrombin, Factor XIa or Factor Xa. Exemplary FXIa cleavage sites include, e.g., TQSFNDFTR (SEQ ID NO: 2) and SVSQTSKLTR (SEQ ID NO: 3). Exemplary thrombin cleavage sites include, e.g., DFLAEGGGVR (SEQ ID NO: 4), TTKIKPR (SEQ ID NO: 5), LVPRG (SEQ ID NO: 6) and ALRPR (SEQ ID NO: 7). Other enzymatic cleavage sites are known in the art. Protease-cleavable substrates can comprise natural or non-natural amino acids, e.g., D-amino acids.

The term “bleeding disease or disorder,” as used herein, means a genetically inherited or acquired condition characterized by a tendency to hemorrhage, either spontaneously or as a result of trauma, due to an impaired ability or inability to form a fibrin clot. Examples of such disorders include hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for vWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk.

The phrase “effective amount” as used herein refers to that amount of a procoagulant compound or pharmaceutical composition of the present invention, which is effective for producing a desired effect, at a reasonable benefit/risk ratio applicable to any medical treatment. For example, an “effective amount” is an amount effective to reduce or lessen at least one symptom of the disease or disorder being treated or to reduce or delay onset of one or more clinical markers or symptoms associated with the disease or disorder, or to modify or reverse the disease process.

As used herein, the term “pharmaceutically acceptable” means approved by a regulatory agency of U.S. or E.U. or other government or listed in the U.S. Pharmacopeia or other generally recognized pharmacopeia for use in humans. Hence, the term “pharmaceutically acceptable” refers to those properties and/or substances that are acceptable to a patient (e.g., a human patient) from a toxicological and/or safety point of view.

The term “administering,” as used herein, means to give a procoagulant compound of the present invention, or pharmaceutical composition containing a procoagulant compound of the present invention, to a subject (e.g., human subject) in need thereof via a pharmaceutically acceptable route of administration. In some embodiments, the route of administration is intravenous, e.g., intravenous injection or intravenous infusion. In other embodiments, the route of administration is selected from subcutaneous, intramuscular, oral, nasal, and pulmonary administration. The procoagulant compounds of the invention can be administered as part of a pharmaceutical composition comprising at least one pharmaceutically acceptable carrier.

The term “prophylactic treatment,” as used herein, means administering a procoagulant compound of the present invention to a subject over a course of time to increase the level of activity in a subject's plasma. Preferably, the increased level is sufficient to decrease the incidence of spontaneous bleeding or to prevent bleeding, e.g., in the event of an unforeseen injury. Preferably, during prophylactic treatment, the plasma protein level in the subject does not fall below the baseline level for that subject, or below the level that characterizes severe hemophilia.

The term “subject,” as used herein means a human or a non-human mammal. Non-human mammals include, e.g., mice, dogs, primates, monkeys, cats, horses, cows, pigs, and other domestic animals and small animals.

The term “therapeutic dose,” as used herein, means a dose that achieves a therapeutic goal, as described herein. The therapeutic doses that can be used in the methods of the invention are about 10-100 mg/kg, more specifically, 10-20, 20-30, 30-40, 40-50, 50-60, 60-70, 70-80, 80-90, or 90-100 mg/kg, and more specifically, 10, 15, 20, 25, 30, 35, 40, 45, 50, 55, 60, 65, 70, 75, 80, 85, 90, 95, or 100 mg/kg. Additional therapeutic doses that can be used in the methods of the invention are about 10 to about 150 mg/kg, more specifically, about 100-110, 110-120, 120-130, 130-140, 140-150 mg/kg, and more specifically, about 110, 115, 120, 125, 130, 135, 140, 145, or 150 mg/kg.

“About,” as used herein for a range, modifies both ends of the range. Thus, “about 10-20” means “about 10 to about 20.”

The term “selective” as used in connection with proteolytic cleavage means a greater rate of cleavage of a protease-cleavable substrate relative to cleavage of a peptide substrate which comprises a random sequence of amino acids. The term “selective” also indicates that the procoagulant compound comprising the protease-cleavable substrate is cleaved at the site where it is coupled to the amino group of the self-immolative spacer.

The term “D-amino acid” as used herein, refers to an amino acid having a D-configuration (e.g., D-Phe). A D-amino acid can be a naturally occurring amino acid or an unnatural amino acid.

The term “self-immolative spacer” as used herein refers to a bifunctional chemical moiety which is capable of covalently linking together two spaced moieties (e.g., a clotting factor or a procoagulant peptide and a protein-cleavable substrate) into a normally stable tripartate molecule. The self-immolative spacer will spontaneously separate from the second moiety (e.g., a clotting factor or a procoagulant peptide) if its bond to the first moiety (e.g., a protein-cleavable substrate) is cleaved.

### II. Procoagulant Compounds

The present disclosure provides procoagulant compounds comprising a clotting factor or a fragment, variant, or derivative thereof, or a procoagulant peptide (e.g., a synthetic procoagulant peptide) connected to a protease-cleavable substrate (e.g., a thrombin substrate) via a self-immolative spacer (e.g., PABC). The PABC self-immolative spacer allows the release of any peptides and proteins containing at least one amine, phenol, carboxylic acid, or thiol functionality upon cleavage of the protease-cleavable substrate by endogenous or exogenous proteases and 1,6-spontaneous fragmentation. Cleavage kinetics is independent of the identity of released amine, phenol, carboxylic acid, or thiol molecules. Moreover, PABC can enhance the cleavage rate due to the presence of the p-aminobenzyl group.

In some embodiments, the disclosure provides a procoagulant compound represented by the following general formula:

(Het2)-(Pep2)-(Het1)-(L)-Zy-Bx-Pep1 (Formula I)

wherein:

Het1 is a first heterologous molecule, which is either absent or present;

Het2 is a second heterologous molecule, which is either absent or present;

L is a linker, which is either absent or present;

Zy is a protease-cleavable substrate;

Bx is a self-immolative spacer;

Pep1 is a first polypeptide; and,

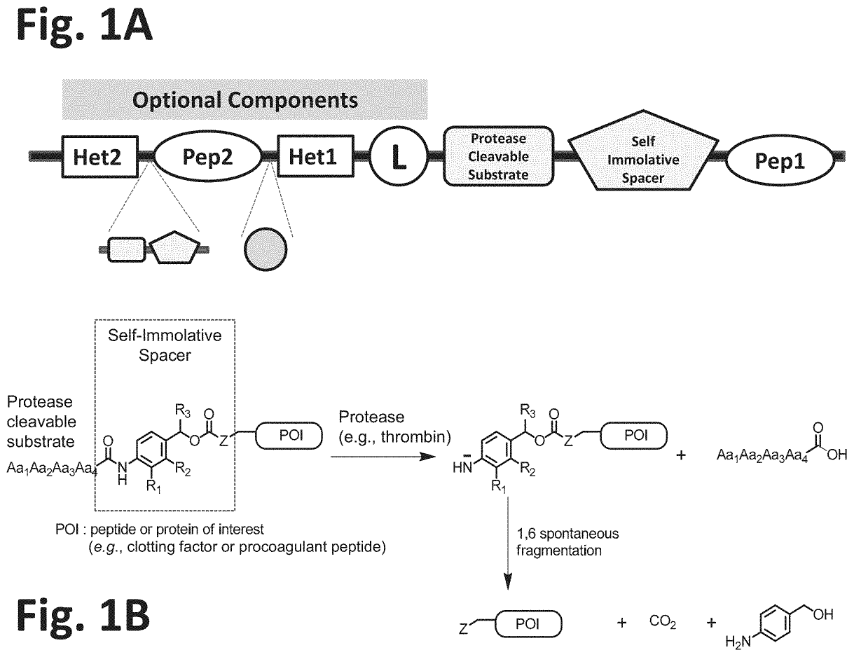

Pep2 is a second polypeptide, which is either absent or present;

wherein, Pep1 or Pep2 is a clotting factor or a fragment thereof, or a procoagulant peptide (for example, a synthetic procoagulant peptide) (see FIG. 1A).

In various embodiments, the present disclosure provides, inter alia, procoagulant compounds which are selectively activatable at the site of injury; procoagulant compounds that are selectively activatable by clotting cascade proteases; methods of treatment of bleeding disorders comprising the administration of the procoagulant compounds of the disclosure; methods for the production of the procoagulant compounds of the disclosure; and pharmaceutical compositions comprising the procoagulant compounds of the disclosure.

In some embodiments, the procoagulant compounds disclosed herein are stable and pharmacologically inactive in the absence of the protease targeting the protease-cleavable substrate. However, upon action of the protease, or any other suitable cleavage conditions, the protease-cleavable substrate is cleaved and the self-immolative spacer undergoes a spontaneous reaction, resulting in the release of an active procoagulant polypeptide (e.g., an active clotting factor or a procoagulant peptide). In some embodiments, the procoagulant compound of the invention is a zymogen.

In some aspects, a procoagulant protein of the invention comprises a formula selected from:
(a) Zy-Bx-Pep1;
(b) Het1-Zy-Bx-Pep1;
(c) Het1-L-Zy-Bx-Pep1;
(d) Pep2-Zy-Bx-Pep1;
(e) Pep2-L-Zy-Bx-Pep1;
(f) Pep2-Het1-L-Zy-Bx-Pep1;
(g) Pep2-Het1-Zy-Bx-Pep1;
(h) Het2-Het1-L-Zy-Bx-Pep1;
(i) Het2-Het1-Zy-Bx-Pep1;
(j) Het2-Pep2-Het1-L-Zy-Bx-Pep1;
(k) Het2-Pep2-L-Zy-Bx-Pep1;
(l) Het2-Pep2-Het1-Zy-Bx-Pep1; or,
(m) Het2-Pep2-Zy-Bx-Pep1,
   wherein:
Het1 is a first heterologous molecule;
Het2 is a second heterologous molecule;
L is a linker;
Zy is a protease-cleavable substrate;
Bx is a self-immolative spacer;
Pep1 is a first polypeptide; and,
Pep2 is a second polypeptide;
   wherein, Pep1 or Pep2 is a clotting factor or a fragment thereof, or a procoagulant peptide (for example, a synthetic procoagulant peptide).

In some embodiments, the formulas described herein can comprise additional sequences between the two moieties, e.g., linkers. For example, linkers can be situated between Het2 and Pep2, or between Pep2 and Het1. In some embodiments, additional Zy-Bx groups are present at the N-terminus of peptides (e.g., Pep1 or Pep2) and/or heterologous molecules (e.g., Het1 or Het2) to facilitate the clean release of such peptides and/or heterologous moieties. Accordingly, in some embodiments, a procoagulant protein of the invention comprises a formula selected from:

(n) Pep2-Zy-Bx-L-Zy-Bx-Pep1;

(o) Pep2-Zy-Bx-Het1-L- Zy-Bx-Pep1;

(p) Pep2-Zy-Bx-Het1-Zy-Bx-Pep1;

(q) Het2-Zy-Bx-Pep2-Zy-Bx-Hetl-L- Zy-Bx-Pep1;

(r) Het2-Zy-Bx-Pep2-Zy-Bx-L- Zy-Bx-Pep1;

(s) Het2-Zy-Bx-Pep2-Zy-Bx-Hetl-Zy-Bx-Pep1, or,

(t) Het2-Zy-Bx-Pep2-Zy-Bx-Pep1,

wherein:

Het1 is a first heterologous molecule;

Het2 is a second heterologous molecule;

L is a linker;

Zy is a protease-cleavable substrate;

Bx is a self-immolative spacer;

Pep1 is a first polypeptide; and,

Pep2 is a second polypeptide;

wherein, Pep1 or Pep2 is a clotting factor or a fragment thereof, or a procoagulant peptide (e.g., a synthetic procoagulant peptide).

The orientation of the procoagulant compounds formulas herein is listed from N-terminus (left) to C-terminus (right). For example, formula Pep2-Zy-Bx-Pep1 means formula NH₂-Pep2-Zy-Bx-Pep1-COOH. Formulas (a) to (t) shown above are included herein merely as non-limiting examples of procoagulant compounds of the present invention. For example, the formula Het2-Pep2-Hetl-L- Zy-Bx-Pep1 can further comprise sequences at the free end of Het2, between Het2 and Pep2, between Pep2 and Het1, between Het1 and Sp, between L and Zy, between Zy and Bx, between Bx and Pep1, or at the C-terminus of Pep1. In another embodiment, the hyphen (-) indicates a peptide bond or one or more amino acids.

In some embodiments, the procoagulant compound comprises a clotting factor or fragment thereof and a heterologous moiety. In some embodiments, the heterologous moiety comprises a half-life extending moiety selected, e.g., from the group consisting of an immunoglobulin constant region or portion thereof (e.g., an Fc region), a PAS sequence, HES, and albumin, fragment, or variant thereof, or an XTEN. In yet other embodiments, the procoagulant compound comprises a clotting factor or fragment thereof, a second clotting factor or fragment thereof, and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, or an XTEN.

In other embodiments, the procoagulant compound comprises a clotting factor or fragment thereof, a synthetic procoagulant polypeptide, and a heterologous moiety, wherein the procoagulant compound further comprises a second heterologous moiety selected from an immunoglobulin constant region or portion thereof (e.g., an Fc region), a PAS sequence, HES, and albumin, fragment, or variant thereof, XTEN, or any combinations thereof. In other embodiments, the procoagulant compound comprises two synthetic procoagulant peptides and a heterologous moiety, wherein the procoagulant compound further comprises a second heterologous moiety selected from an immunoglobulin constant region or portion thereof (e.g., an Fc region), a PAS sequence, HES, and albumin, fragment, or variant thereof, XTEN, or any combinations thereof. In yet another embodiment, the procoagulant compound comprises a clotting factor or fragment thereof, a clotting factor cofactor (e.g., Factor Va if the clotting factor in Factor X; or Tissue Factor if the clotting factor is Factor VII), and a heterologous moiety, wherein the procoagulant compound further comprises a second heterologous moiety selected from an immunoglobulin constant region or portion thereof (e.g., an Fc region), a PAS sequence, HES, and albumin, fragment, or variant thereof, XTEN, or any combinations thereof.

In specific aspects of the invention, a procoagulant compound of the invention comprises the formula wherein Hetl-L-Zy-Bx-Pep1, wherein Pep1 comprises a procoagulant peptide comprising the sequence rRAPGKLTCLASYCWLFWTGIA, Bx comprises a PABC self-immolative spacer, Zy comprises a thrombin-cleavable substrate comprising the sequence D-Phe-Pip-Arg, L comprises a linker comprising the sequence GGGG, and Het1 comprises a scaffold heterologous moiety comprising a cysteine amino acid.

In a specific aspect of the invention, a procoagulant compound of the invention comprises the formula wherein Zy-Bx-Pep1, wherein Pep1 comprises a FXa clotting factor, Bx comprises a PABC self-immolative spacer, and Zy comprises a thrombin-cleavable substrate comprising the sequence D-Phe-Pip-Arg.

In a specific aspect of the invention, a procoagulant compound of the invention comprises the formula wherein Zy-Bx-Pep1, wherein Pep1 comprises a FVIIa clotting factor, Bx comprises a PABC self-immolative spacer, and Zy comprises a thrombin-cleavable substrate comprising the sequence D-Phe-Pip-Arg.

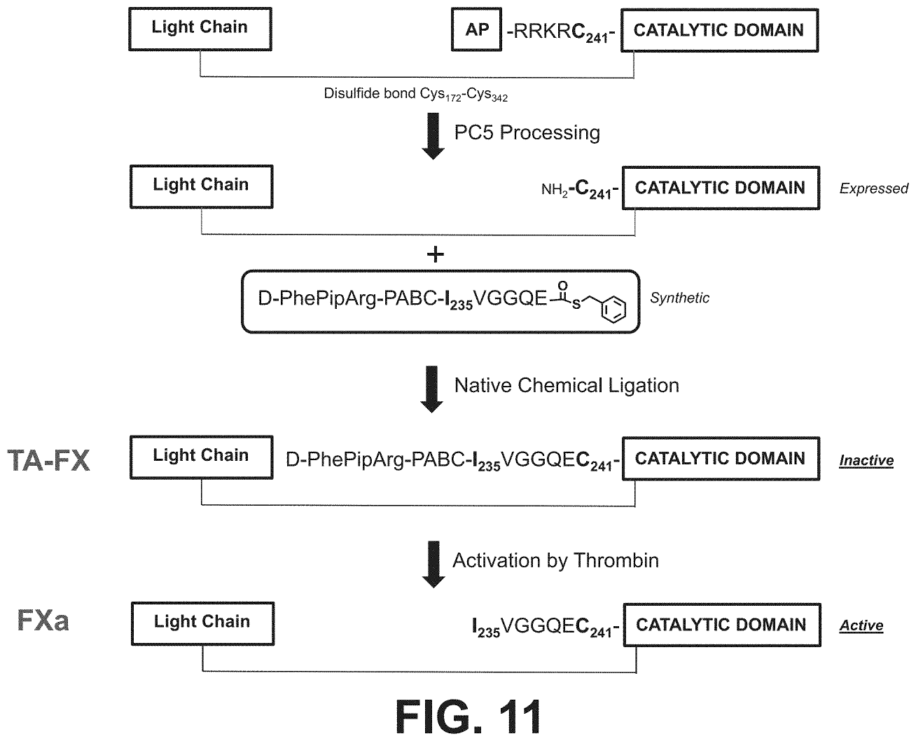

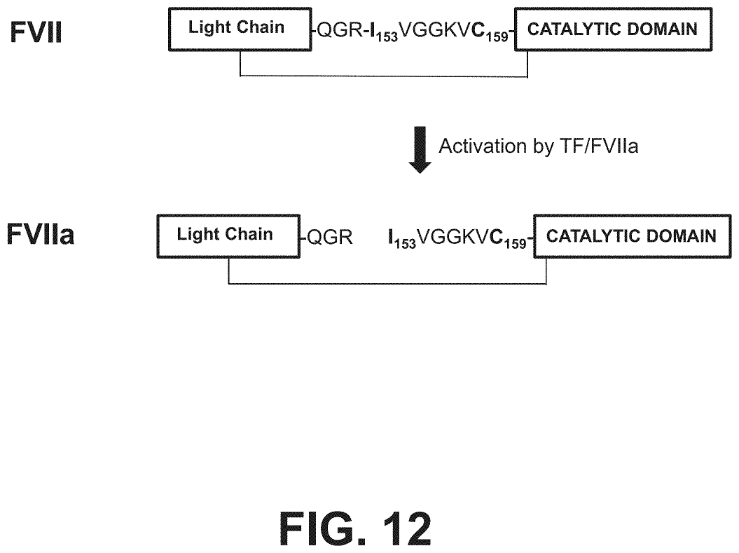

In another specific aspect of the invention, a procoagulant compound of the invention comprises the formula wherein Pep2-Zy-Bx-Pep1, wherein Pep1 comprises a FX clotting factor, Bx comprises a PABC self-immolative spacer, Zy comprises a thrombin-cleavable substrate comprising the sequence D-Phe-Pip-Arg, and Pep2 is an activation peptide, wherein cleavage of the thrombin-cleavable substrate causes the release of the activation peptide and the activation of the clotting factor (see FIG. 11). In some embodiments according to such formula, the clotting factor is FIXa and the activation peptide is FVIIIa. In other embodiments, the clotting factor is FVIIa and the activation peptide is Tissue Factor. In some embodiments, the activation peptide can be a procoagulant peptide.

In some specific aspects of the invention, a procoagulant compound of the invention comprises the formula Pep1-Het3-Het2-Bx-Het1, wherein Pep1 comprises a clotting factor, Het3 is a scaffold heterologous moiety, Het2 is a heterologous moiety, Bx is a self-immolative spacer, and Het1 is a second heterologous moiety. In some embodiments according to such formula, Pep1 comprises a FVIII clotting factor, Het3 is a cysteine, Het2 comprises an Fc heterologous moiety, Bx comprises a PABC self-immolative spacer, and Het) comprises an XTEN.

In some specific aspects of the invention, a procoagulant compound of the invention comprises the formula Pep1-Het2-Bx-Het1, wherein Pep1 comprises a clotting factor, Het2 is a scaffold heterologous moiety, Bx is a self-immolative spacer, and Het1 is a second heterologous moiety. In some embodiments according to such formula, Pep1 comprises a FVIII clotting factor, Het2 is a cysteine, Bx comprises a PABC self-immolative spacer, and Het) comprises an XTEN.

For a better understand of the procoagulant compounds of the disclosure their components will be discussed individually below:

### A. Polypeptides (e.g., Pep1, Pep2, . . . , Pepₙ)

### 1. Clotting Factors and Procoagulant Peptides

The procoagulant compounds the invention comprise at least one polypeptide moiety (Pep1 or Pep2) which is (i) a clotting factor, or (ii) a procoagulant peptide (e.g., a synthetic procoagulant peptide).

The term “clotting factor,” as used herein encompasses clotting factors (e.g., vWF, FV, FVa, FVII, FVIIa, FVIII, FVIIIa, FIX, FIXa, FX, FXa, FXI, FXIa, FXII, FXIIa, FXIII, or FXIIIa), fragments, variants, analogs, or derivatives thereof, naturally occurring, recombinantly produced, or synthetically produced which prevent or decrease the duration of a bleeding episode in a subject. In other words, it means molecules having procoagulant activity. In some embodiments, the procoagulant compound of the invention comprises a FVII or activated FVII (FVIIa) clotting factor. In other embodiments, the procoagulant compound of the invention comprises a FVIII or activated FVIII (FVIIIa) clotting factor. In some embodiments, the procoagulant compound of the invention comprises a FIX or activated FIX (FIXa) clotting factor. In other embodiments, the procoagulant compound of the invention comprises a FX or activated FX (FXa) clotting factor. In some embodiments, the procoagulant compound of the invention comprises vWF. The term “procoagulant peptide” as used herein refers to any peptide that has procoagulant activity. In particular, the term refers to peptides that initiate or accelerate the process of blood coagulation through the transformation of soluble circulating fibrinogen to an insoluble cross-linked fibrin network.

A “synthetic procoagulant peptide” as used herein refers to a procoagulant polypeptide that has been produced using solid phase peptide synthesis.

By “procoagulant activity” is meant the ability to promote thrombin generation and/or fibrin deposition in a suitable test system. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g. factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM analysis provides information on the whole kinetics of haemostasis:

clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

In some embodiments, the procoagulant compound comprises a single clotting factor. In some embodiments, the single clotting factor is Pep1. In other embodiments, the single clotting factor is Pep2. In other embodiments, the procoagulant compound comprises two clotting factors. In some embodiments, the two clotting factors are the same. In other embodiments, the two clotting factors are different. In some embodiments, one clotting factor is a fragment of a clotting factor (e.g., the heavy chain of a clotting factor such as FVIII) and the second clotting factor is a fragment of the same clotting factor (e.g., the light chain of a clotting factor such as FVIII). In some embodiments, the procoagulant compound comprises more than two clotting factors.

In some embodiments, a clotting factor's amino terminus is linked to a self-immolative spacer, which in turn is linked to a protease-cleavable substrate. In some embodiments, a procoagulant compound of the invention comprises two clotting factors (e.g., two different clotting factors or the heavy and light chains of a clotting factor) wherein only one of them has its amino terminus linked to a self-immolative spacer, which in turn is linked to a protease-cleavable substrate. In other embodiments, a procoagulant compound of the invention comprises two clotting factors (e.g., two different clotting factors or the heavy and light chains of a clotting factor) wherein both of them have its amino terminus linked to a self-immolative spacer, which in turn is linked to a protease-cleavable substrate.

In some embodiments, the procoagulant compound comprises a procoagulant peptide (e.g., a procoagulant synthetic peptide). In some embodiments, the procoagulant compound comprises two procoagulant peptides. In some embodiments, the two procoagulant peptides can be the same. In other embodiments, the two procoagulant peptides can be different. In some embodiments, at least one procoagulant peptide is a synthetic procoagulant peptide. In some embodiments, both procoagulant peptides are synthetic. In some embodiments, the procoagulant compound comprises more than two procoagulant peptides.

In some embodiments, the procoagulant compound comprises a clotting factor and a procoagulant peptide, e.g. a synthetic procoagulant peptide. In some embodiments, Pep1 is a clotting factor and Pep2 is a procoagulant peptide, e.g., a synthetic procoagulant peptide. In other embodiments, Pep1 is a procoagulant peptide, e.g., a synthetic procoagulant peptide, and Pep2 is a clotting factor.

In some embodiments, Pep1 and Pep2 are a clotting factor-clotting cofactor pair, e.g., FVIIa and tissue factor, FVIII and FIX, FVIII and vWF, FIXa and FVIIIa, etc.

Suitable clotting factors and procoagulant peptides to incorporate as Pep1 and/or Pep2 in procoagulant compounds of the invention are disclosed for example in WO2011/069164; WO 2012/006624; WO 2004/101740; WO/2007/112005; and WO 2012/006633; and in U.S. Provisional Patent Applications 61/491,762; 61/467,880; 61/442,029; 61/363,186; 61/599,305; 61/586,654; 61/586,099; 61/622,789; 61/586,443; 61/569,158; 61/541,561; 61/522,647; 61/506,015; 61/496,542; 61/496,541; 61/496,544; 61/496,543; or 3480000; all of which are herein incorporated by reference in their entireties.

### (i) Clotting Factors

In some embodiments, Pep1 and/or Pep2 are clotting factors, e.g., Factor VII, Factor VIII, Factor IX, and Factor X. Active forms of Factors VII, IX, and X are comprised of dimeric molecules in which the heavy and light chain are linked only by a disulfide bond. Methods for activating clotting factors are known in the art.

a. Factor VII

In some embodiments, a clotting factor is a mature form of Factor VII or a variant thereof. Factor VII (FVII, F7; also referred to as Factor 7, coagulation factor VII, serum factor VII, serum prothrombin conversion accelerator, SPCA, proconvertin and eptacog alpha) is a serine protease that is part of the coagulation cascade. FVII includes a Gla domain, two EGF domains (EGF-1 and EGF-2), and a serine protease domain (or peptidase S1 domain) that is highly conserved among all members of the peptidase S1 family of serine proteases, such as for example with chymotrypsin. FVII occurs as a single chain zymogen, an activated zymogen-like two-chain polypeptide and a fully activated two-chain form.

As used herein, a “zymogen-like” protein or polypeptide refers to a protein that has been activated by proteolytic cleavage, but still exhibits properties that are associated with a zymogen, such as, for example, low or no activity, or a conformation that resembles the conformation of the zymogen form of the protein. For example, when it is not bound to tissue factor, the two-chain activated form of FVII is a zymogen-like protein; it retains a conformation similar to the uncleaved FVII zymogen, and, thus, exhibits very low activity. Upon binding to tissue factor, the two-chain activated form of FVII undergoes conformational change and acquires its full activity as a coagulation factor.

Exemplary FVII variants include those with increased specific activity, e.g., mutations that increase the activity of FVII by increasing its enzymatic activity (Kcat or Km). Such variants have been described in the art and include, e.g., mutant forms of the molecule as described for example in Persson et al. 2001. PNAS 98:13583; Petrovan and Ruf 2001. J. Biol. Chem. 276:6616; Persson et al. 2001 J. Biol. Chem. 276:29195; Soejima et al. 2001. J. Biol. Chem. 276:17229; Soejima et al. 2002. J. Biol. Chem. 247:49027.

In one embodiment, a variant form of FVII includes the mutations Exemplary mutations include V158D-E296V-M298Q. In another embodiment, a variant form of FVII includes a replacement of amino acids 608-619 (LQQSRKVGDSPN, corresponding to the 170- loop) from the FVII mature sequence with amino acids EASYPGK from the 170-loop of trypsin. High specific activity variants of FIX are also known in the art. For example, Simioni et al. (2009 N.E. Journal of Medicine 361:1671) describe an R338L mutation. Chang et al. (1988 JBC 273:12089) and Pierri et al. (2009 Human Gene Therapy 20:479) describe an R338A mutation. Other mutations are known in the art and include those described, e.g., in Zogg and Brandstetter. 2009 Structure 17:1669; Sichler etl al. 2003. J. Biol. Chem. 278:4121; and Sturzebecher et al. 1997. FEBS Lett 412:295. The contents of these references are incorporated herein by reference.

Full activation, which occurs upon conformational change from a zymogen-like form, occurs upon binding to is co-factor tissue factor. Also, mutations can be introduced that result in the conformation change in the absence of tissue factor. Hence, reference to FVIIa includes both two-chain forms thereof: the zymogen-like form, and the fully activated two-chain form.

b. Factor VIII

In one embodiment, a clotting factor is a mature form of Factor VIII or a variant thereof. FVIII functions in the intrinsic pathway of blood coagulation as a cofactor to accelerate the activation of factor X by factor IXa, a reaction that occurs on a negatively charged phospholipid surface in the presence of calcium ions. FVIII is synthesized as a 2351 amino acid single-chain polypeptide having the domain structure A1-A2-B-A3-C1-C2. Wehar, G. A. et al., Nature 312:337-342 (1984) and Toole, J. J. et al., Nature 312:342-347 (1984).

The domain structure of FVIII is identical to that of the homologous coagulation factor, factor V (FV). Kane, W. H. et al., PNAS (USA) 83:6800-6804 (1986) and Jenny, R. J. et al., PNAS (USA) 84:4846-4850 (1987). The FVIII A-domains are 330 amino acids and have 40% amino acid identity with each other and to the A-domain of FV and the plasma copper-binding protein ceruloplasmin. Takahashi, N. et al., PNAS (USA) 81:390-394 (1984). Each C-domain is 150 amino acids and exhibits 40% identity to the C-domains of FV, and to proteins that bind glycoconjugates and negatively charged phospholipids. Stubbs, J. D. et al., PNAS (USA) 87:8417-8421 (1990). The FVIII B-domain is encoded by a single exon and exhibits little homology to any known protein including FV B-domain. Gitschier, J. et al., Nature 312:326-330 (1984) and Cripe, L. D. et al., Biochemistry 31:3777-3785 (1992).

FVIII is secreted into plasma as a heterodimer of a heavy chain (domains Al-A2-B) and a light chain (domains A3-C1-C2) associated through a divalent metal ion linkage between the A1- and A3-domains. In plasma, FVIII is stabilized by binding to von Willebrand factor. More specifically, the FVIII light chain is bound by noncovalent interactions to a primary binding site in the amino terminus of von Willebrand factor.

Upon proteolytic activation by thrombin, FVIII is activated to a heterotrimer of 2 heavy chain fragments (A1, a 50 kDa fragment, and A2, a 43 kDa fragment) and the light chain (A3-C1-C2, a 73 kDa chain). The active form of FVIII (FVIIIa) thus consists of an A1-subunit associated through the divalent metal ion linkage to a thrombin-cleaved A3-C1-C2 light chain and a free A2 subunit associated with the Al domain through an ion association. Eaton, D. et al., Biochemistry 25: 505 (1986); Lollar, P. et al., J. Biol. Chem. 266: 12481 (1991); and Fay, P. J. et al., J. Biol. Chem. 266: 8957 (1991). This FVIIIa heterotrimer is unstable and subject to rapid inactivation through dissociation of the A2 subunit under physiological conditions.

In one embodiment, a clotting factor comprises a B-domain deleted version of factor VIII. “B-domain” of Factor VIII, as used herein, is the same as the B-domain known in the art that is defined by internal amino acid sequence identity and sites of proteolytic cleavage, e.g., residues Ser741-Arg1648 of full-length human Factor VIII. The other human Factor VIII domains are defined by the following amino acid residues: A1, residues Ala1-Arg372; A2, residues Ser373-Arg740; A3, residues Ser1690-Asn2019; C1, residues Lys2020-Asn2172; C2, residues Ser2173-Tyr2332. The A3-C1-C2 sequence includes residues Ser1690-Tyr2332. The remaining sequence, residues Glu1649-Arg1689, is usually referred to as the a3 acidic region.

The locations of the boundaries for all of the domains, including the B-domains, for porcine, mouse and canine Factor VIII are also known in the art. In one embodiment, the B domain of Factor VIII is deleted (“B-domain-deleted factor VIII” or “BDD FVIII”). An example of a BDD FVIII is REFACTO® (recombinant BDD FVIII with S743/Q1638 fusion), which is known in the art.

A “B-domain-deleted Factor VIII” can have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. In some embodiments, a B-domain-deleted Factor VIII sequence comprises any one of the deletions disclosed at col. 4, line 4 to col. 5, line 28 and examples 1-5 of U.S. Pat. No. 6,316,226 (also in U.S. Pat. No. 6,346,513).

In another embodiment, a B-domain deleted Factor VIII is the S743/Q1638 B-domain deleted Factor VIII (SQ version Factor VIII) (e.g., Factor VIII having a deletion from amino acid 744 to amino acid 1637, e.g., Factor VIII having amino acids 1-743 and amino acids 1638-2332). In some embodiments, a B-domain-deleted Factor VIII of the present invention has a deletion disclosed at col. 2, lines 26-51 and examples 5-8 of U.S. Pat. No. 5,789,203 (also U.S. Pat. Nos. 6,060,447, 5,595,886, and 6,228,620).

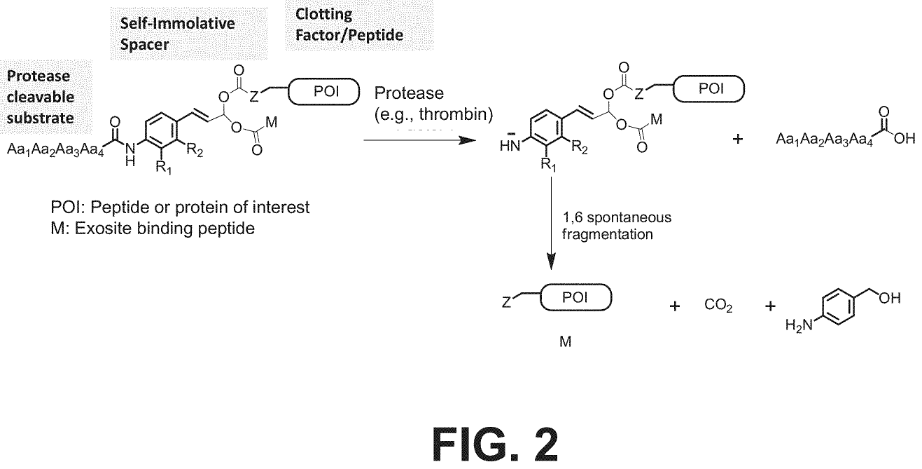

In some embodiments, a B-domain-deleted Factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563.

In some embodiments, a B-domain-deleted Factor VIII has a deletion of most of the B domain, but still contains amino-terminal sequences of the B domain that are essential for in vivo proteolytic processing of the primary translation product into two polypeptide chain, as disclosed in WO 91/09122, which is incorporated herein by reference in its entirety. In some embodiments, a B-domain-deleted Factor VIII is constructed with a deletion of amino acids 747-1638, i.e., virtually a complete deletion of the B domain. Hoeben R. C., et al. J. Biol. Chem. 265 (13): 7318-7323 (1990), incorporated herein by reference in its entirety.

A B-domain-deleted Factor VIII can also contain a deletion of amino acids 771-1666 or amino acids 868-1562 of Factor VIII. Meulien P., et al. Protein Eng. 2(4): 301-6 (1988), incorporated herein by reference in its entirety. Additional B domain deletions that are part of the invention include: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 though 1648 (Pasek (PCT application No.88/00831)), or 816 through 1598 or 741 through 1648 (Lagner (Behring Inst. Mitt. (1988) No 82:16-25, EP 295597)), each of which is incorporated herein by reference in its entirety. Each of the foregoing deletions can be made in any Factor VIII sequence.

c. Factor IX

In one embodiment, a clotting factor is a mature form of Factor IX or a variant thereof. Factor IX circulates as a 415 amino acid, single chain plasma zymogen (A. Vysotchin et al., J. Biol. Chem. 268, 8436 (1993)). The zymogen of FIX is activated by FXIa or by the tissue factor/FVIIa complex. Specific cleavages between arginine-alanine 145-146 and arginine-valine 180-181 result in a light chain and a heavy chain linked by a single disulfide bond between cysteine 132 and cysteine 289 (S. Bajaj et al., Biochemistry 22, 4047 (1983)).

The structural organization of FIX is similar to that of the vitamin K-dependent blood clotting proteins FVII, FX and protein C (B. Furie and B. Furie, supra). The approximately 45 amino acids of the amino terminus comprise the gamma-carboxyglutamic acid, or Gla, domain. This is followed by two epidermal growth factor homology domains (EGF), an activation peptide and the catalytic “heavy chain” which is a member of the serine protease family (A. Vysotchin et al., J. Biol. Chem. 268, 8436 (1993); S. Spitzer et al., Biochemical Journal 265, 219 (1990); H. Brandstetter et al., Proc. Natl. Acad Sci. USA 92, 9796 (1995)).

d. Factor X

In one embodiment, a clotting factor is a mature form of Factor X. Factor X is a vitamin-K dependent glycoprotein of a molecular weight of 58.5 kDa, which is secreted from liver cells into the plasma as a zymogen. Initially factor X is produced as a prepropeptide with a signal peptide consisting in total of 488 amino acids. The signal peptide is cleaved off by signal peptidase during export into the endoplasmatic reticulum, the propeptide sequence is cleaved off after gamma carboxylation took place at the first 11 glutamic acid residues at the N-terminus of the mature N-terminal chain. A further processing step occurs by cleavage between Arg182 and Ser183. This processing step also leads concomitantly to the deletion of the tripeptide Arg180-Lys181-Arg182. The resulting secreted factor X zymogen consists of an N-terminal light chain of 139 amino acids (M, 16,200) and a C-terminal heavy chain of 306 amino acids (M, 42,000) which are covalently linked via a disulfide bridge between Cys172 and Cys342. Further posttranslational processing steps include the β-hydroxylation of Asp103 as well as N- and O-type glycosylation.

It will be understood that in addition to wild type (WT) versions of these clotting factors or biologically active portions thereof, the present invention can also employ precursor truncated forms thereof that have activity, allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the mature form of the clotting factor and which retain the ability to promote clot formation. For example, modified FVII polypeptides and variants thereof which retain at least one activity of a FVII, such as TF binding, factor X binding, phospholipid binding, and/or coagulant activity of a FVII can be employed. By retaining activity, the activity can be altered, such as reduced or increased, as compared to a wild-type clotting factor so long as the level of activity retained is sufficient to yield a detectable effect.

Exemplary modified polypeptides include, but are not limited to, tissue-specific isoforms and allelic variants thereof, synthetic molecules prepared by translation of nucleic acids, proteins generated by chemical synthesis, such as syntheses that include ligation of shorter polypeptides, through recombinant methods, proteins isolated from human and non-human tissue and cells, chimeric polypeptides and modified forms thereof. The instant clotting factors can also consist of fragments or portions of WT molecules that are of sufficient length or include appropriate regions to retain at least one activity (upon activation if needed) of a full-length mature polypeptide. Exemplary clotting factor variants are known in the art.

As used herein, the term “Gla domain” refers to the conserved membrane binding motif which is present In vitamin K-dependent proteins, such as as prothrombin, coagulation factors VII, IX and X, proteins C, S, and Z. These proteins require vitamin K for the posttranslational synthesis of g-carboxyglutamic acid, an amino acid clustered in the N-terminal Gla domain of these proteins. All glutamic residues present in the domain are potential carboxylation sites and many of them are therefore modified by carboxylation. In the presence of calcium ions, the Gla domain interacts with phospholipid membranes that include phosphatidylserine. The Gla domain also plays a role in binding to the FVIIa cofactor, tissue factor (TF). Complexed with TF, the Gla domain of FVIIa is loaded with seven Ca2+ ions, projects three hydrophobic side chains in the direction of the cell membrane for interaction with phospholipids on the cell surface, and has significant contact with the C-terminal domain of TF.

The Gla domain of factor VII comprises the uncommon amino acid—carboxyglutamic acid (Gla), which plays a vital role in the binding of clotting factors to negatively charged phospholipid surfaces.

The GLA domain is responsible for the high-affinity binding of calcium ions. It starts at the N-terminal extremity of the mature form of proteins and ends with a conserved aromatic residue. A conserved Gla-x(3)-Gla-x-Cys motif is found in the middle of the domain which seems to be important for substrate recognition by the carboxylase.

Using stopped-flow fluorescence kinetic measurements in combination with surface plasmon resonance analysis, the Gla domain has been found to be important in the sequence of events whereby the protease domain of FVIIa initiates contact with sTF (Biochemical and Biophysical Research Communications. 2005. 337:1276). In addition, clearance of clotting factors can be significantly mediated through Gla interactions, e.g., on liver cells and clearance receptors, e.g., EPCR.

In one embodiment, targeted clotting factors are modified to lack a Gla domain. The Gla domain is responsible for mediating clearance of clotting factors via multiple pathways, such as binding to liver cells, clearance receptors such as EPCR, etc. Thus, eliminating the Gla domain has beneficial effects on half life of clotting factors. Though Gla domain is also generally required for activity by localizing clotting factors to sites of coagulation, the inclusion of a platelet targeting domain moiety targets the Gla deleted clotting factor to platelets. In one embodiment, a clotting factor comprises a targeting moiety and lacks a Gla domain. For example, in the case of Factor VII, the Gla domain is present at the amino terminus of the light chain and consists of amino acids 1-35. The Gla domains of exemplary clotting factors are indicated in the accompanying sequence listing. This domain can be removed using standard molecular biology techniques, replaced with a targeting domain, and the modified light chain incorporated into a construct of the invention. In one embodiment, a cleavage site can be introduced into constructs lacking a Gla domain to facilitate activation of the molecule. For example, in one embodiment, such a cleavage site can be introduced between the amino acids that are cleaved when the clotting factor is activated (e.g., between amino acids 152 and 153 in the case of Factor VII).

In one embodiment, a cleavage site can be introduced into constructs lacking a Gla domain to facilitate activation of the molecule. For example, in one embodiment, such a cleavage site can be introduced between the amino acids that are cleaved when the clotting factor is activated (e.g., between amino acids 152 and 153 in the case of Factor VII). Exemplary clotting factors lacking a Gla domain are shown in the accompanying figures.

Exemplary clotting factors are those of mammalian, e.g., human, origin.

### (ii) Procoagulant Peptides

Suitable procoagulant peptides to incorporate as Pep1 and/or Pep2 in procoagulant compounds of the invention are disclosed, for example, in U.S. Provisional Application Nos. 61/495,818; 61/600,237; and, 61/605,540 which are herein incorporated by reference in their entireties.

Exemplary synthetic procoagulant peptides include, for example:

(SEQ ID NO: 8)
KLTCLASYCWLF;
(SEQ ID NO: 9)
RRAPGKLTCLASYCWLFWTGIA;
(SEQ ID NO: 10)
RRAPGKLQCLASYCWLFWTGIA;
(SEQ ID NO: 11
PRIRTVGPGSRSASGKLTCLASYCWLFWTGIA;
(SEQ ID NO: 12)
SKQGRPISPDRRAAGKLTCLASYCWLFWTGIA;
(SEQ ID NO: 13)
PRIRTVGPGSRSASGKSTCLASYCWLFWTGIA;
(SEQ ID NO: 14)
SRIRTVSPGSRSASGKSTCLASYCWLFWTGIA;
or
(SEQ ID NO: 15)
PRSRTVGPGSRSASGKSTCLASYCWLFWTGIA.

### 2. Other Polypeptides

In some embodiments, the procoagulant compound comprises at least one polypeptide (e.g., Pep1 or Pep2) that is not a clotting factor or a synthetic procoagulant peptide. In some embodiments, the procoagulant compound comprises a Pep1 or Pep2 polypeptide which is clotting cascade cofactor, e.g., tissue factor, or a derivative, fragment, or variant thereof

In other embodiments, the procoagulant compound comprises a Pep1 or Pep2 polypeptide comprising a ligand binding moiety. In some embodiments, such ligand binding moiety is an antibody or an antigen binding fragment thereof.

### B. Self-immolative Spacer (Bx)

Procoagulant compounds according to the present disclosure comprise a self-immolative spacer. In some aspects, the self-immolative spacer comprises an aminobenzyl carbamate group, an aminobenzyl ether group, or an aminobenzyl carbonate group. In one aspect, the self-immolative spacer is p-amino benzyl carbamate (PABC).

P-amino benzyl carbamate (PABC) is the most efficient and most widespread connector linkage for self-immolative site-specific prodrug activation (see, e.g., Carl et al. J. Med. Chem. 24:479-480 (1981); WO 1981/001145; Rautio et la, Nature Reviews Drug Discovery 7:255-270 (2008); Simplicio et al., Molecules 13:519-547 (2008);). PABC allows the release of any amine drugs, peptides, and proteins upon cleavage by a protease and 1,6 spontaneous fragmentation (see FIG. 1).

In some embodiments, the self-immolative spacer connects a polypeptide of interest (e.g., a clotting factor or fragment thereof, or a synthetic procoagulant peptide) to a protease-cleavable substrate (e.g., a thrombin substrate). In specific aspects, the carbamate group of a PABC self-immolative spacer is connected to the N-terminus of a polypeptide of interest (e.g., a clotting factor or fragment thereof, or a synthetic procoagulant peptide), and the amino group of the PABC self-immolative spacer is connected to a protease-cleavable substrate (e.g., a thrombin substrate).

The aromatic ring of the aminobenzyl group can optionally be substituted with one or more (e.g., R₁ and/or R₂) substituents on the aromatic ring, which replace a hydrogen that is otherwise attached to one of the four non-substituted carbons that form the ring. As used herein, the symbol “Rₓ” (e.g., R₁, R₂, R₃, R₄) is a general abbreviation that represents a substituent group as described herein.

Substituent groups can improve the self-immolative ability of the p-aminobenzyl group (Hay et al., J. Chem Soc., Perkin Trans. 1:2759-2770 (1999); see also, Sykes et al. J. Chem. Soc., Perkin Trans. 1:1601-1608 (2000)).

The following formula shows the general topology of a p-amino benzyl immolative linker and the relative locations of an exemplary protease-cleavable substrate (Aa₂Aa₂Aa₃Aa₄) and a peptide or protein of interest (POI), which could be Pep1, Pep2, a polypeptide heterologous moiety, or linker comprising a peptide linker. The formula indicates possible locations of R substituent groups (R₁, R₂, R₃).

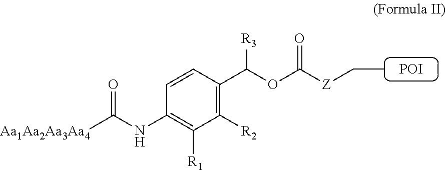

The substituents, which can be a single atom, e.g., a halogen, or a multi-atom group, e.g., methyl, are selected in order to impact the stability of the aminobenzyl or the decomposition product thereof. Electron withdrawal from the ring can be used to facilitate the spontaneous decomposition of the aminobenzyl group from the drug after cleavage of the bond between the amino group of the aminobenzyl group and the adjacent peptide linkage. Exemplary aromatic group R₁, R₂, or R₃ substituents include, for example, F, Cl, I, Br, OH, methyl, methoxy, NO₂, NH₂, NO³⁺, NHCOCH₃, N(CH₃)₂, NHCOCF₃, alkyl, haloalkyl, C₁-C8 alkylhalide, carboxylate, sulfate, sulfamate, sulfonate, etc. (see, e.g., U.S. Pat. Nos. 7,091,186 and 7,659,241). The p-aminobenzyl linker can comprise a heteroatom Z connected to the amino terminus of the peptide or protein of interest protein. The term heteroatom, as used herein, includes oxygen (O), nitrogen (N), sulfur (S), silicon (Si), boron (B) and phosphorus (P). In one embodiment, the heteroatoms in Z are O, S or N.

As illustrated below, in some aspects the self-immolative linker comprises an M group comprising an exosite binding peptide which binds to the exosite of its respective clotting factor.

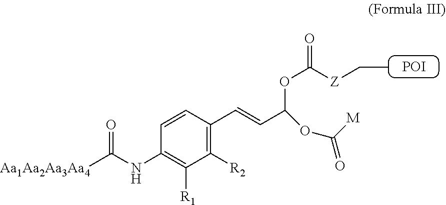

Many exosite binding motifs are known in the art. Insertion of an exosite binding motifs can increase the cleavage rate. Upon cleavage, both the exosite binding motif and the peptide or protein of interest (e.g., a clotting factor or fragment thereof, or a procoagulant peptide) are released (see FIG. 2).

In some embodiments, only one of the four non-substituted carbons in the p-aminobenzyl ring is substituted. In some other embodiments, two of the four non-substituted carbons in the p-aminobenzyl ring are substituted. In other embodiments, three of the four non-substituted carbons in the p-aminobenzyl ring are substituted. In some embodiments, the four non-substituted carbons in the p-aminobenzyl ring are substituted.

Self-immolative elimination can take place, e.g., via 1,4 elimination, 1,6 elimination (e.g., PABC), 1,8 elimination (e.g., p-amino-cinnamyl alcohol), β-elimination, cyclisation-elimination (e.g., 4-aminobutanol ester and ethylenediamines), cyclization/lactonization, cyclization/lactolization, etc. See, e.g., Singh et al. Curr. Med. Chem. 15:1802-1826 (2008); Greenwald et al. J. Med. Chem. 43:475-487 (2000).

In some aspects, the self-immolative spacer can comprise, e.g., an cinnamyl, naphthyl, or biphenyl groups (see, e.g., Blencowe et al. Polym. Chem. 2:773-790 (2011)).

In some aspects, the self-immolative spacer comprises a heterocyclic ring (see., e.g., U.S. Pat. Nos. 7,375,078; 7,754,681). Numerous homoaromatic (see, e.g., Carl et al. J. Med. Chem. 24:479 (1981); Senter et al. J. Org. Chem. 55:2975 (1990); Taylor et al. J. Org. Chem. 43:1197 (1978); Andrianomenjanahary et al. Bioorg. Med. Chem. Lett. 2:1903 (1992)), and coumarin (see, e.g., Weinstein et al. Chem. Commun. 46:553 (2010)), furan, thiophene, thiazole, oxazole, isoxazole, pyrrole, pyrazole (see, e.g., Hay et al. J. Med. Chem. 46:5533 (2003)), pyridine (see, e.g., Perry-Feigenbaum et al. Org. Biomol. Chem. 7:4825 (2009)), imidazone (see, e.g., Nailor et al. Bioorg. Med. Chem. Lett. Z:1267 (1999); Hay and Denny, Tetrahedron Lett. 38:8425 (1997)), and triazole (see, e.g., Bertrand and Gesson, J. Org. Chem. 72:3596 (2007)) based heteroaromatic groups that are self-immolative under both aqueous and physiological conditions are known in the art. See also, U.S. Pat Nos. 7,691,962; 7,091,186; U.S. Pat. Publ. Nos. US2006/0269480; US2010/0092496; US2010/0145036; US2003/0130189; US2005/0256030).

In some embodiments, a procoagulant compound of the invention comprises more than one self-immolative spacer in tandem, e.g., two or more PABC units. See, e.g., de Groot et al. J. Org. Chem. 66:8815-8830 (2001). In some embodiments, a procoagulant compound of the invention can comprise a self-immolative spacer (e.g., a p-aminobenzylalcohol or a hemithioaminal derivative of p-carboxybenzaldehyde or glyoxilic acid) linked to a fluorigenic probe (see, e.g., Meyer et al. Org. Biomol. Chem. 8:1777-1780 (2010)).

Where substituent groups in the self-immolative linkers are specified by their conventional chemical formulae, written from left to right, they equally encompass the chemically identical substituents, which would result from writing the structure from right to left. For example, “—CH₂O—” is intended to also recite “—OCH₂—”. Substituent groups in self-immolative, for example, R₁ and/or R₂ substituents in a p-aminobenzyl self-immolative linker as discuss above can include, e.g., alkyl, alkylene, alkenyl, alkynyl, alkoxy, alkylamino, alkylthio, heteroalkyl, cycloalkyl, heterocycloalkyl, aryl, arylalkyl, aryloxy, heteroaryl, etc. When a compound of the present disclosure includes more than one substituent, then each of the substituents is independently chosen.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tent-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one embodiment, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁₀, or C₂-C₈) and at least one heteroatom chosen , e.g., from N, O, S, Si, B and P (in one embodiment, N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—Si(CH₃)₃, —CH₂—CH═N—OCH₃, and —CH═CH—N(CH₃)—CH_{3.} Up to two heteroatoms can be consecutive, such as, for example, —CH₂—NH—OCH₃ and —CH₂—O—Si(CH₃)₃.

Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl,” “heterocyclic,” “heterocycle,” or “heterocyclyl,” by itself or in combination with other terms, represents a carbocyclic, non-aromatic ring (e.g., 3- to 8-membered ring and for example, 4-, 5-, 6- or 7-membered ring) containing at least one and up to 5 heteroatoms selected from, e.g., N, O, S, Si, B and P (for example, N, O and S), wherein the nitrogen, sulfur and phosphorus atoms are optionally oxidized, and the nitrogen atom(s) are optionally quatemized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule.

Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. In one embodiment, the aryl group is selected from phenyl, benzo[d][1,3]dioxolyl and naphthyl. The aryl group, in yet another embodiment, is phenyl.

The term “arylalkyl” or “aralkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” or “aralkyl” groups include benzyl, phenethyl, pyridylmethyl and the like.

By “aryloxy” is meant the group —O-aryl, where aryl is as defined herein. In one example, the aryl portion of the aryloxy group is phenyl or naphthyl. The aryl portion of the aryloxy group, in one embodiment, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si and B (for example, N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom.

In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahy drofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from acceptable aryl group substituents described below.

### C. Protease-Cleavable Substrate (Zy)

The procoagulant compounds of the invention comprise a protease-cleavable substrate (Zy) linked to a self-immolative spacer (Bx). In some embodiments, the procoagulant compound of the invention comprises a single protease-cleavable substrate. In other embodiments, particularly embodiments where more that one procoagulant polypeptide (e.g., a clotting factor o procoagulant peptide) or one or more heterologous moieties comprising polypeptide sequences are present, additional protease-cleavable substrate moieties (alone or in tandem with self-immolative spacers), can be linked to the N-terminus of the sequence of the procoagulant polypeptide or heterogous moiety. In some embodiments, protease-cleavable substrate moieties (alone or in tandem with self-immolative spacers), can be linked to the N-terminus of linkers (L) comprising peptide linkers.

Accordingly, procoagulant compounds of the invention can comprise a Zy-Bx-Pep1 module and further comprise, e.g., one or more of the following additional modules:

Zy-Het (wherein Het is Het1 or Het2),

Zy-Bx-Het (wherein Het is Het1 or Het2),

Zy-Pep (wherein Pep is Pep2 or an additional polypeptide if the procoagulant compound comprises more than two polypeptides)

Zy-Bx-Pep (wherein Pep is Pep2 or an additional polypeptide if the procoagulant compound comprises more than two polypeptides), wherein the additional module is covalently linked to the N-terminus or the C-terminus of the Zy-Bx-Pep1 module, with optionally one or more linkers or heterologous moieties interposed between the Zy-Bx-Pep1 and the additional module.

In some embodiments, when more that one protease-cleavable substrate is present, the substrates can be cleaved by the same or by different proteases. In embodiments where the protease-cleavable substrates are cleaved by the same protease, the protease-cleavable substrate can be the same or they can be different.

In some embodiments, the protease-cleavable substrate is a selective substrate for enzymatic cleavage by one or more proteases, e.g., blood-coagulation cascade proteases. Blood-coagulating cascade proteases include, but are not necessarily limited to, thrombin, FVIIa, FIXa, FXa, FXIa, and FXIIa.

The term “blood-coagulation cascade” used herein refers to the intrinsic, extrinsic, and common pathways. The intrinsic coagulation pathway leads to the formation of FIXa, that in conjunction with FVIIIa and FX, phospholipid and Ca2+ gives FXa. The extrinsic pathway gives FXa and FIXa after the combination of tissue factor and FVII. The common coagulation pathway interacts with the blood coagulation factors FV, FVIII, FIX and FX to cleave prothrombin to thrombin (FIIa), which is then able to cleave fibrinogen to fibrin.

In some embodiments, the protease-cleavable substrate comprise a cleavage site for a protease selected from neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases. enamelysin, tumor necrosis factor a-converting enzyme, insulysin, nardilysin, mitochondrial processing peptidase, magnolysin, dactylysin-like metal loproteases, neutrophil collagenase, matrix metallopeptidases, membrane-type matrix metalloproteinases, SP2 endopeptidase, prostate specific antigen (PSA), plasmin, urokinase, human fibroblast activation protein (FAPa), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose- binding protein-associated serine protease, coagulation factors, thrombin, protein c, u and t-type plasminogen activator, cathepsin G, hepsin, prostasin, hepatocyte growth factor- activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin D, pepsins, chymosyn, gastricsin, renin, yapsin and/or memapsins, Prostate-Specific antigen (PSA) , or any combinations thereof. See, e.g., Kohchi et al. Bioorganic & Medicinal Chemistry Letters 17:2241-2245 (2007); Brady et al. J. Med. Chem. 45:4706-4715 (2002).

In specific embodiments, the protease-cleavable substrate is selectively cleaved by thrombin at the site of injury. In some embodiments, the protease-cleavable substrate is selectively cleaved by thrombin in vitro, for example, when the procoagulant peptides of the invention are used for diagnosis or visualization. Non-limiting exemplary thrombin-cleavable substrates include, e.g., DFLAEGGGVR (SEQ ID NO: 4), TTKIKPR (SEQ ID NO: 5), or LVPRG (SEQ ID NO: 6), or a sequence comprising, consisting essentially of, or consisting of ALRPR (SEQ ID NO: 7) (e.g., ALRPRVVGGA (SEQ ID NO: 16)).

In specific embodiments, the thrombin-cleavable substrate comprises the sequence ALRPR (SEQ ID NO: 7), ALVPR (SEQ ID NO: 17), LVPR (SEQ ID NO: 18), D-Phe-Pro-Arg (SEQ ID NO: 19), D-Ala-Leu-Val-Pro-Arg (SEQ ID NO: 20), or D-Phe-Pip-Arg (Pip=pipecolic acid) (SEQ ID NO: 21) (see, e.g., Tung et al., ChemBioChem 3:207-2011 (2002); Jaffer et al. Arterioscler. Thromb. Vasc. Biol. 22:1929-1935 (2002); Rijkers et al. Thrombosis Research 79: 491-499 (1995)). Numerous synthetic thrombin-cleavable substrates are known in the art (see, e.g., Izquierdo & Burguillo, Int. J. Biochem. 21:579-592 (1989); WO1992/007869). The consensus cleavage site for thrombin has been identified as P3-hydrophobic, P2-Pro, Pip, P3-Arg, or isosteric and isolectronic with Arg; e.g., D-Phe-Pip-Lys (SEQ ID NO: 22), D-Phe-Pro-Lys (SEQ ID NO: 23), D-Phe-Pip-Orn (SEQ ID NO: 24). See also, Gallwitz et al. PLoS ONE 7(2): e31756. doi:10.1371/journal.pone.0031756 (2012); Tanihara et al. Peptides 19:421-425 (1998); Rijkers et al. Thrombosis 79:491-499 (1995).

In some embodiments, the protease-cleavable substrate comprises a FXIa cleavage site (e.g., KLTR↓AET (SEQ ID NO: 25)), a FXIa cleavage site (e.g., DFTR↓VVG (SEQ ID NO: 26)), a FXIIa cleavage site (e.g., TMTR↓IVGG (SEQ ID NO: 27)), a kallikrein cleavage site (e.g., SPFR↓STGG (SEQ ID NO: 28)), a FVIIa cleavage site (e.g., LQVR↓IVGG (SEQ ID NO: 29)), a FIXa cleavage site (e.g., PLGR↓IVGG (SEQ ID NO: 30)), a FXa cleavage site (e.g., IEGR↓TVGG (SEQ ID NO: 31)), a FIIa (thrombin) cleavage site (e.g., LTPR↓SLLV (SEQ ID NO: 32)), a Elastase-2 cleavage site (e.g., LGPV↓SGVP (SEQ ID NO: 33)), a Granzyme-B cleavage site (e.g, VAG↓NSLEE (SEQ ID NO: 34)), a MMP-12 cleavage site (e.g., GPAG↓LGGA (SEQ ID NO: 35)), a MMP-13 cleavage site (e.g., GPAG↓LRGA (SEQ ID NO: 36)), a MMP-17 cleavage site (e.g., APLG↓LRLR (SEQ ID NO: 37)), a MMP-20 cleavage site (e.g., PALP↓LVAQ (SEQ ID NO: 38)), a TEV cleavage site (e.g., ENLYFQ↓G (SEQ ID NO: 39)), a Enterokinase cleavage site (e.g., DDDK↓IVGG (SEQ ID NO: 40)), a Protease 3C (PRESCISSION™) cleavage site (e.g., LEVLFQ↓GP (SEQ ID NO: 41)), and a Sortase A cleavage site (e.g., LPKT↓GSES) (SEQ ID NO: 42). In certain embodiments, the FXIa cleavage sites include, but are not limited to, e.g., TQSFNDFTR (SEQ ID NO: 2) and SVSQTSKLTR (SEQ ID NO: 3).

### D. Linkers (L)

As described above, the procoagulant compounds of the invention can comprise one or more linkers. As used herein, the term “linker” (represented as L in the formulas disclosed herein) refers to a peptide or polypeptide sequence (e.g., a synthetic peptide or polypeptide sequence), or a non-peptide linker for which its main function is to connect two moieties (e.g., Het1, Het2, Pep1, Pep2, Bx) in a procoagulant compound of the invention. A linker can be present between any two moieties or non-linker elements of the procoagulant compounds of the invention. For example, one or more linkers can be present between a protease-cleavable substrate (e.g., a thrombin-cleavable substrate) and a heterologous moiety, or between a protease-cleavable substrate and a polypeptide (e.g., a procoagulant peptide, a clotting factor, or a non-procoagulant polypeptide), or between a first polypeptide and a second polypeptide, or between a first heterologous moiety and a second heterologous moiety. In some embodiments, two or more linkers can be linked in tandem.

When multiple linkers are present in a procoagulant compound of the invention, each of the linkers can be the same or different. Generally, linkers provide flexibility to the polypeptide molecule. Linkers are not typically cleaved; however in certain embodiments, such cleavage can be desirable. Accordingly, in some embodiments a linker can comprise one or more protease-cleavable sites, which can be located within the sequence of the linker or flanking the linker at either end of the linker sequence.

In one embodiment, the linker is a peptide linker. In some embodiments, the peptide linker can comprise at least two amino, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Examples of peptide linkers are well known in the art, for example peptide linkers according to the formula [(Gly)x-Ser_{y}]_{z} where x is from 1 to 4, y is 0 or 1, and z is from 1 to 50. In one embodiment, the peptide linker comprises the sequence Gn, where n can be an integer from 1 to 100. In a specific embodiment, the specific embodiment, the sequence of the peptide linker is GGGG. The peptide linker can comprise the sequence (GA)n. The peptide linker can comprise the sequence (GGS)n. In other embodiments, the peptide linker comprises the sequence (GGGS)ₙ (SEQ ID NO: 43). In still other embodiments, the peptide linker comprises the sequence (GGS)n(GGGGS)n (SEQ ID NO: 44). In these instances, n can be an integer from 1-100. In other instances, n can be an integer from 1-20, i.e., 1, 2, 3, 4, 5, 6, 7, 8, 9, 10, 11, 12, 13, 14, 15, 16, 17, 18, 19, or 20. Examples of linkers include, but are not limited to, GGG, SGGSGGS (SEQ ID NO: 45), GGSGGSGGSGGSGGG (SEQ ID NO:46), GGSGGSGGGGSGGGGS (SEQ ID NO:47), GGSGGSGGSGGSGGSGGS (SEQ ID NO:48), or GGGGSGGGGSGGGGS (SEQ ID NO:49). In other embodiments, the linker is a poly-G sequence (GGGG)ₙ (SEQ ID NO: 50), where n can be an integer from 1-100.

In one embodiment, the peptide linker is synthetic, i.e., non-naturally occurring. In one embodiment, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one embodiment the peptide linker can comprise non-naturally occurring polypeptides which are modified forms of naturally occurring polypeptides (e.g., comprising a mutation such as an addition, substitution or deletion). In another embodiment, the peptide linker can comprise non-naturally occurring amino acids. In another embodiment, the peptide linker can comprise naturally occurring amino acids occurring in a linear sequence that does not occur in nature. In still another embodiment, the peptide linker can comprise a naturally occurring polypeptide sequence.

In some embodiments, the linker comprises a non-peptide linker. In other embodiments, the linker consists of a non-peptide linker. In some embodiments, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyObutyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078).

Linkers can be introduced into polypeptide sequences using techniques known in the art (e.g., chemical conjugation, recombinant techniques, or peptide synthesis). Modifications can be confirmed by DNA sequence analysis. In some embodiments, the linkers can be introduced using recombinant techniques. In other embodiments, the linkers can be introduced using solid phase peptide synthesis. In certain embodiments, a procoagulant compound of the invention can contain simultaneously one or more linkers that have been introduced using recombinant techniques and one or more linkers that have been introduced using solid phase peptide synthesis or methods of chemical conjugation known in the art.

### E. Heterologous Moieties (e.g., Het1, Het2, . . . , Hetₙ)

In some embodiments, the procoagulant compound of the invention can comprise one heterologous moiety (indicated herein as “Het1” or “Het2”). In other embodiments, the procoagulant compound of the invention can comprise two heterologous moieties (“Het1” and “Het2”). In yet other embodiments, the procoagulant compound of the invention can comprise more than two heterologous moieties, e.g., three, four, five, or more than five heterologous moieties. In some embodiments, all the heterologous moieties are identical. In some embodiments, at least one heterologous moiety is different from the other heterologous moieties. In some embodiments, the procoagulant compound of the invention can comprise two, three or more than three heterologous moieties in tandem. In other embodiments, the procoagulant compound of the invention can comprise two, three, or more than heterologous moieties wherein at least an additional moiety (e.g., a procoagulant polypeptide, a linker, a protease-cleavable substrate, a self-immolative spacer, or combinations thereof) is interposed between two heterologous moieties.

A heterologous moiety can comprise a heterologous polypeptide moiety, or a heterologous non-polypeptide moiety, or both. In one specific embodiment, Het1 is a first heterologous moiety, e.g., a half-life extending molecule which is known in the art. In some embodiments, Het2 is a second heterologous moiety that can also be a half-life extending molecule which is known in the art. In some aspects, the heterologous moiety comprises a combination of a heterologous polypeptide and a non-polypeptide moiety.

In certain embodiments, the first heterologous moiety (e.g., a first Fc region) and the second heterologous moiety (e.g., a second Fc region) are associated with each other to form a dimer. In one embodiment, the second heterologous moiety is a second Fc region, wherein the second Fc region is linked to or associated with the first heterologous moiety, e.g., the first Fc region. For example, the second heterologous moiety (e.g., the second Fc region) can be linked to the first heterologous moiety (e.g., the first Fc region) by a linker or associated with the first heterologous moiety by a covalent or non-covalent bond.

In some embodiments, the Het1 and Het2 heterologous moieties are peptides and polypeptides with either unstructured or structured characteristics that are associated with the prolongation of in vivo half-life when incorporated in a procoagulant compound of the invention. Non-limiting examples include albumin, albumin fragments, Fc fragments of immunoglobulins, the 13 subunit of the C-terminal peptide (CTP) of the 13 subunit of human chorionic gonadotropin—a HAP sequence, XTEN, a transferrin or a fragment thereof, a PAS polypeptide, polyglycine linkers, polyserine linkers, albumin-binding moieties, or any fragments, derivatives, variants, or combinations of these polypeptides. In other related aspects a heterologous moiety can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety consisting of a cysteine amino acid that function as an attachment site for a non-polypeptide moiety such as polyethylene glycol (PEG), hydroxyethyl starch (HES), polysialic acid, XTEN, or any derivatives, variants, or combinations of these elements.

In some embodiments, the heterologous moiety is a polypeptide comprising, consisting essentially of, or consisting of at least about 10, 100, 200, 300, 400, 500, 600, 700, 800, 900, 1000, 1100, 1200, 1300, 1400, 1500, 1600, 1700, 1800, 1900, 2000, 2500, 3000, or 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous moiety improves one or more pharmacokinetic properties of the procoagulation compound without significantly affecting the biological activity or function of the Pep1 and/or Pep2 polypeptides (e.g., procoagulant activity of a clotting factor or a fragment thereof, or of procoagulant activity of a procoagulant synthetic peptide).

In certain embodiments, a heterologous moiety increases the in vivo and/or in vitro half-life of the procoagulant compound of the invention. In other embodiments, a heterologous moiety facilitates visualization or localization of the procoagulant compound of the invention or a fragment thereof (e.g., a fragment comprising a heterologous moiety after proteolytic cleavage of the protease-cleavable substrate Zy). Visualization and/or location of the procoagulant compound of the invention or a fragment thereof can be in vivo, in vitro, ex vivo, or combinations thereof

In other embodiments, a heterologous moiety increases stability of the procoagulant compound of the invention or a fragment thereof (e.g., a fragment comprising a heterologous moiety after proteolytic cleavage of the protease-cleavable substrate Zy). As used herein, the term “stability” refers to an art-recognized measure of the maintenance of one or more physical properties of the procoagulant compound in response to an environmental condition (e.g., an elevated or lowered temperature). In certain aspects, the physical property can be the maintenance of the covalent structure of the procoagulant compound (e.g., the absence of proteolytic cleavage, unwanted oxidation or deamidation). In other aspects, the physical property can also be the presence of the procoagulant compound in a properly folded state (e.g., the absence of soluble or insoluble aggregates or precipitates). In one aspect, the stability of the procoagulant compound is measured by assaying a biophysical property of the procoagulant compound, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

### 1. Half-Life Extending Heterologous Moieties

In certain aspects, a procoagulant compound of the invention comprises at least one half-life extending moiety, i.e., a heterologous moiety which increases the in vivo half-life of the procoagulant compound with respect to the in vivo half-life of the corresponding procoagulant compound lacking such heterologous moiety. In vivo half-life of a procoagulant compound can be determined by any method known to those of skill in the art, e.g., activity assays (chromogenic assay or one stage clotting aPTT assay), ELISA, etc.

In some embodiments, the presence of one or more half-life extending moieties results in the half-life of the procoagulant compound to be increased compared to the half life of the corresponding procoagulant compound lacking such one or more half-life extending moieties. The half-life of the procoagulant compound comprising a half-life extending moiety is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the in vivo half-life of the corresponding procoagulant compound lacking such half-life extending moiety.

In one embodiment, the half-life of the procoagulant compound comprising a half-life extending moiety is about 1.5-fold to about 20-fold, about 1.5 fold to about 15 fold, or about 1.5 fold to about 10 fold longer than the in vivo half-life of the corresponding procoagulant compound lacking such half-life extending moiety. In another embodiment, the half-life of procoagulant compound comprising a half-life extending moiety is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding procoagulant compound lacking such half-life extending moiety.

In other embodiments, the half-life of the procoagulant compound comprising a half-life extending moiety is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours.

In still other embodiments, the half-life of the procoagulant compound comprising a half-life extending moiety is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life per subject of the procoagulant compound comprising a half-life extending moiety is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

### (a) Low Complexity Polypeptides

In certain aspects, a procoagulant compound of the invention comprises at least one heterologous moiety comprising a polypeptide with low compositional and/or structural complexity (e.g., a disordered polypeptide with no secondary or tertiary structure in solution under physiologic conditions).

### (b) CTP

In certain aspects, a procoagulant compound of the invention comprises at least a heterologous moiety comprising one C-terminal peptide (CTP) of the 13 subunit of human chorionic gonadotropin, or fragment, variant, or derivative thereof. One or more CTP peptides inserted into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO: 51) or SSSSKAPPPSLPSPSRLPGPSDTPILPQ. (SEQ ID NO: 52). See, e.g., U.S. Patent Application Publication No. US 2009/0087411 A1, incorporated by reference.

### (c) Immunoglobulin Constant Region (Fe) or Portion Thereof

In certain aspects, a procoagulant compound of the invention comprises at least one Fc region. The term “Fc” or “Fc region” as used herein, means a functional neonatal Fc receptor (FcRn) binding partner comprising an Fc domain, variant, or fragment thereof which maintain the desirable properties of an Fc region in a chimeric protein, e.g., an increase in in vivo half-life. Myriad mutants, fragments, variants, and derivatives are described, e.g., in PCT Publication Nos. WO 2011/069164 A2, WO 2012/006623 A2, WO 2012/006635 A2, or WO 2012/006633 A2, all of which are incorporated herein by reference in their entireties. An Fc region is comprised of domains denoted CH (constant heavy) domains (CHL CH₂, etc.). Depending on the isotype, (i.e. IgG, IgM, IgA IgD, or IgE), the Fc region can be comprised of three or four CH domains. Some isotypes (e.g. IgG) Fc regions also contain a hinge region. See Janeway et al. 2001, Immunobiology, Garland Publishing, N.Y., N.Y.

An Fc region or a portion thereof for producing the procoagulant compound of the present invention can be obtained from a number of different sources. In some embodiments, an Fc region or a portion thereof is derived from a human immunoglobulin. It is understood, however, that the Fc region or a portion thereof can be derived from an immunoglobulin of another mammalian species, including for example, a rodent (e.g. a mouse, rat, rabbit, guinea pig) or non-human primate (e.g. chimpanzee, macaque) species. Moreover, the Fc region or a portion thereof can be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

Procoagulant compounds comprising an Fc region of an immunoglobulin bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

In certain embodiments, a procoagulant compound of the invention comprises one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH₂ domain and amino acid residues 385-387, 428, and 433-436 of the CH₃ domain. Thus, an Fc region in a procoagulant compound of the invention can comprise or consist of an FcRn binding portion. FcRn binding portions can be derived from heavy chains of any isotype, including IgGl, IgG2, IgG3 and IgG4. In one embodiment, an FcRn binding portion from an antibody of the human isotype IgG1 is used. In another embodiment, an FcRn binding portion from an antibody of the human isotype IgG4 is used.

In certain embodiments, an Fc region comprises at least one of: a hinge (e.g., upper, middle, and/or lower hinge region) domain (about amino acids 216-230 of an antibody Fc region according to EU numbering), a CH₂ domain (about amino acids 231-340 of an antibody Fc region according to EU numbering), a CH₃ domain (about amino acids 341-438 of an antibody Fc region according to EU numbering), a CH₄ domain, or a variant, portion, or fragment thereof. In other embodiments, an Fc region comprises a complete Fc domain (i.e., a hinge domain, a CH₂ domain, and a CH₃ domain). In some embodiments, an Fc region comprises, consists essentially of, or consists of a hinge domain (or a portion thereof) fused to a CH₃ domain (or a portion thereof), a hinge domain (or a portion thereof) fused to a CH₂ domain (or a portion thereof), a CH₂ domain (or a portion thereof) fused to a CH₃ domain (or a portion thereof), a CH₂ domain (or a portion thereof) fused to both a hinge domain (or a portion thereof) and a CH₃ domain (or a portion thereof). In still other embodiments, an Fc region lacks at least a portion of a CH₂ domain (e.g., all or part of a CH₂ domain). In a particular embodiment, an Fc region comprises or consists of amino acids corresponding to EU numbers 221 to 447.

An Fc in a procoagulant compound of the invention can include, for example, a change (e.g., a substitution) at one or more of the amino acid positions disclosed in Int'l. PCT Publs. WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; U.S. Pat. Publ. Nos. US 2007/0231329, US2007/0231329, US2007/0237765, US2007/0237766, US2007/0237767, US2007/0243188, US2007/0248603, US2007/0286859, US2008/0057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, each of which is incorporated by reference herein in its entirety. In one embodiment, the specific change (e.g., the specific substitution of one or more amino acids disclosed in the art) can be made at one or more of the disclosed amino acid positions. In another embodiment, a different change at one or more of the disclosed amino acid positions (e.g., the different substitution of one or more amino acid position disclosed in the art) can be made.

An Fc region used in the invention can also comprise an art recognized amino acid substitution which alters the glycosylation of the chimeric protein. For example, the Fc region of the procoagulant compound can comprise an Fc region having a mutation leading to reduced glycosylation (e.g., N- or O-linked glycosylation) or can comprise an altered glycoform of the wild-type Fc moiety (e.g., a low fucose or fucose-free glycan).

### (d) Albumin or Fragment, or Variant Thereof

In certain embodiments, the procoagulant compound of the invention comprises a heterologous moiety comprising albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, which are incorporated herein by reference in their entireties.

In one embodiment, the procoagulant compound of the invention comprises albumin, a fragment, or a variant thereof which is further linked to a heterologous moiety selected from an immunoglobulin constant region or portion thereof (e.g., an Fc region), a PAS sequence, HES, XTEN, PEG, or any combinations thereof

### (e) Albumin Binding Moiety

In certain embodiments, the heterologous moiety is an albumin binding moiety, which comprises an albumin binding peptide, a bacterial albumin binding domain, an albumin-binding antibody fragment, a fatty acid, or any combinations thereof

For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in US patent application 2003/0069395 or Dennis et al. (Dennis et al. (2002) J. Biol. Chem. 277, 35035-35043).

Domain 3 from streptococcal protein G, as disclosed by Kraulis et al., FEBS Lett.

378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO: 53). See, e.g., Dennis et al., J. Biol. Chem. 2002, 277: 35035-35043 (2002). Some examples of albumin-binding peptides are:

(SEQ ID NO: 54)
RLIEDICLPRWGCLWEDD,
(SEQ ID NO: 55)
QRLMEDICLPRWGCLWEDDF,
(SEQ ID NO: 56)
QGLIGDICLPRWGCLWGDSVK,
or
(SEQ ID NO: 57)
GEWWEDICLPRWGCLWEEED.

Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties. An example of such albumin binding moiety is 2-(3-maleimidopropanamido)-6-(4-(4-iodophenyObutanamido) hexanoate (“Albu” tag) as disclosed by Trussel et al., Bioconjugate Chem. 20:2286-2292 (2009).

Fatty acids, in particular long chain fatty acids (LCFA) and long chain fatty acid-like albumin-binding compounds can be used to extend the in vivo half-life of procoagulant compounds of the invention. An example of a LCFA-like albumin-binding compound is 16-(1-(3-(9-(((2,5-dioxopyrrolidin-1-yloxy)carbonyloxy)-methyi)-7-sulfo-9H-fluoren-2-ylamino)-3-oxopropyl)-2,5-dioxopyrrolidin-3-ylthio) hexadecanoic acid (see, e.g., WO 2010/140148).

### (f) PAS Sequence

In other embodiments, at least one heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the procoagulant compound. Yet, the skilled person is aware that an amino acid polymer also can form random coil conformation when residues other than alanine, serine, and proline are added as a minor constituent in the PAS sequence.

The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline can be selected from Arg, Asn, Asp, Cys, Gln, Glu, Gly, His, Ile, Leu, Lys, Met, Phe, Thr, Trp, Tyr, or Val.

Under physiological conditions, the PAS sequence stretch forms a random coil conformation and thereby can mediate an increased in vivo and/or in vitro stability to procoagulant compound. Since the random coil domain does not adopt a stable structure or function by itself, the biological activity mediated by the Pep1 and/or Pep2 polypeptides in the procoagulant compound is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable, which provides clear advantages over synthetic polymers such as PEG.

Non-limiting examples of the PAS sequences forming random coil conformation comprise an amino acid sequence selected from ASPAAPAPASPAAPAPSAPA (SEQ ID NO:58), AAPASPAPAAPSAPAPAAPS (SEQ ID NO:59), APSSPSPSAPSSPSPASPSS (SEQ ID NO:60), APSSPSPSAPSSPSPASPS (SEQ ID NO: 61), SSPSAPSPSSPASPSPSSPA (SEQ ID NO: 62), AASPAAPSAPPAAASPAAPSAPPA (SEQ ID NO: 63), ASAAAPAAASAAASAPSAAA (SEQ ID NO: 64) or any combinations thereof. Additional examples of PAS sequences are known from, e.g., US Pat. Publ. No. 2010/0292130 A1 and PCT Appl. Publ. No. WO 2008/155134 A1.

### (g) HAP Sequence

In certain embodiments, at least one heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

### (h) XTEN

In certain aspects, a procoagulant compound of the invention comprises at least one heterologous moiety comprising an XTEN polypeptide or fragment, variant, or derivative thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half life extension moiety. In addition, XTEN can provide desirable properties including but are not limited to enhanced pharmacokinetic parameters and solubility characteristics.

The incorporation of a heterologous moiety comprising an XTEN sequence into a procoagulant compound of the invention can confer to the procoagulant compound one or more of the following advantageous properties: conformational flexibility, enhanced aqueous solubility, high degree of protease resistance, low immunogenicity, low binding to mammalian receptors, or increased hydrodynamic (or Stokes) radii.

In certain aspects, an XTEN sequence can increase pharmacokinetic properties such as longer in vivo half-life or increased area under the curve (AUC), so that a procoagulant compound of the invention stays in vivo and has procoagulant activity for an increased period of time compared to a procoagulant compound with the same but without the XTEN heterogous moiety.

Examples of XTEN sequences that can be used as heterologous moieties in procoagulant compounds of the invention are disclosed, e.g., in U.S. Pat. Nos. 7,855,279 and 7,846,445, U.S. Patent Publication Nos. 2009/0092582 A1, 2010/0239554 A1, 2010/0323956 A1, 2011/0046060 A1, 2011/0046061 A1, 2011/0077199 A1, or 2011/0172146 A1, 2013/0017997 A1, or 2012/0263701 A1, or International Patent Publication Nos. WO 2010091122 A1, WO 2010144502 A2, WO 2010144508 A1, WO 2011028228 A1, WO 2011028229 A1, or WO 2011028344 A2, or International Application No. PCT/US2011/48517, filed Aug. 19, 2011, each of which is incorporated by reference herein in its entirety.

### (i) Transferrin or Fragment Thereof

In certain embodiments, at least one heterologous moiety is transferrin or a fragment thereof. Any transferrin can be used to make the procoagulant compounds of the invention. As an example, wild-type human Tf (TO is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and 595936 (ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric protein includes one or more domains of the transferrin sequence, e.g., N domain, C domain, N1 domain, N2 domain, C1 domain, C2 domain or any combinations thereof

### (j) Polymer, e.g., Polyethylene Glycol (PEG)

In other embodiments, at least one heterologous moiety is a soluble polymer known in the art, including, but not limited to, polyethylene glycol, ethylene glycol/propylene glycol copolymers, carboxymethylcellulose, dextran, or polyvinyl alcohol. In some embodiments, the procoagulant compound comprising a PEG heterologous moiety further comprises a heterologous moiety selected from an immunoglobulin constant region or portion thereof (e.g., an Fc region), a PAS sequence, HES, and albumin, fragment, or variant thereof, an XTEN, or any combinations thereof. In still other embodiments, the procoagulant compound comprises a clotting factor or fragment thereof and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, an XTEN, or any combinations thereof.

In yet other embodiments, the procoagulant compound comprises a clotting factor or fragment thereof, a second clotting factor or fragment thereof, and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, an XTEN, or any combinations thereof. In other embodiments, the procoagulant compound comprises a clotting factor or fragment thereof, a synthetic procoagulant polypeptide, and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, an XTEN, or any combinations thereof

In other embodiments, the procoagulant compound comprises two synthetic procoagulant peptides and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, an XTEN, or any combinations thereof. In yet another embodiment, the procoagulant compound comprises a clotting factor or fragment thereof, a clotting factor cofactor (e.g., Factor Va if the clotting factor in Factor X; or Tissue Factor if the clotting factor is Factor VII), and a PEG heterologous moiety, wherein the procoagulant compound further comprises a heterologous moiety selected from an immunoglobulin constant region or portion thereof (e.g., an Fc region), a PAS sequence, HES, and albumin, fragment, or variant thereof, an XTEN, or any combinations thereof

Also provided by the invention are procoagulant compounds of the invention comprising heterologous moieties which can provide additional advantages such as increased solubility, stability and circulating time of the polypeptide, or decreased immunogenicity (see U.S. Pat. No. 4,179,337). Such heterologous moieties for modification can be selected from water soluble polymers including, but not limited to, polyethylene glycol, ethylene glycol/propylene glycol copolymers, carboxymethylcellulose, dextran, polyvinyl alcohol, or any combinations thereof

The polymer can be of any molecular weight, and can be branched or unbranched. For polyethylene glycol, in one embodiment, the molecular weight is between about 1 kDa and about 100 kDa for ease in handling and manufacturing. Other sizes can be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol can have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

In some embodiments, the polyethylene glycol can have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

The number of polyethylene glycol moieties attached to each procoagulant compound of the invention (i.e., the degree of substitution) can also vary. For example, the PEGylated procoagulant compound can be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In some embodiments, the procoagulant compound can be PEGylated. A PEGylated procoagulant compound comprises at least one polyethylene glycol (PEG) molecule. In other embodiments, the polymer can be water-soluble. Non-limiting examples of the polymer can be poly(alkylene oxide), poly(vinyl pyrrolidone), poly(vinyl alcohol), polyoxazoline, or poly(acryloylmorpholine). Additional types of polymer-conjugation to clotting factors are disclosed in U.S. Pat. No. 7,199,223. See also, Singh et al. Curr. Med. Chem. 15:1802-1826 (2008).

### (k) Hydroxyethyl Starch (HES)

In certain embodiments, at least one heterologous moiety is a polymer, e.g., hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al.,Krankenhauspharmazie, 8(8), 271-278 (1987); and Weidler et al., Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et al., Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7.

In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD.

In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 1 1 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be a mixture of hydroxyethyl starches having different mean molecular weights and/or different degrees of substitution and/or different ratios of C2: C6 substitution. Therefore, mixtures of hydroxyethyl starches can be employed having different mean molecular weights and different degrees of substitution and different ratios of C2: C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2: C6 substitution, or having about the same mean molecular weight and about the same degree of substitution and about the same ratio of C2:C6 substitution.

(l) Polysialic Acids (PSA)

In certain embodiments, at least one heterologous moiety is a polymer, e.g., polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp 335-348. They can be produced in various degrees of polymerisation from n=about 80 or more sialic acid residues down to n=2 by limited acid hydrolysis or by digestion with neuraminidases, or by fractionation of the natural, bacterially derived forms of the polymer.

The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist—such as the alternating alpha-2,8 alpha-2,9 polysialic acid of E. coli strain K92 and group C polysaccharides of N. meningitidis. Sialic acid can also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals.

The alpha-2,8-linked polysialic acidof E. coli strain K1 is also known as ‘colominic acid’ and is used (in various lengths) to exemplify the present invention. Various methods of attaching or conjugating polysialic acids to a polypeptide have been described (for example, see U.S. Pat. No. 5,846,951; WO-A-0187922, and US 2007/0191597 A1, which are incorporated herein by reference in their entireties.

### (m) Clearance Receptors

In certain aspects, the in vivo half-life of a therapeutic polypeptide in a procoagulant compound of the invention can be extended where the procoagulant compound comprises at least one heterologous molecule comprising a clearance receptor, fragment, variant, or derivative thereof. In specific aspects wherein the therapeutic peptide is Factor VIII, Factor IX or Factor X, soluble forms of clearance receptors, such as the low density lipoprotein-related protein receptor LRP1, or fragments thereof, can block binding of Factor VIII, Factor IX or Factor X to clearance receptors and thereby extend its in vivo half-life.

LRP1 is a 600 kDa integral membrane protein that is implicated in the receptor-mediate clearance of a variety of proteins, such as Factor VIII. See, e.g., Lenting et al., Haemophilia 16:6-16 (2010). LRP1 also mediates clearance of Factor Xa (see, e.g., Narita et al., Blood 91:555-560 (1998)) and Factor IX (see, e.g., Strickland & Medved. J. Thromb. Haemostat. 4:1484-1486 (2006).

Other suitable FVIII clearance receptors are, e.g., LDLR (low-density lipoprotein receptor), VLDLR (very low-density lipoprotein receptor), and megalin (LRP-2), or fragments thereof. See, e.g.,Bovenschen et al., Blood 106:906-912 (2005); Bovenschen, Blood 116:5439-5440 (2010); Martinelli et al., Blood 116:5688-5697 (2010).

### 2. Visualization and Location

In certain embodiments, a heterologous moiety facilitates visualization or localization of the procoagulant compounds of the invention. Myriad peptides and other moieties for insertion or conjugation into a compound which facilitate visualization or localization are known in the art. Such moieties can be used to facilitate visualization or localization in vitro, in vivo, ex vivo or any combination thereof

Since thrombin plays a central role in the coagulation cascade, detection of imaging of its activity in vivo is highly desired. Accordingly, various heterologous moiety facilitates visualization or localization of the procoagulant compounds of the invention (e.g., fluorescent dyes) can be engineered into the procoagulant compounds of the invention. In some embodiments, fluorescent dyes can be engineered to be non-fluorescent until their amines are regenerated upon thrombin cleavage.

Non-limiting examples of peptides or polypeptides which enable visualization or localization include biotin acceptor peptides which can facilitate conjugation of avidin- and streptavidin-based reagents, lipoic acid acceptor peptides which can facilitate conjugation of thiol-reactive probes to bound lipoic acid or direct ligation of fluorescent lipoic acid analogs, fluorescent proteins, e.g., green fluorescent protein (GFP) and variants thereof (e.g., EGFP, YFP such as EYFP, mVenus, YPet or Citrine, or CFP such as Cerulean or ECFP) or red fluorescent protein (RFP), cysteine-containing peptides for ligation of biarsenical dyes such as 4′,5′-bis(1,3,2-dithioarsolan-2-yl)fluorescein (FlAsH), or for conjugating metastable technetium, peptides for conjugating europium clathrates for fluorescence resonance energy transfer (FRET)-based proximity assays, any variants, thereof, and any combination thereof

Procoagulant compounds of the present disclosure labeled by these techniques can be used, for example, for 3-D imaging of pathological thrombus formation and dissolution, tumor imaging in procoagulant malignancies, flow cytometric quantitation and characterization of procoagulant microparticles in blood and plasma, monitoring of thrombus formation by intravital microscopy.

### 3. Targeting Moieties, Anchors and Other Moieties

In some embodiments, procoagulant compounds of the invention can comprise a heterologous moiety that targets the compound to specific location, e.g., to platelets to enhance the efficacy of the compound by localizing the clotting factor or procoagulation peptide of the compound to the site of coagulation (a “targeting moiety”). In some embodiment, the targeting moiety binds to a target molecule expressed on platelets. Preferably the targeted molecules are not expressed on cells or tissues other than platelets, i.e., the targeting moieties specifically bind to platelets.

In one embodiment, receptors/conformations found on resting platelets are targeted. By doing so, sites for coagulation could be primed for enhanced efficacy. Targeting such molecule can also extend half life of the clotting factor and/or prevent clearance. Examples of such targets include GpIb of the Gplb/V/IX complex, and GpVI and nonactive form of GPIIb/IIIa. See, e.g., Schwarz et al. Circulation Research. 99:25-33 (2006); U.S. Pat. Publ. 20070218067; Peterson et al. Hemostasis, Thrombosis, and Vascular Biology 101:937 (2003); WO 2010115866; Lin et al. Journal of Thrombosis and Haemostasis 8:1773 (2010).

The procoagulant compound of the invention can comprise one or more than one targeting moiety. In some embodiments, two or more targeting moieties can be linked to each other (e.g., via a linker). When two or more targeting moieties are present in a procoagulant compound of the invention, the targeting moieties can be the same or different.

In one embodiment, a targeting moiety is fused to a procoagulant compound of the invention by a protease cleavable linker which can be cleaved to remove the targeting moiety at the site of a clot. In another embodiment, a targeting moiety is not attached via a cleavable linker and, therefore, is not cleaved at the site of a clot.

In one embodiment, the targeting moiety is located on the N- or C- terminus of the procoagulant compound. In one embodiment, a targeting moiety is not genetically fused directly to a procoagulant compound of the invention, but rather is chemically linked via a linker or a chemical bond to the construct (see, e.g., U.S. Pat. No. 7,381,408).

In one embodiment, a procoagulant compound of the invention comprises at least an antigen binding site (e.g., an antigen binding site of an antibody, antibody variant, or antibody fragment), a polypeptide, a receptor binding portion of ligand, or a ligand binding portion of a receptor which specifically binds to platelets, e.g., resting or activated platelets. Exemplary targeting moieties include scFv molecules or peptides which bind to molecules to be targeted.

In some embodiments, a procoagulant compound of the invention comprises an anchor or scaffolding molecule, e.g., a lipid, a carbohydrate, or a sulfhydryl group. For example, a sulfhydryl group in an N-terminal cysteine can be used to anchor a procoagulant compound of the invention to another molecule, cell, or other surface. For example, a lipid anchor can be used to anchor a procoagulant compound of the invention to a cell surface or a lipid bilayer (e.g., a liposome).

In some embodiments, a procoagulant compound of the invention can comprise a heterogous molecule comprising a non-peptidic active agent useful for the treatment of bleeding disorders. In some embodiments, the non-peptidic active agent is a procoagulant molecule. In some embodiments, the non-peptidic active agent is a small molecule drug.

### III. Pharmaceutical Compositions

The invention also provides pharmaceutical compositions containing at least one procoagulant compound of the present and a pharmaceutically acceptable carrier.

The term “pharmaceutically acceptable carrier” means all pharmaceutically acceptable ingredients known to those of skill in the art, which are typically considered non-active ingredients. The term “pharmaceutically acceptable carrier” includes, e.g., solvents, solid or liquid diluents, additives, vehicles, adjuvants, excipients, glidants, binders, granulating agents, dispersing agents, suspending agents, wetting agents, lubricating agents, disintegrants, solubilizers, stabilizers, preservatives, emulsifiers, fillers, preservatives (e.g., anti-oxidants), flavoring agents, sweetening agents, thickening agents, buffering agents, coloring agents and the like, as well as any mixtures thereof. Exemplary carriers (i.e., excipients) are described in, e.g., Handbook of Pharmaceutical Manufacturing Formulations, Volumes 1-6, Niazi, Sarfaraz K., Taylor & Francis Group 2005, which is incorporated herein by reference in its entirety.

Pharmaceutical compositions can additionally comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives.

Pharmaceutical compositions can be formulated for any appropriate manner of administration, including, for example, topical (e.g., transdermal or ocular), oral, buccal, nasal, vaginal, rectal or parenteral administration.

The term parenteral as used herein includes subcutaneous, intradermal, intravascular (e.g., intravenous), intramuscular, spinal, intracranial, intrathecal, intraocular, periocular, intraorbital, intrasynovial and intraperitoneal injection, as well as any similar injection or infusion technique. It is preferred that subcutaneous, intraperitoneal, buccal, intravenous and other parenteral formulations are sterile and endotoxin free. Procoagulant compounds of the present disclosure can be administered parenterally in a sterile medium.

The procoagulant compound, depending on the vehicle and concentration used, can either be suspended or dissolved in the vehicle. In one embodiment, adjuvants such as local anesthetics, preservatives and buffering agents can be dissolved in the vehicle. In one example, the procoagulant compounds of the present invention are administered to the subject using a non-intravenous route, e.g., by subcutaneous, nasal, buccal, oral or pulmonary delivery.

Forms suitable for oral use include, for example, tablets, troches, lozenges, aqueous or oily suspensions, dispersible powders or granules, emulsion, hard or soft capsules, or syrups or elixirs. Compositions provided herein can be formulated as a lyophilizate.

The pharmaceutical composition can be also for example a suspension, emulsion, sustained release formulation, cream, gel or powder. The pharmaceutical composition can be formulated as a suppository, with traditional binders and carriers such as triglycerides.

In one example, the pharmaceutical composition is a liquid formulation, e.g., a buffered, isotonic, aqueous solution. In one example, the pharmaceutical composition has a pH that is physiologic, or close to physiologic. In another example, the aqueous formulation has a physiologic or close to physiologic osmolarity and salinity. It can contain sodium chloride and/or sodium acetate.

Pharmaceutical compositions intended for oral use can be prepared according to any method known for the manufacture of pharmaceutical compositions. Such pharmaceutical compositions can contain one or more agents chosen from the group consisting of sweetening agents, flavoring agents, coloring agents and preservative agents in order to provide pharmaceutically elegant and palatable preparations. Tablets can contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period (i.e., tablets can be enterically coated). For example, a time delay material such as glyceryl monosterate or glyceryl distearate can be employed.

Pharmaceutical compositions for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. In another example, the active ingredient is formulated in capsules containing optionally coated microtablets or micropellets. Pharmaceutical compositions for oral use can also be presented as lozenges.

Aqueous suspensions contain the active ingredient(s) in admixture with excipients suitable for the manufacture of aqueous suspensions. Such excipients include suspending agents (e.g., sodium carboxymethylcellulose, methylcellulose, hydropropyl methylcellulose, sodium alginate, polyvinyl-pyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions can also comprise one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water can provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the present disclosure can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative, a flavoring agent or a coloring agent. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The procoagulant compounds of the present disclosure can also be administered in the form of suppositories, e.g., for rectal administration of the drug. These pharmaceutical compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Procoagulant compounds of the present disclosure can be formulated for local or topical administration, such as for topical application to the skin, wounds or mucous membranes, such as in the eye. Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices.

A composition can further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations can comprise microcapsules, such as hydroxymethylcellulose or gelatin-microcapsules, liposomes, albumin microspheres, microemulsions, nanoparticles or nanocapsules.

Pharmaceutical compositions suitable for topical administration to the eye also include eye drops wherein the active ingredients are dissolved or suspended in suitable carrier, especially an aqueous solvent for the active ingredients. The anti-inflammatory active ingredients may, for example, be present in such formulations in a concentration of 0.5 to 20%, such as 0.5 to 10%, for example about 1.5% w/w. For therapeutic purposes, the active compounds of the present disclosure are ordinarily combined with one or more adjuvants appropriate to the indicated route of administration. The compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose.

Formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof. The topical formulation can desirably include a compound, which enhances absorption or penetration of the active ingredient through the skin or other affected areas. Examples of such dermal penetration enhancers include dimethylsulfoxide and related analogs. The compounds of this present disclosure can also be administered by a transdermal device. In one embodiment, topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient.

If the active agent is absorbed through the skin, a controlled and predetermined flow of the active agent is administered to the recipient. In the case of microcapsules, the encapsulating agent can also function as the membrane. The transdermal patch can include the compound in a suitable solvent system with an adhesive system, such as an acrylic emulsion, and a polyester patch. The oily phase of the emulsions of this present disclosure can be constituted from known ingredients in a known manner. While the phase can comprise merely an emulsifier, it can comprise a mixture of at least one emulsifier with a fat or oil or with both a fat and an oil. In one embodiment, a hydrophilic emulsifier is included together with a lipophilic emulsifier, which acts as a stabilizer. The phase may, for example, include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations.

Emulsifiers and emulsion stabilizers suitable for use in the formulation of the present disclosure include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream may, for example, be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters can be used. These can be used alone or in combination depending on the properties required. Alternatively, high melting point lipids such as white soft paraffin and/or liquid paraffin or other mineral oils can be used.

A pharmaceutical composition can be formulated as inhaled formulations, including sprays, mists, or aerosols. For inhalation formulations, the compounds provided herein can be delivered via any inhalation methods known to those skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and can be filled into conventional aerosol containers that are closed by a suitable metering valve.

Formulations suitable for inhalation or insufflation include solutions and suspensions in pharmaceutically acceptable aqueous or organic solvents, or mixtures thereof, and powders. The liquid or solid compositions can contain suitable pharmaceutically acceptable excipients as describe above. The compositions can be administered by oral or nasal respiratory route for local or systemic effect. Compositions can be nebulized by use of inert gases or vaporized, and breathed directly from the nebulizing/vaporizing device or the nebulizing device can be attached to a facemask tent or intermittent positive pressure-breathing machine.

Inhalant compositions can comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Formulations or compositions suitable for nasal administration, wherein the carrier is a solid, include a coarse powder having a particle size, for example, in the range of 20 to 500 microns which is administered in the manner in which snuff is administered (i.e., by rapid inhalation through the nasal passage from a container of the powder held close up to the nose). Suitable powder compositions include, by way of illustration, powdered preparations of the active ingredient thoroughly intermixed with lactose or other inert powders acceptable for intrabronchial administration. The powder compositions can be administered via an aerosol dispenser or encased in a breakable capsule which can be inserted by the patient into a device that punctures the capsule and blows the powder out in a steady stream suitable for inhalation.

Pharmaceutical compositions can be formulated as sustained release formulations (i.e., a formulation such as a capsule that effects a slow release of modulator following administration). Such formulations can generally be prepared using well known technology and administered by, for example, oral, rectal or subcutaneous implantation, or by implantation at the desired target site. Carriers for use within such formulations are biocompatible, and can also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

In one example, the pharmaceutical formulations provided herein can include one or more additional active agent (i.e., other biologically active ingredient). In one example, the additional active agent is selected from known drugs approved for the treatment of a coagulation disorder, such as hemophilia A. For example, the pharmaceutical formulation can further include a blood coagulation factor.

Pharmaceutical compositions can be formulated with an agent to improve bioavailability, such an as organic solvent. For example, Cremophor EL.®. (Product No. 00647/1/63; BASF Aktiengesellschaft, Germany) is a polyethoxylated castor oil which is prepared by reacting 35 moles of ethylene oxide with each mole of castor oil. It can be used to stabilize emulsions of non-polar materials in aqueous systems. Alternatively, peptide, peptide derivative or dual peptide can be incorporated within or bound to a proteinaceous micro or nano-particle for improved bioavailability.

Suitable micro- and nano-particles are described in U.S. Pat. No. 5,439,686 (Desai et al; Vivorx Pharmaceuticals, Inc., CA) and U.S. Pat. No. 5,498,421 (Grinstaff et al; Vivorx Pharmaceuticals, Inc., CA). Suitably, the proteinaceous nano-particle comprises human serum albumin, particularly human serum albumin or a recombinant form thereof. WO 2007/077561 (Gabbai; Do-Coop Technologies Ltd., Israel) describe another suitable carrier comprising nanostructures and a liquid, referred to therein as NEOWATER™.

For veterinary use, a compound of the present disclosure is administered as a suitably acceptable formulation in accordance with normal veterinary practice and the veterinary surgeon will determine the dosing regimen and route of administration which will be most appropriate for a particular animal. For administration to non-human animals, the composition can be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

Dosage levels of the order of from about 0.005 mg to about 80 mg per kilogram of body weight per day are useful in the treatment of the diseases and conditions described herein (e.g., about 0.35 mg to about 5.6 g per human patient per day, based on an average adult person weight of 70 kg). The amount of active ingredient that can be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may, for example, be applied as a topical preparation of compounds of this present disclosure on the affected area one to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### IV. Methods of Making

The procoagulant compounds of the present disclosure can be produced by chemical synthesis, recombinant DNA technology, biochemical or enzymatic fragmentation of larger molecules, combinations of the foregoing or by any other method.

In one example, the method comprises forming the amino acid sequence of the compound, or a retro-, inverso- or retro-inverso variant thereof using solid-phase peptide synthesis. Exemplary methods of making procoagulant compounds of the invention are described herein in Example 1. Other methods to form synthetic peptides are known to those of skill in the art.

For example, the procoagulant compounds of the present disclosure can be synthesized using solid-phase peptide synthesis as described in “Fmoc Solid Phase Peptide Synthesis—A Practical Approach”, edited by W. C. Chan, P. D. White, Oxford University Press, New York 2000 and references therein. Temporary N-amino group protection is afforded, e.g., by a 9-fluorenylmethyloxycarbonyl (Fmoc) group. Repetitive cleavage of this highly base-labile protecting group is effected, e.g., using 20% piperidine in N,N-dimethylformamide. Side-chain functionalities can be protected as their butyl ethers (in the case of serine, threonine and tyrosine), butyl esters (in the case of glutamic acid and aspartic acid), butyloxycarbonyl derivative (in the case of lysine and histidine), trityl derivative (in the case of cysteine, asparagine and glutamine) and 4-methoxy-2,3,6-trimethylbenzenesulphonyl derivative (in the case of arginine).

The solid-phase support can be based on a polydimethyl-acrylamide polymer constituted from the three monomers dimethylacrylamide (backbone-monomer), bisacryloylethylene diamine (cross linker) and acryloylsarcosine methyl ester (functionalising agent), or can be based on polyethylene glycol (PEG), such as Rink Amide resin (e.g., NovaPEG Rink Amide). The peptide-to-resin cleavable linked agent can be the acid-labile 4-hydroxymethyl-phenoxyacetic acid derivative, or in case of C-terminal amides, the Rink-amide linker. All amino acid derivatives can be added as their preformed symmetrical anhydride derivatives with the exception of asparagine and glutamine, which are added using a reversed N,N-dicyclohexyl-carbodiimide/1-hydroxybenzotriazole mediated coupling procedure.

Alternatively, other peptide coupling reagents, such as O-benzotriazole-N,N,N′,N′-tetramethyl-uronium-hexafluoro-phosphate (HBTU) or 2-(6-chloro-1-H-benzotriazole-1-yl)-1,1,3,3-tetramethylaminium haxafluorophosphate (HCTU) can be used (e.g., in site). Coupling and deprotection reactions can be monitored using ninhydrin, trinitrobenzene sulphonic acid or isotin test procedures. Upon completion of synthesis, peptides are cleaved from the resin support with concomitant removal of side-chain protecting groups, e.g., by treatment with 95% trifluoroacetic acid containing about 5-50% scavenger. Scavengers commonly used are TIPS (triisopropylsilane), ethanedithiol, phenol, anisole water, and mixtures thereof. The exact choice depends on the constituent amino acids of the peptide being synthesised. For methionine containing peptides one can use, e.g., a mixture of TIPS (e.g., 2-5%) and ethanedithiol (e.g., 2-5%).

Trifluoroacetic acid can subsequently be removed by evaporation in vacuo, with subsequent trituration with diethyl ether affording the crude peptide. Any scavengers present can be removed by a simple extraction procedure which on lyophilisation of the aqueous phase affords the crude peptide free of scavengers.

Reagents for peptide synthesis are generally available, e.g., from Calbiochem-Novabiochem (UK), or EMD4Biosciences (U.S.).

Purification of the peptides can be effected by any one, or a combination of, techniques such as size exclusion chromatography, ion-exchange chromatography, affinity chromatography, differential solubility, and reverse-phase high performance liquid chromatography. Analysis of peptides can be carried out using thin layer chromatography, reverse-phase high performance liquid chromatography, mass spectroscopy (e.g., LC-MS), amino-acid analysis after acid hydrolysis and by fast atom bombardment (FAB) mass spectrometry.

SPOT-synthesis, which allows the positional addressable, chemical synthesis of peptides on continuous cellulose membranes can be also used (see, e.g., R. Frank, Tetrahedron (1992) 48, 9217).

When the procoagulant peptide is particularly large, e.g., larger than 50 amino acids, or larger than 100 amino acids, the procoagulant compounds of the present disclosure can be made semirecombinantly (see, e.g., U.S. Pat. No. 7,381,408; Dawson et al. Ann. Rev. Biochem. 69: 923-9600 (2000); Mei, B. et. al., Blood 116:270-279 (2010); and U.S. Pat. Appl. Publ. US2006/0115876, each of which is incorporated herein in its entirety). In one embodiment, a clotting factor or procoagulant polypeptide is produced recombinantly, and then attached to an intermediate compound comprising the cleavable substrate and self-immolative spacer via chemical ligation as described herein in Example 1. Chemical ligation can be performed using established organic chemistry techniques and commercially available reagents.

The procoagulant compounds of the invention can be assembled by conjugating the different moieties disclosed herein (e.g., polypeptides, heterologous moieties, linkers, protease-cleavable substrates, etc.) using orthogonal conjugation strategies know in the art. In some embodiments, such strategies include, e.g., alkyne, azide, N-terminal Cys, strained alkyne, ketone, aldehyde, tetrazine- trans-cyclooctene, and combinations thereof

In one aspect, the procoagulant compounds of the invention can be produced by using a cleavable polypeptide comprising a protease cleavable site, e.g., SUMO. Small Ubiquitin-like Modifier (or SUMO) is a member of the ubiquitin (Ub) and ubiquitin-like (Ubl) family. Post-translational attachment of SUMO to target proteins occurs through an enzymatic cascade analogous to the ubiquitin conjugation cascade (E1-E2-E3 enzymes), ultimately resulting in formation of an isopeptide bond between the Ub/Ubl C-terminal residue and substrate lysine residue.

SUMO Protease, a highly active cysteinyl protease also known as Ulp, is a recombinant fragment of Ulpl (Ubl-specific protease 1) from Saccharomyces cerevisiae. SUMO Protease cleaves in a highly specific manner, recognizing the tertiary structure of the ubiquitin-like (UBL) protein, SUMO, rather than an amino acid sequence. The protease can be used to cleave SUMO from recombinant fusion proteins. The sequence of the SUMO protein comprises:

(SEQ ID NO: 90)
SLQDSEVNQEAKPEVKPEVKPETHINLKVSDGSSEIFFKIKKTTPLRRLM
EAFAKRQGKEMDSLRFLYDGIRIQADQAPEDLDMEDNDITEAHREQIGG

In one embodiment, a cleavable polypeptide useful for the invention comprises a light chain of a clotting factor, a truncated heavy chain of the clotting factor, and a protease cleavable site, wherein the protease cleavable site is inserted between the truncated heavy chain of the clotting factor and the light chain of the clotting factor. In another embodiment, the cleavable polypeptide further comprises an intracellular processing site (also referred herein as proprotein convertase processing site) between the protease cleavable site and the light chain of the clotting factor. In other embodiments, a cleavable polypeptide comprises a light chain of a clotting factor, a protease cleavable site, a truncated heavy chain of the clotting factor, and a heterologous moiety, wherein the protease cleavable site is inserted between the light chain and the truncated heavy chain of the clotting factor, and the heterologous moiety is linked to the truncated heavy chain of the clotting factor by an optional linker. In still other embodiments, the protease cleavable site comprises SUMO, which can be cleaved by a SUMO protease.

In some embodiments, the heterologous moiety comprises a half-life extending moiety. Non-limiting examples of the half-life extending moiety are disclosed elsewhere herein. In certain embodiments, the clotting factor for the cleavable polypeptide comprises FVII or FX. In some embodiments, the truncated heavy chain of the clotting factor does not comprise one or more amino acids at the N-terminus of the truncated heavy chain compared to the wild-type heavy chain of the clotting factor. In certain embodiments, the one or more amino acids deleted from the truncated heavy chain of the clotting factor are two amino acids, three amino acids, four amino acids, five amino acids, six amino acids, seven amino acids, eight amino acids, nine amino acids, ten amino acids, eleven amino acids, twelve amino acids, thirteen amino acids, fourteen amino acids, or fifteen amino acids corresponding to the N-terminus of the heavy chain that are missing from the truncated heavy chain of the clotting factor. In one example, the clotting factor for the cleavable polypeptide is Factor VII and the one or more amino acids are IVGGKV (SEQ ID NO: 83). In another example, the clotting factor for the cleavable polypeptide is Factor X and the one or more amino acids are IVGGQE (SEQ ID NO: 85).

In certain embodiments, the invention is directed to a method of making a cleavable polypeptide comprising transfecting a host cell with a polynucleotide or a vector encoding a cleavable polypeptide under a condition sufficient to express the polypeptide. In some embodiments, the host cell further expresses a proprotein convertase, e.g., PACE or PCS, that can process any intracellular processing sites, e.g., 2X (RKR) (SEQ ID NO: 88) or RRRR (SEQ ID NO: 89).

In some aspects, a cleavable polypeptide comprising a light chain of a clotting factor, a protease cleavable site, and a truncated heavy cahin of the clotting factor is cleaved by a protease. After the cleavage, the resulting construct therefore can comprise at least two chains, the first chain comprising the light chain of the clotting factor and the second chain comprising the truncated heavy chain of the clotting factor. In some embodiments, the truncated heavy chain of the clotting factor lacks one or more amino acids at the end of the N-terminus compared to the wild-type clotting factor, thereby exposing Cysteine at the N-terminus for chemical ligation. In one embodiment, the amino acids missing from the truncated heavy chain are six amino acids, e.g., IVGGKV (SEQ ID NO: 83) for FVII or IVGGQE (SEQ ID NO 85) for FX. In another embodiment, the amino acids missing from the heavy cahin are 11 amino acids, e.g., IVGGKVCPKGE (SEQ ID NO: 84) for FVII or IVGGQECKDGE (SEQ ID NO: 86) for FX). In some embodiments, the protease cleavable site comprises SUMO.

In one aspect, the invention comprises a method of making a procoagulant compound comprising combining the cleavable polypeptide with a protease under a condition sufficient to cleave the protease cleavable site. In another embodiment, the method further comprises adding a thioester peptide to the cleaved polypeptide. The thioester peptide can then be fused to the N-terminus of the truncated heavy chain of the clotting factor, forming activatable clotting factor. In one example, the thioester peptide comprises a protease-cleavable substrate (Zy). In another embodiment, the thioester peptide comprises a protease-cleavable substrate and a self-immolative spacer (Bx), wherein the self-immolative spancer is inserted between the protease-cleavable substrate and the truncated heavy chain. In other embodiments, the thioester peptide comprises a protease-cleavable substrate, a self-immolative spacer, and one or more amino acids (W), wherein the self-immolative spacer is inserted between the protease-cleavable substrate and the one or more amino acids (W). In one aspect, the one or more amino acids comprise the N-terminus amino acid sequence missing from the truncated heavy chain of the clotting factor compared to the wild-type heavy chain. Therefore, in certain embodiments, the thioester peptide comprising a protease-cleavable substrate, a self-immolative moiety, and one or amino acids (e.g., six amino acids corresponding to wild-type heavy chain) forms protease-cleavable clotting factor, which comprises the protease-cleavable substrate, the self-immolative moiety, and full-length heavy chain. In some embodiments, the one or more amino acids comprise IVGGKV (SEQ ID NO: 83), wherein the clotting factor comprises Factor VII. In certain embodiments, the one or more amino acids comprise IVGGQE (SEQ ID NO: 85), wherein the clotting factor comprises Factor X. In other embodiments, the one or more amino acids comprise 11 amino acids, e.g., IVGGKVCPKGE (SEQ ID NO: 84) for FVII or IVGGQECKDGE (SEQ ID NO: 86) for FX). In yet other embodiments, the thioester peptide comprises a formula: Zy-Bx-W, wherein Zy is a protease-cleavable substrate; Bx is a self-immolative spacer; and W is one or more amino acids that are missing from the truncated heavy chain of the clotting factor. In a particular embodiment, the one or more amino acids and the truncated heavy chain of the clotting factor, when fused, result in the complete heavy chain of the clotting factor. In still other embodiments, the protease-cleavable substrate comprises a thrombin cleavage site, e.g., D-Phe-Pip-Arg. In yet other embodiments, the self-immolative spacer comprises PABC.

A variety of methods are available for recombinantly producing a clotting factor or procoagulant peptide for subsequent incorporation in a procoagulant compound of the invention. For recombinant production, a polynucleotide sequence encoding the clotting factor or procoagulant peptide is inserted into an appropriate expression vehicle, i. e. , a vector which contains the necessary elements for the transcription and translation of the inserted coding sequence, or in the case of an RNA viral vector, the necessary elements for replication and translation. The nucleic acid encoding the clotting factor or procoagulant peptide is inserted into the vector in proper reading frame. The expression vector is then transfected into a suitable target cell which will express the procoagulant polypeptide. Transfection techniques known in the art include, but are not limited to, calcium phosphate precipitation (Wigler et al. 1978, Cell 14:725) and electroporation (Neumann et al. 1982, EMBO, J. 1:841). A variety of host-expression vector systems can be utilized to express the procoagulant polypeptides described herein in eukaryotic cells. In one embodiment, the eukaryotic cell is an animal cell, including mammalian cells (e. g. 293 cells, PerC6, CHO, BHK, Cos, HeLa cells).

### V. Methods of Treatment

The present invention further provides methods for treating, ameliorating or preventing a bleeding disease or disorder in a subject (e.g., a human subject). An exemplary method comprises administering to the subject in need thereof a therapeutically effective amount of a procoagulant compound or a pharmaceutical composition/formulation of the present disclosure. In some embodiments, the procoagulant compounds or the pharmaceutical composition of the invention is administered to the subject orally.

The procoagulant compounds and pharmaceutical compositions of the invention can be used prophylactically. As used herein the term “prophylactic treatment” refers to the administration of a molecule prior to a bleeding episode. In one embodiment, the subject in need of a general hemostatic agent is undergoing, or is about to undergo, surgery. The procoagulant compound or pharmaceutical composition of the invention can be administered prior to or after surgery as a prophylactic. The procoagulant compound or pharmaceutical composition of the invention can be administered during or after surgery to control an acute bleeding episode. The surgery can include, but is not limited to, liver transplantation, liver resection, dental procedures, or stem cell transplantation.

The procoagulant compound or pharmaceutical composition of the invention can also used for on-demand treatment. The term “on-demand treatment” refers to the administration of a procoagulant compound or pharmaceutical composition of the invention in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

Treat, treatment, treating, as used herein refers to, e.g., the reduction in severity of a disease or disorder; the reduction in the duration of a disease course; the amelioration of one or more symptoms associated with a disease or disorder; the provision of beneficial effects to a subject with a disease or disorder, without necessarily curing the disease or disorder, or the prophylaxis of one or more symptoms associated with a disease or disorder.

In one example according to any of the above embodiments, the bleeding disease or disorder is caused by a blood coagulation disorder. A blood coagulation disorder can also be referred to as a coagulopathy. In a particular example, the blood coagulation disorder, which can be treated with a compound or a pharmaceutical composition of the current disclosure, is hemophilia or von Willebrand disease (vWD). In a particular example, the blood coagulation disorder, which can be treated with a compound or a pharmaceutical composition of the present disclosure is hemophilia A.

In another example, the type of bleeding associated with the bleeding disease or disorder is selected from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

In another example, the subject suffering from a bleeding disease or disorder is in need of treatment for surgery, including, e.g., surgical prophylaxis or peri-operative management. In one example, the surgery is selected from minor surgery and major surgery. Exemplary surgical procedures include tooth extraction, tonsillectomy, inguinal herniotomy, synovectomy, craniotomy, osteosynthesis, trauma surgery, intracranial surgery, intra-abdominal surgery, intrathoracic surgery, joint replacement surgery (e.g., total knee replacement, hip replacement, and the like), heart surgery, and caesarean section.

A coagulation disorder can be caused by a deficiency in at least one blood coagulation factor (e.g., FVIII). The current disclosure provides a method of treating a subject (e.g., a human subject) having a deficiency in at least one blood coagulation factor selected from von Willebrand Factor (vWF), FV, FVII, FVIIa, FVIII, FIX, FIXa, FX, FXI, and FXa (e.g., for both the prophylaxis and for the treatment of acute bleeds). An exemplary method comprises administering to the subject a therapeutically effective amount of a procoagulant compound or a pharmaceutical composition of the present disclosure.

In one example according to any of the above embodiments, the subject is a human subject (i.e., a human patient). In another example according to any of the above embodiments, the subject (e.g, human patient) is concomitantly treated with at least one additional active agent, e.g., a drug approved for the treatment of coagulation disorders. In one example, the additional active agent is administered to the subject at the same time that the procoagulant compound or pharmaceutical composition of the present disclosure is administered to the subject. For example, the at least one additional active agent is contained in a pharmaceutical composition that also contains the compound of the present disclosure. In another example, the additional active agent is administered to the subject at a different time but within the treatment period for the compound of the present disclosure. For example, the additional active agent is administered alternatingly with the procoagulant compound or pharmaceutical composition of the present disclosure.

For oral and parenteral administration to patients, including human patients, the daily dosage level of the procoagulant compound of the current disclosure will usually be from 2 to 2000 mg per adult (i.e. from about 0.03 to 30 mg/kg), administered in single or divided doses.

A unit dosage form (for example tablet or capsule) can contain from 2 mg to 2000 mg of procoagulant compound. The unit dosage form can be administered once, twice or more times per day as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

### VI. Other Methods

The invention further provides a method of increasing the efficacy of the cleavage of a protease-cleavage substrate (e.g., a thrombin-cleavable substrate) operably linked to a procoagulant polypeptide (e.g., a synthetic procoagulant peptide or clotting factor) comprising conjugating a self-immolative linker (e.g., PABC) to the procoagulant polypeptide, wherein the self-immolative linker is interposed between the protease-cleavage substrate and the procoagulant polypeptide.

In some embodiments, the efficacy of cleavage is increased by at least about 10%, by at least about 20%, by at least about 30%, by at least about 40%, by at least about 50%, by at least about 60%, by at least 70%, by at least about 80%, by at least 90% or by at least about 100% when compared to a reference procoagulant compound with the same sequence but without a self-immolative linker. In some embodiments, the efficacy of cleavage is increased by at least 100% when compared to a reference procoagulant compound with the same sequence but without a self-immolative linker.

In some embodiments, the efficacy of cleavage is at least about 2-fold, at least about 3-fold, at least about 4-fold, at least about 5-fold, at least about 6-fold, at least about 7-fold, at least about 8-fold, at least about 9-fold, or at least about 10-fold when compared to a reference procoagulant compound with the same sequence but without a self-immolative linker. In some embodiments, the efficacy of cleavage is at least about 20-fold, at least about 30-fold, at least about 40-fold, at least about 50-fold, at least about 60-fold, at least about 70-fold, at least about 80-fold, at least about 90-fold, or at least about 100-fold when compared to a reference procoagulant compound with the same sequence but without a self-immolative linker.

In some embodiments, wherein the procoagulant compound is cleaved by a protease specific for the protease-cleavable substrate moiety at least 10% faster when compared to a reference procoagulant compound with the same sequence but without a self-immolative linker. In some embodiments, cleavage is at least about 10%, at least about 20%, at least about 30%, at least about 40%, at least about 50%, at least about 60%, at least 70%, at least about 80%, at least 90% or at least about 100% faster when compared to a reference procoagulant compound with the same sequence but without a self-immolative linker. In some embodiments, the cleavage is faster by at least 100% when compared to a reference procoagulant compound with the same sequence but without a self-immolative linker. In some embodiments, the cleavage is at least about 2-fold, at least about 3-fold, at least about 4-fold, at least about 5-fold, at least about 6-fold, at least about 7-fold, at least about 8-fold, at least about 9-fold, or at least about 10-fold faster when compared to a reference procoagulant compound with the same sequence but without a self-immolative linker.

Also provided in the present disclosure is a method of activating a procoagulant peptide, comprising contacting a procoagulant compound of the invention a protease specific for the protease-cleavable substrate moiety in said procoagulant compound, wherein the activated procoagulant peptide is released upon proteolytic cleavage of the protease-cleavable substrate moiety.

The disclosure also provides a method of activating a clotting factor comprising contacting a procoagulant compound of the invention with a protease specific for the protease-cleavable substrate moiety in said procoagulant compound, wherein the activated clotting factor is released upon proteolytic cleavage of the protease-cleavable substrate moiety. In some embodiments, more than one clotting factor (e.g., an activated clotting factor), clotting factor fragment (e.g., a heavy chain or a light chain), procoagulant peptide (e.g., a synthetic procoagulant peptide) or combinations thereof can be released upon proteolytic cleavage of the procoagulant compound of the invention.

The disclosure also provides a method of releasing a procoagulant peptide from a heterologous moiety comprising contacting a procoagulant compound of the invention with a protease specific for the protease-cleavable substrate in said procoagulant compound, wherein the activated procoagulant polypeptide is released upon proteolytic cleavage of the protease-cleavable substrate.

The disclosure also provides a method of releasing a clotting factor from a heterologous moiety comprising contacting a procoagulant compound of the invention with a protease specific for the protease-cleavable substrate in said procoagulant compound, wherein the activated clotting factor is released upon proteolytic cleavage of the protease-cleavable substrate. In some embodiments, more than one clotting factor (e.g., an activated clotting factor), clotting factor fragment (e.g., a heavy chain or a light chain), procoagulant peptide (e.g., a synthetic procoagulant peptide) or combinations thereof can be released upon proteolytic cleavage of the procoagulant compound of the invention from one or more heterologous moieties (e.g., PEG). In some embodiments, the release of the one or more than one clotting factor (e.g., an activated clotting factor), clotting factor fragment (e.g., a heavy chain or a light chain), procoagulant peptide (e.g., a synthetic procoagulant peptide) or combinations thereof factor and the heterologous moiety takes place simultaneously. In some embodiments, the release of the release of the one or more than one clotting factor (e.g., an activated clotting factor), clotting factor fragment (e.g., a heavy chain or a light chain), procoagulant peptide (e.g., a synthetic procoagulant peptide) or combinations thereof factor and the release of the heterologous moiety takes place sequentially.

Also provided in the disclosure is a method of releasing at least one heterologous moiety from a procoagulant compound of the invention, prior or concurrently with the release of one or more clotting factors, clotting factor fragments (e.g., a heavy chain or a light chain), procoagulant peptides (e.g., a synthetic procoagulant peptide) or combinations thereof by treatment with one or more proteases, comprising contacting a procoagulant compound of the invention with one or more proteases specific for the one or more than one protease-cleavable substrate in said procoagulant compound. In some embodiments, the release of one, two, three or more than three heterologous moieties takes place prior to the proteolytic release of clotting factors, clotting factor fragments (e.g., a heavy chain or a light chain), procoagulant peptides (e.g., a synthetic procoagulant peptide) or combinations thereof. In other embodiments, the release of one, two, three or more than three heterologous moieties takes place concurrently with the proteolytic release of clotting factors, clotting factor fragments (e.g., a heavy chain or a light chain), procoagulant peptides (e.g., a synthetic procoagulant peptide) or combinations thereof. In some embodiments, the release of two, three or more than three heterologous moieties takes place simultaneously. In some embodiments, the release of two, three or more than three heterologous moieties takes place sequentially.

Having now described the present invention in detail, the same will be more clearly understood by reference to the following examples, which are included herewith for purposes of illustration only and are not intended to be limiting of the invention. All patents, patent application, patent application publications, and other publications referred to herein are expressly incorporated by reference in their entireties.

### EXAMPLES

### Materials and Methods

The materials and methods for peptide synthesis, purification, and characterization described below are used in the Examples below unless otherwise stated.

### 1. Solid Phase Peptide Synthesis

Synthetic procoagulant peptides of the present disclosure were synthesized by solid phase peptide synthesis using 9-fluorenylmethoxycarbonyl/tertiary-butyl (Fmoc/tBu) chemistry. Heating was accomplished using a microwave oven or other means. In most cases, the peptides were synthesized in 0.1 mmol scale using NovaPEG Rink Amide resin (Novabiochem) or NovaPEG TGT resin (Novabiochem) in a 35 mL reaction vessel. Standard methods for resin load, amino acid coupling, Fmoc deprotection and washing steps were performed on a CEM Liberty peptide synthesizer (CEM Corp.), whereas the trifluoroacetic acid (TFA) cleavage of the peptide was performed manually.

Briefly, 5 equivalent of Fmoc protected amino acids dissolved in N,N-dimethylformamide (DMF) were linked subsequently to the resin in the presence of 5 equivalents of 2(6-chloro-1H-benzotriazole-1-yl)-1,1,3,3-tetramethylaminium hexafluoro phosphate (HCTU) and 10 equivalents of diisopropylethylamine (DIPEA). The microwave method used for the coupling step was single coupling at 75° C. (20W for 300 seconds), except for cysteine and histidine, which were coupled at 50° C. (OW for 120 sec, 20W for 240 seconds). Arginine was double coupled at 75° C. (OW for 1500 sec, 20W for 300 seconds). The Fmoc deprotection was performed with 5% piperazine, 0.1M 1-hydroxybenzotriazole (HOBt) in DMF at 75° C. (45W for 30 seconds, 45W for 180 seconds). Most amino acids and coupling reagents were purchased from Novabiochem® EMD (EMD Millipore Chemicals).

Following the automated peptide synthesis, the peptides were cleaved from the resin with 95% TFA and 5% triisopropylsilane (TIPS) for 2 hours or 30% hexafluoroisopropanol (HFIP) in DCM. Next, the peptides were filtered into round bottom reaction flasks. The solvents were removed in vacuo, and the concentrates containing the peptides were precipitated and further triturated with ice cold diethyl ether (Et₂O). The identity of the synthesized peptides was confirmed by mass spectral analysis.

### 2. Peptide Purification

The synthesized peptides were purified by preparative reverse phase high performance liquid chromatography (RP-HPLC) using Waters 600 controller and pump system equipped with a Waters2489 UV/Visible detector and a Water Fraction Collector III (Waters Corp.). The purifications were typically performed on a Phenomenex Jupiter C18 10 micron 250×21.20 mm RP-HPLC column (Phenomenex, Inc.) with a flow rate of 20 mL/min. The acetonitrile/water (0.1% TFA) gradient was modified for each specific peptide based on hydrophobicity. The peptides were detected at two wavelengths, 228 nm and 280 nm, and the fractions were further analyzed by liquid chromatography mass spectrometry (LC-MS). Fractions containing peptide of adequate purity were pooled, flash frozen and lyophilized.

### 3. Peptide Characterization

The peptides were characterized by LC-MS (Agilent LC-MS TOF 6220 with 1200 series pump, auto handler and UV detection system). The LC separation was performed on a Phenomenex Jupiter C18 5 micron 250×2.00 mm column using a mobile phase of A (water+0.08% formic acid+0.02% trifluoroacetic acid) and B (acetonitrile+0.08% formic acid +0.02% trifluoroacetic acid). The general LC method had a gradient from 0-70% B over 12 min. Mass determination was achieved by electrospray ionization in positive mode. The purity of the peptides was determined by measuring the absorbance of UV light at 228 nm over the chromatogram.

### Example 1

### Thrombin-Activatable Procoagulant Compounds with PABC Self-Immolative Linker

Seven different peptides, designated Compound 1 to 7, were used in the experiments disclosed herein (TABLE 1). The sequence Ile-Val-Gly-Gly-Gln-Glu in Compounds 1 to 6 corresponds to the six N-terminal amino acid residues of the heavy chain of the FXa clotting factor. These compounds reproduce the coupling of a thrombin cleavable substrate and a self-immolative spacer to the N-terminus of a clotting factor or a fragment thereof, in this specific example, FX. Compound 7 corresponds to a synthetic procoagulant peptide fused to PABC and to a thrombin-cleavable substrate, and further including a linker and a scaffolding amino acid heterologous moiety (Cys) for attachment of half-life extending moieties such as PEG.

TABLE 1

| Com- | |
|---|---|
| pound | Structure |
| 1 | 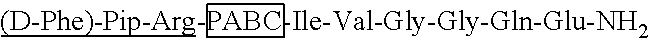 |
| 2 | (D-Phe)-Pip-Arg-Ile-Val-Gly-Gly-Gln-Glu-NH₂ |
| 3 | Ala-Leu-Arg-Pro-Arg-Ile-Val-Gly-Gly-Gln-Glu-NH₂ |
| 4 | 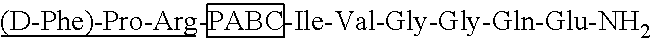 |
| 5 | 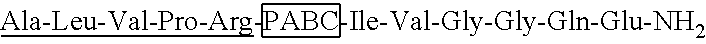 |
| 6 | Ala-Leu-Val-Pro-Arg-Ile-Val-Gly-Gly-Gln-Glu-NH₂ |
| 7 | 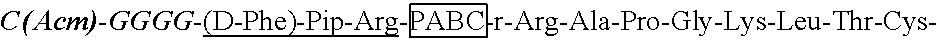 |
| | Leu-Ala-Ser-Tyr-Cys-Trp-Leu-Phe-Trp-Thr-Gly-Ile-Ala-NH₂ |
| | 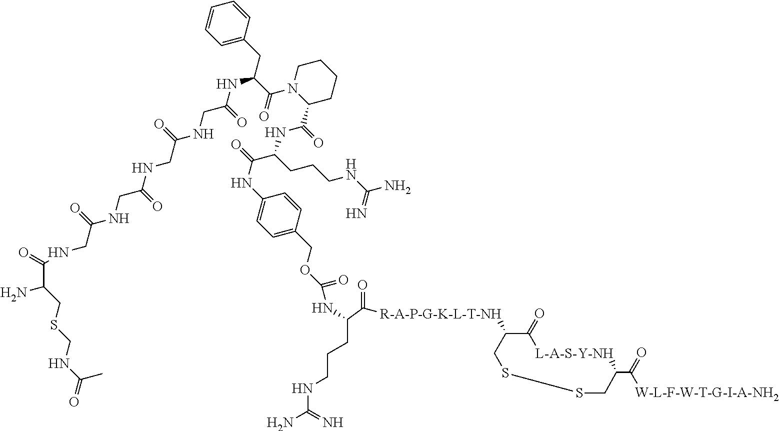 |

Pip is pipecolic acid. (ll-Phe) is D-Phenyl alanine. The sequences of the thrombin substrate are underlined. The location of the PABC self-immolative linker is indicated by a box.

### 1. Synthesis of PABC Peptides (Compound 1, 4, 5, and 7)

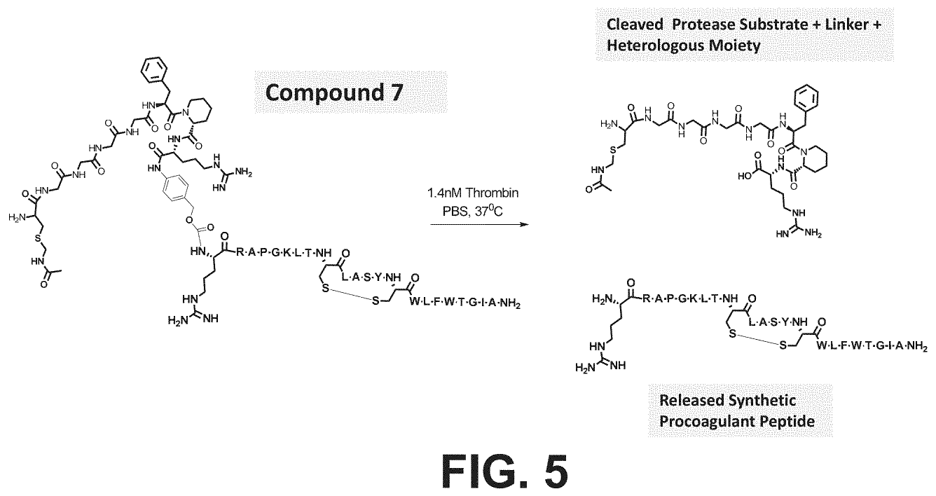

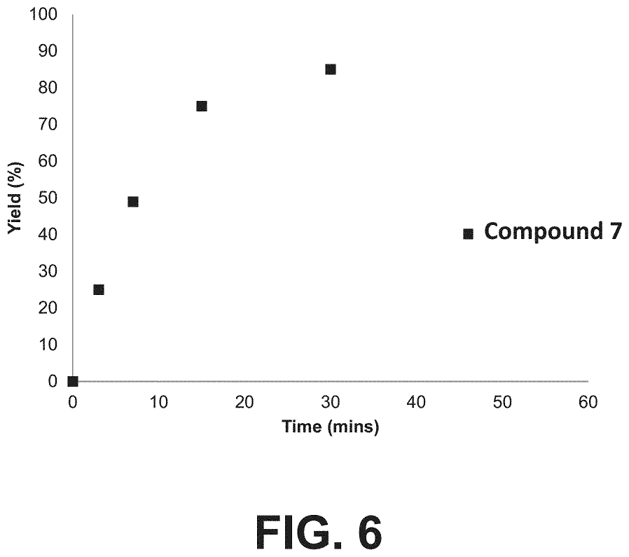

The synthesis process for Compound 7 is shown in FIG. 5 and FIG. 6, and explained in detailed below. The synthesis of Compounds 1, 4, and 5 was outsourced, and followed a similar synthesis procedure. Compounds 2, 3, 6 were synthesized as described in the Materials and Methods section, supra.

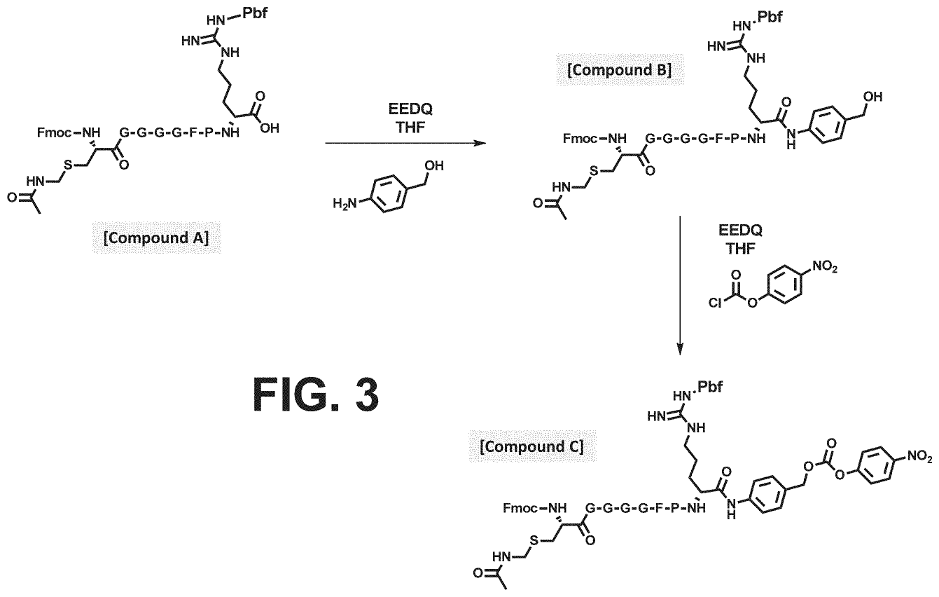

The first two steps in the synthesis of Compound 7 after cleavage from the resin are shown in FIG. 3:

### Compound A Synthesis

### Fmoc-Cys(Acm)-Gly-Gly-Gly-Gly-Dphe-Pip-Arg(Pbf)

The chemically synthesized and fully protected Compound A peptide (Fmoc-Cys(Acm)-Gly-Gly-Gly-Gly-Dphe-Pip-Arg(Pbf)) was cleaved from the NovaPEG TGT resin by 30% HFIP/DCM and filtered into a round bottom reaction flask. The solvents were removed in vacuo, and the concentrate containing the peptide was precipitated and further triturated with ice cold diethyl ether (Et2O). This material was directly used without further purification. ESI-MS m/z: 1309.51 (MH)⁺.

### Compound B Synthesis

Fmoc-Cys(Acm)-Gly-Gly-Gly-Gly-Dphe-Pip-Arg(Pbf)-PABOH (p-amino benzyl alcohol)

A stirred solution of Fmoc-Cys(Acm)-Gly-Gly-Gly-Gly-Dphe-Pip-Arg(Pbf) (Compound A) (268 mg, 0.2 mmol) and p-amino benzyl alcohol (28 mg, 1.1 equivalents) in THF (2 mL) at room temperature was treated with EEDQ (55.6 mg, 1.1 equivalents). After 16 hours, the mixture was evaporated to dryness, and the residue was triturated with ether. The resulting white solid product, comprising Compound B (Fmoc-Cys(Acm)-Gly-Gly-Gly-Gly-Dphe-Pip-Arg(Pbf)-PABOH (p-amino benzyl alcohol)) was collected by centrifugation and dried in vacuo (200 mg, 70%). ESI-MS m/z : 1414.61 (MH)⁺.

### Compound C Synthesis

### Fmoc-Cys(Acm)-Gly-Gly-Gly-Gly-Dphe-Pip-Arg(Pbf)-PABC-PNP

A stirred solution of Fmoc-Cys(Acm)-Gly-Gly-Gly-Gly-Dphe-Pip-Arg(Pbf)-PABOH (Compound B) (180 mg, 0.127 mmol) in dry THF (4 mL) and DCM (4mL) at room temperature was treated with PNP chloroformate (38.5 mg, 1.5 equivalents) and dry pyridine (15 mg, 1.5 equivalents). After 16 hours, the mixture was concentrated to 1 mL, and the product was precipitated and triturated with cold ether. The resulting white solid product, comprising Compound C (Fmoc-Cys(Acm)-Gly-Gly-Gly-Gly-Dphe-Pip-Arg(Pbf)-PABC-PNP) was collected by centrifugation and dried in vacuo (150 mg, 75%). ESI-MS m/z: 1579.61 (MH)⁺.

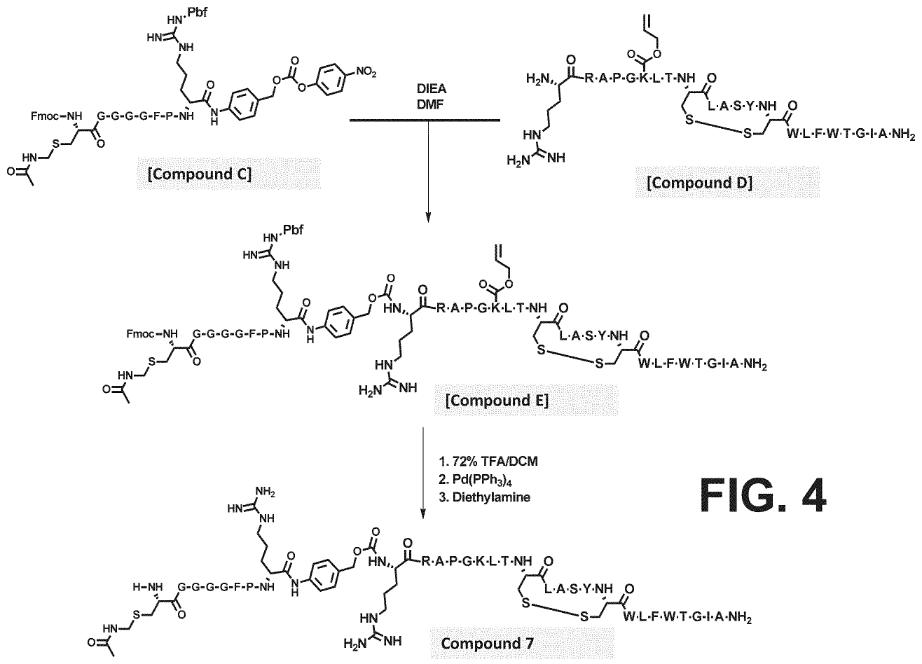

The remaining steps in the synthesis of Compound 7, comprising the conjugation of the protease-cleavable substrate/self-immolative spacer to the synthetic procoagulant peptide are depicted in FIG. 4.

### Compound D Synthesis

rRAPGK(Alloc)LTCLASYCWLFWTGIA-NH₂ (disulfide)

The linear peptide was synthesized on NovaPEG Rink Amide resin (0.2 mmol) as described in the general method. The Cys-Cys disulfidic bond was formed by stirring the crude peptide in 50% DMSO/H20 overnight at 37° C. 35 mg of peptide was obtained after purification by preparative HPLC. ESI-MS m/z: 1298.17 (MH₂)²⁺, 865.78 (MH₃)³⁺.

### Compound E Synthesis

Fmoc-Cys(Acm)-Gly-Gly-Gly-Gly-Dphe-Pip-Arg(Pbf)-PABC-rRAPGK(Alloc)LTCLA SYCWLFWTGIA-NH₂ (disulfide)

Fmoc-Cys(Acm)-Gly-Gly-Gly-Gly-Dphe-Pip-Arg(Pbf)-PABC-PNP (Compound C) (12.5 mg, 0.008 mmol) and rRAPGK(Alloc)LTCLASYCWLFWTGIA (Compound D) (30 mg, 0.011 mmol) in DMF (1 mL) at room temperature were treated with DIEA (6.5 μL, 5 equivalents). The mixture was allowed to stand in the dark overnight. The crude product was precipitated, and triturated with cold ether. The resulting crude product was collect by centrifugation, dried in vacuo, and used for next step without further purification. ESI-MS m/z: 2018.32 (MH₂)²⁺, 1345.86 (MH₃)³⁺.

### Compound 7 Synthesis

### Step 1

Fmoc-Cys(Acm)-Gly-Gly-Gly-Gly-Dphe-Pip-Arg-PABC-rRAPGK(Alloc)LTCLASYCWLFWTGIA-NH₂ (disulfide)

Pbf deprotection of Fmoc-Cys(Acm)-Gly-Gly-Gly-Gly-Dphe-Pip-Arg(Pbf)-PABC-rRAPGK(Alloc)LTCLA SYCWLFWTGIA from the previous step was carried out in 1 mL of solvent mixture (72% TFA, 5% DMF, 5% H₂O, 18% DCM) for 75 minutes. Since the PABC linker was unstable under this condition, aliquots were taken at various time points to monitor progress of the reaction. At 75 minutes, cold ether (50 mL) was added to stop the reaction. The resulting solid was purified by preparative HPLC, to give a white powder (8 mg, 25% for 2 steps). ESI-MS m/z: 1261.83 (MH3)³⁺.

### Step 2

Fmoc-Cys(Acm)-Gly-Gly-Gly-Gly-Dphe-Pip-Arg-PABC-rRAPGKLTCLA SYCWLFWTGIA-NH₂ (disulfide)

Fmoc-Cys(Acm)-Gly-Gly-Gly-Gly-Dphe-Pip-Arg-PABC-rRAPGK(Alloc)LTCLASYCWLFWTGIA (8 mg, 0.002 mmol) in MeOH/Dioxane (1:1, 180 μL) under N₂ at room temperature was treated with Pd(PPh₃)₄ (0.0002 mmol, 0.1 equivalents, 20 μL of a THF solution of Pd(PPh₃)₄ (23 mg/mL), followed by PhSiH₃ (0.01 mmol, 5 equivalents). After 20 minutes, the crude mixture was precipitated and triturated with cold ether. The resulting crude product was used for the next step without purification. ESI-MS m/z: 1233.82 (MH₃)³⁺.

### Step 3

Cys(Acm)-Gly-Gly-Gly-Gly-Dphe-Pip-Arg-PABC-rRAPGKLTCLASYCWLFWTGIA-NH₂ (disulfide) (Compound 7)

Fmoc deprotection of Fmoc-Cys(Acm)-Gly-Gly-Gly-Gly-Dphe-Pip-Arg-PABC-rRAPGKLTCLASYCWLFWTGIA from the previous step was carried out in DMSO (200 μL) with Et₂NH (50 μL, excess). After 20 minutes, the reaction was complete and the mixture was purified by preparative HPLC, to give a white powder SYN 4018 (0.83 mg, 12% over 2 steps). ESI-MS m/z: 1159.80 (MH₃)³⁺, 870.09 (MH₄)⁴⁺.

### 2. Thrombin Cleavage of Compound 7

FIG. 5 depicts the cleavage of Compound 7 by thrombin. Upon cleavage with 1.4nM thrombin in PBS, the clean synthetic procoagulant peptide is released, as well as the portion of the molecule comprising the thrombin substrate, the GGGG linker, and the N-terminal cysteine. To conduct the reaction, 21 μL of peptide (0.24 mM) in water was added to 476.5 μL PBS. The mixture was incubated at 37° C. for 30min, followed by 2.5 μL of thrombin (278 nM, 10 μg/mL), giving the following approximate initial concentrations:thrombin=1.4 nM, peptide=10 μM. The mixture was incubated at 37° C. Aliquots (60 μL) at various time points were quenched with 1 μL of hirudin (10 μM) and injected into the HPLC (C-18 column, CH₃CN/H₂O, 0 to 70% over 12 minutes, 60° C. 0.5 mL/min, λ=280 nm). The kinetics of the cleavage of Compound 7 is shown in FIG. 6. Compound 7 was cleaved rapidly by 1.4 nm thrombin. Approximately 90% of Compound 7 was cleaved after 30 minutes.

Compound 7 was also cleaved completely during the course of a TGA assay (FIG.

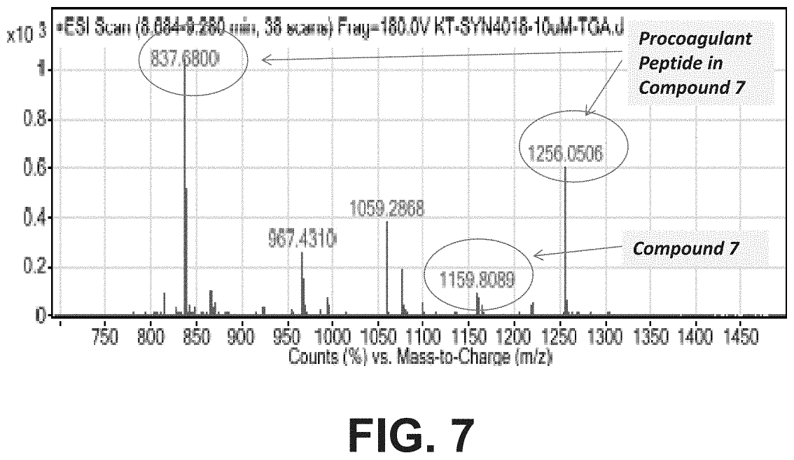

7). Plasma at the end of TGA assay for Compound 7 was transferred into cold CH₃CN (1 mL) and centrifuged at 13k rpm for 10 min. Supernatant (1.1 mL) was transferred to a new vial and dried by speedvac. The resulting solid was reconstituted with 30 μL of H₂O and injected into the HPLC for analysis. FIG. 7 shows peaks corresponding to the procoagulant peptide in Compound 7, indicating that Compound 7 was completely cleaved in the course of the TGA assay.

### 3. Thrombin Cleavage of Compounds 1, 2, and 3

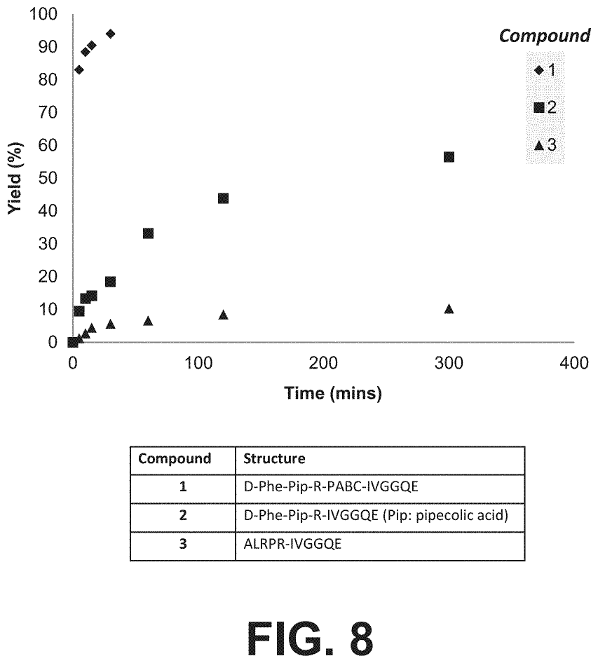

FIG. 8 depicts the cleavage of Compounds 1, 2 and 3 by 14 nM thrombin. These compounds, as discussed above, comprise the six N-terminal amino acid residues of the heavy chain of the FXa clotting factor, and function as a model to show the applicability of the procoagulant compound design disclosed herein to clotting factors.

In this specific example, 50 μL of peptide (1 mM) in water was added to 900 μL PBS, followed by 50 μL of thrombin (278 nM, 10 μg/mL), giving the following approximate initial concentrations: thrombin=14 nM, peptide=50 μM. The mixture was incubated at room temperature. Aliquots (95 μL) at various time points were quenched with 5 μL of hirudin (2 μM) and injected into the HPLC (C-18 column, CH₃CN/H₂O, 0 to 70% over 12 minutes, 60° C. 0.5 mL/min, λ=280 nm). The decreases of peptide peak areas were used to calculate yield.

Compared to Compounds 2 and 3, the construct incorporating the thrombin-cleavable synthetic substrate D-Phe-Pip-Arg (SEQ ID NO: 21) and the self-immolative spacer PABC (Compound 1) was a better substrate for thrombin. The incorporation of PABC to Compound 1 led to at least 10-fold increase in cleavage rate compared to that of Compound 2.

4. Thrombin cleavage of Compounds 1, 4, 5 and 6

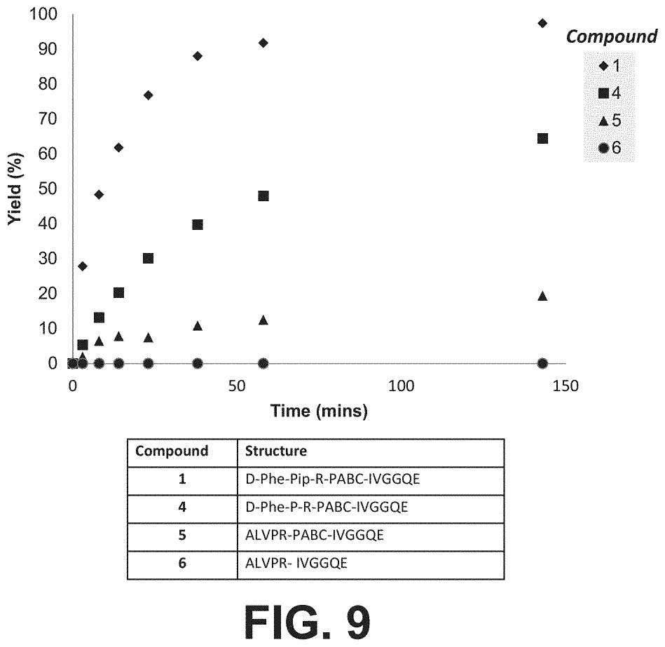

FIG. 9 depicts the cleavage of Compounds 1, 4, 5 and 6 by 1.4 nM thrombin. Compounds 1, 4 and 5 incorporate PABC and different thrombin-cleavable substrates.

50 μL of peptide (1 mM) in water was added to 900 μL PBS. The mixture was incubated at 37° C. for 30 min, followed by 50 μL of thrombin (27.8 nM, 1 μg/mL), giving the following approximate initial concentrations: thrombin=1.4 nM, peptide=50 μM. The mixture was incubated at 37° C. Aliquots (95 μL) at various time points were quenched with 5 μL of hirudin (2 μM) and injected into the HPLC (C-18 column, CH₃CN/H₂O, 0 to 70% over 12 minutes, 60° C. 0.5 mL/min, λ=280 nm). The decreases of peptide peak areas were used to calculate yield.

Compound 1 was a better substrate for thrombin than Compounds 4 and 5. At 1.4 nM, a physiological relevant concentration of thrombin, 30% of Compound 1 was quickly cleaved and released. In contrast, thrombin-mediated release of peptide IVGGQE (SEQ ID NO: 85) from Compound 6 without PABC linker was not observed.

### Example 2

### Thrombin-activatable FX with PABC Self-Immolative Linker

Peptide synthesis method equivalents to those described above, standard recombinant protein production methods, and standard chemical conjugation techniques are used to generate the procoagulant compound described in this example.

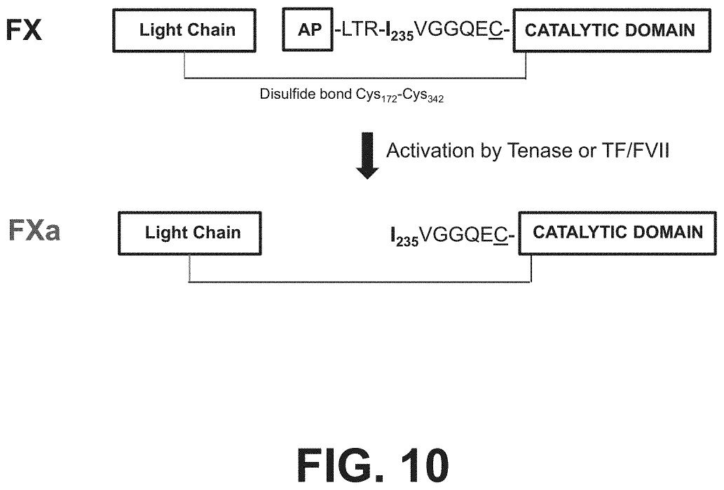

Factor X consists of two polypeptide chains linked by a disulfide bridge (Cys172-Cys342): the 139 amino acid light chain in composed of the Gla domain and the two EGFs; the 306 amino acid heavy chain is composed of the activation peptide joined to the catalytic domain. The activation of factor X requires proteolytic cleavage between the activation peptide and the catalytic domain. The tensase complex and the FVIIa-TF complex perform this cleavage between the Arg234 and Ile235 residues (FIG. 10). As in all serine proteases, the N-terminal residues of the catalytic chain of activated factor X are involved in the enzymatic activity. The generated N-terminal Ile235 in particular plays a fundamental role in the catalytic mechanism of the enzyme.

PCT Publ. No. WO 2004/005347 proposed replacement of the native site for activation by the tenase complex with a site for cleavage by thrombin. However, since the efficiency of cleavage is conditioned by the nature of the amino acids framing the cleavage site, these thrombin-activatable FX (TA-FX) analogs suffered from slow activation and undesired efficacy. It is therefore desirable to have other TA-FX analogs which would exhibit faster cleavage kinetics.

The present disclosure provides TA-FX analogs comprising a synthetic thrombin substrate and a self-immolative spacer (e.g., PABC) linked to FXa (FIG. 11). After proteolytic cleavage of the thrombin substrate (D-PhePipArg) and 1,6 spontaneous fragmentation, the natural sequence of FXa is released (FIG. 10). The TA-FX is generated semi-synthetically using native chemical ligation chemistry. This process involves the reaction of a recombinantly produced FX fragment containing an N terminal cysteine residue 241 on the catalytic domain (CysFX) with a synthetically produced thioester peptide to generate a native amide bond at the linkage site. To generate the CysFX protein, the sequence comprising the six N-terminal amino acid residues of the heavy chain IVGGQE (SEQ ID NO: 85) is truncated from the FX; and the native site for activation by tensase complex is replaced with a cleavage site, e.g., for cleavage by PCS.

### Example 3

### Thrombin-activatable FVII with PABC Self-Immolative Linker

Peptide synthesis method equivalent to those described above, standard recombinant protein production methods, and standard chemical conjugation techniques are used to generate the procoagulant compounds described in this example.

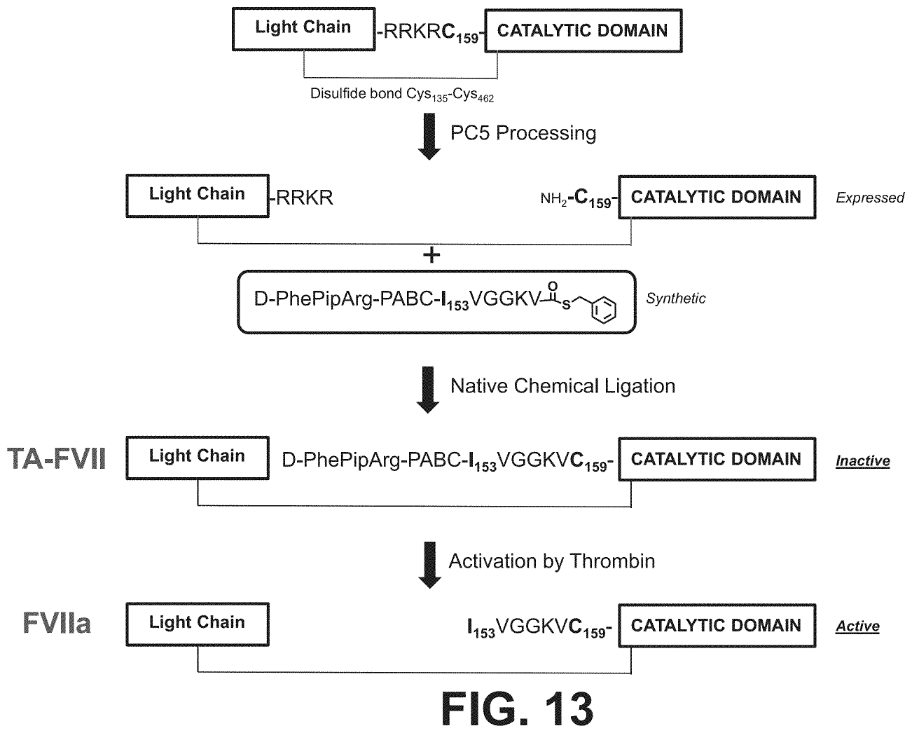

The present disclosure provides thrombin-activatable FVII (TA-FVII) analogs comprising a synthetic thrombin substrate and a self-immolative spacer (e.g., PABC) linked to FVIIa (FIG. 13). After proteolytic cleavage of the thrombin substrate (D-PhePipArg) and 1,6 spontaneous fragmentation, the natural sequence of FVIIa is released (FIG. 10). The TA-FVII is generated semi-synthetically using native chemical ligation chemistry. This process involves the reaction of a recombinantly produced FVII fragment containing an N terminal cysteine residue 159 on the catalytic domain (CysFVII) with a synthetically produced thioester peptide to generate a native amide bond at the linkage site. To generate the CysFVII protein, the sequence comprising the six N-terminal amino acid residues of the heavy chain IVGGKV (SEQ ID NO: 83) is truncated from the FVII; and the native site for activation by FXa is replaced with a cleavage site, e.g., for cleavage by PC5.

### Example 4

### Thrombin activatable FVII-186 with SUMO Cleavage Site

For cloning of FVII-186, synthesis of the DNA sequence comprising nucleotides from the HindIII site to the EcoRI site of FVII-186 (Table 2) was outsourced. The DNA was subcloned into the HindIII/EcoRI sites of pcDNA.

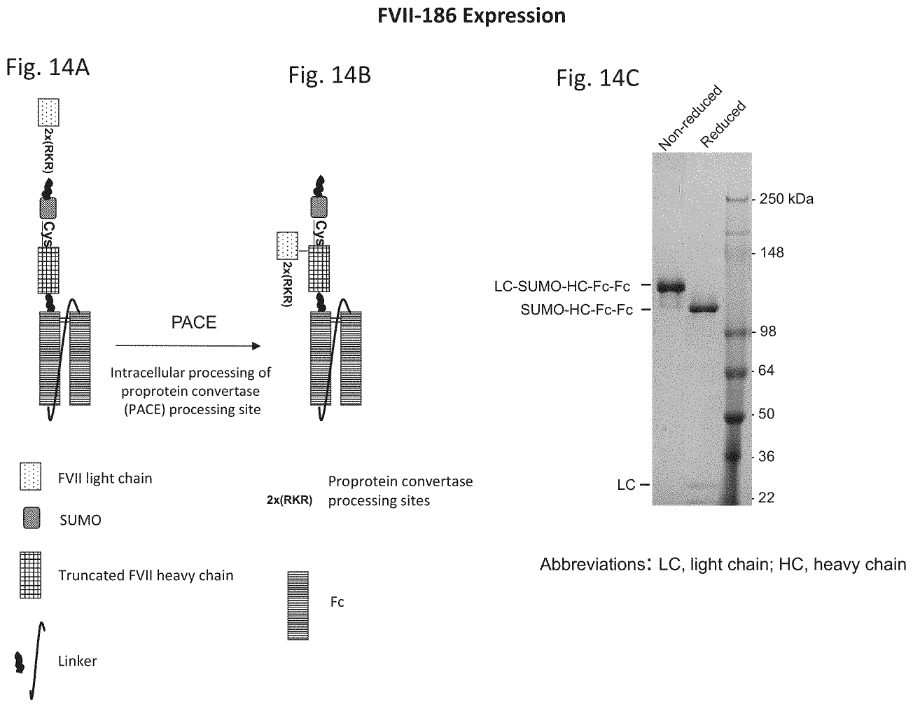

To transiently express FVII-186, HEK-293-F cells were grown in suspension in FREESTYLE® media (Invitrogen) supplemented with vitamin K3 (Sigma Aldrich, St. Louis, Mo.) to 2 μg/liter (growth media) as suspension cells at 37° C./10% CO₂. Cells were subcultured every three to four days by seeding at cell density of 5×10⁵ cells/ml. Twenty-four hours prior to transfection, cells were seeded at a density of 7×10⁵ cells/ml in growth media. On the day of transfection, a transfection solution was made with a volume equal to 5% of the total volume of the cell culture to be transfected. In the transfection solution, DNA was added (final concentration 20 mg/L) to a freshly made solution of PEI (60 mg/L) in growth media. The solution was swirled for 30 seconds and incubated for five minutes at room temperature before adding directly to the cell culture. Four hours later a volume equal to the cell culture volume of OPTICHO™ (Invitrogen) supplemented with vitamin K3 and 200 mM L-glutamine was added to the cells. The cell culture was allowed to grow as shown above and daily media samples were taken to assess protein expression. On the day of harvest, the cells were spun down, and the media filtered in preparation for protein purification or protein analysis by protein A pulldown. For expression of FVII-186, a plasmid encoding FVII-186 was contransfected with a plasmid encoding the proprotein convertase PACE to ensure intracellular processing and cleavage of the proprotein convertase cleavage sites (2X(RKR) SEQ ID NO: 88) in the linker connecting the FVII light chain to SUMO (FIG. 14).

To purify FVII-186, conditioned medium was loaded onto a 25-mL column of Q SEPHAROSE® Fast Flow (GE HealthCare Life Sciences) after adjustment of pH to 7.4 with 2.0 M Tris, pH 8.0. Column was washed with 10 mM MES, 50 mM NaCl, pH 6.5. The protein was eluted with 10 mM MES, 100 mM NaCl, 20 mM CaCl₂, pH 6.5. The fractions containing FVII-186 were pooled and loaded onto a 25-mL column of rhFcRn-sepharose after adjustment of pH to 6.2 with 0.5 M MES, pH 5.5. After washing with 50 mM MES, 100 mM NaCl, pH 6.2, the bound material was eluted with 10 mM Tris, 250 mM NaCl, pH 8.0 and analyzed with SDS-PAGE.

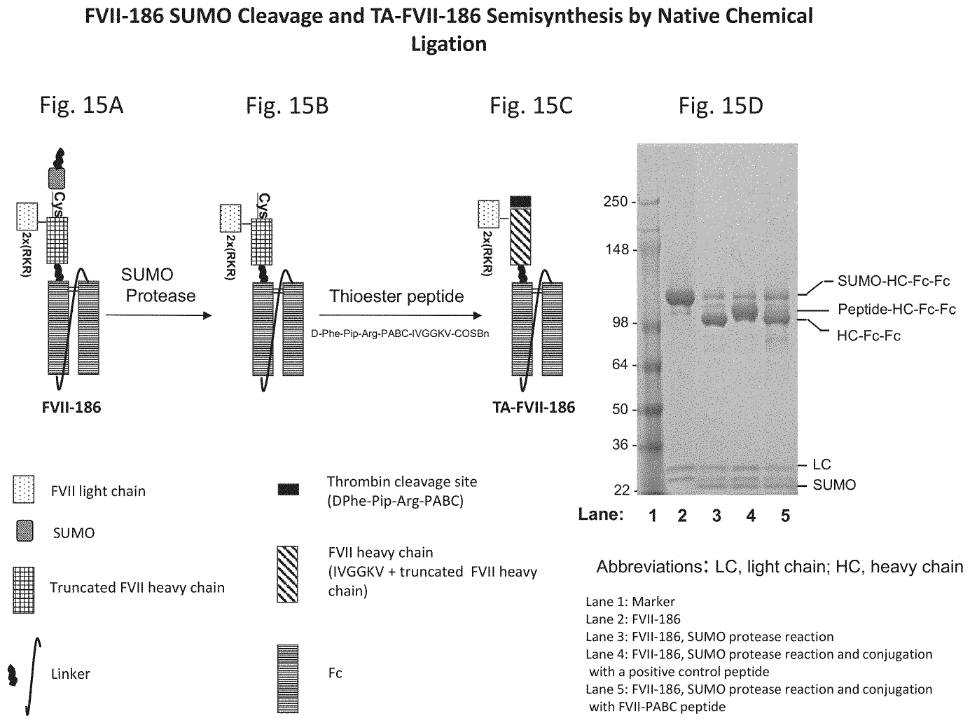

FVII-186 was cleaved by a SUMO protease as follows. FVII-186 (0.83 mg/mL, 10 μL) was incubated with 10 μL of 100 mM HEPES, 20 mM CaCl₂, 0.004% Tween 80 containing 0.4 mM oxidized Glutathione (GSSG), 20 mM Glutathione (GSH), 0.2 U/μL SUMO protease (Invitrogen Cat. No.12588-018) for 48 hours at room temperature. Reducing SDS-PAGE (FIG. 15, lane 3) showed almost complete conversion of FVII-186 to the desired FVIIHC.

For SUMO protease cleavage of FVII-186 and native chemical ligation with a thioester peptide, FVII-186 (0.83 mg/mL, 10 μL) was incubated with 10 μL of 100 mM HEPES, 20 mM CaCl₂, 0.004% Tween 80 containing 0.4 mM SYN470 as a positive control peptide, 0.4 mM GSSG, 20 mM GSH, 0.2 U/μL SUMO protease (Invitrogen Cat. No.12588-018) for 48 hours at room temperature. Reducing SDS-PAGE (FIG. 15, lane 4) showed complete disappearance of the FVIIHC band and a single new band as the conjugate of the positive peptide control and the FVIIHC.

In order to synthesize Thrombin Activatable FVII-186 (TA-FVII-186), FVII-186 (0.83 mg/mL, 200 μL) was incubated with 200 μL of 100 mM HEPES, 20 mM CaCl₂, 0.004% Tween 80 containing 0.4 mM FVII-PABC peptide (i.e., Biotin-Pra-GGGG-D-Phe-Pip-Arg-PABC-IVGGKV-COSBn) (SEQ ID NO: 79), 0.4 mM GSSG, 20 mM GSH, 0.2 U/μL SUMO protease (Invitrogen Cat. No.12588-018) for 48 hours at room temperature and analyzed by reducing SDS-PAGE (FIG. 15, lane 5). Reaction mixture was placed in a 0.5 mL dialysis cassette with 10k MWCO and dialyzed against 1 L of 10 mM Tris, 250 mM NaCl, pH 8.0 containing 0.4 mM GSSG, 2 mM GSH for 24 hours at 4° C. The conjugate was further purified by rhFcRn- sepharose column as described.

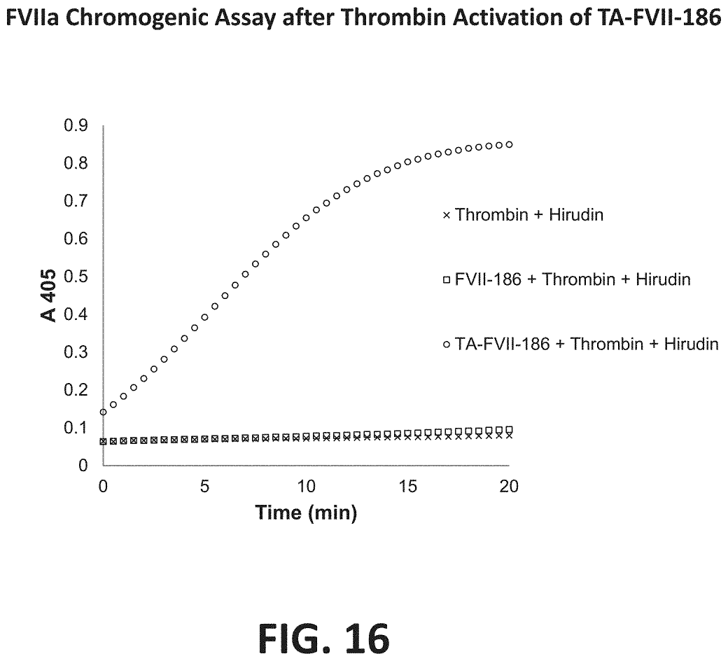

FVIIa Chromogenic assay was performed after Thrombin cleavage and activation of TA-FVII-186 (FIG. 16). This assay measures the FX activation activity by measuring the ability of FVIIa to activate FX, as determined by measuring levels of a chromogenic substrate that is cleaved by activated FX (FXa). TA-FVII-186 (200 nM) was activated with Thrombin (140 nM) for 20 minutes at 37° C. Hirudin was added to quench Thrombin. sTF-PL mixture (A STACLOT® FVII-rTF kit), FX, and PEFACHROME® FXa substrate were added and reaction was monitored by measuring absorbance at 405 nm. FVII-186 missing the six N-terminal amino acids was not active in the presence of thrombin. Only TA-FVII-186 with a thrombin cleavage site connected to the complete heavy chain FVII (which includes FVIIa—PABC peptide) showed activity after thrombin cleavage. The resulted activity demonstrated that the FVIIa-PABC peptide was successfully conjugated to the N-terminal cysteine residue of the truncated heavy chain of FVIIa, the crucial N-terminal isoleucine residue was generated upon cleavage by thrombin, and the formed protein had the essential structure for activity.

### Example 5

### Thrombin Activatable FX-011 with PACE Cleavage Site

For cloning of FX-011, synthesis of the DNA sequence comprising nucleotides from the Hindlll site to the Notl site of FX-011 (Table 4) was outsourced. The DNA was subcloned into the HindIII/NotI sites of pcDNA.

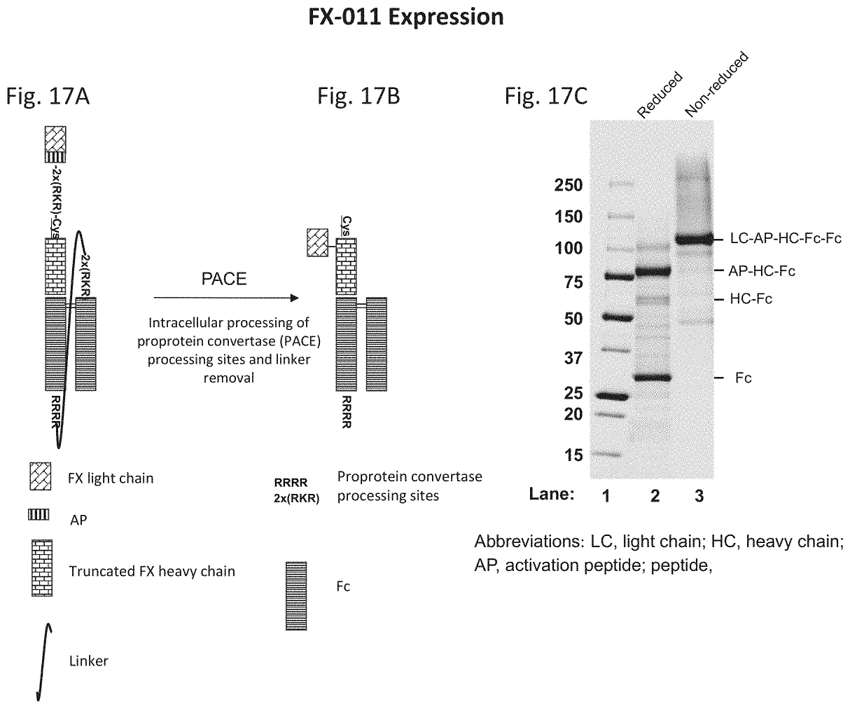

For transient expression of FX-011, HEK-293-F cells were transfected essentially as described above to obtain expression of FX-011. A plasmid encoding FX-011 was cotransfected with a plasmid encoding the proprotein convertase PACE (20%) to ensure intracellular processing and cleavage of the proprotein convertase cleavage sites in the linkers and removal of linkers (FIG. 17) In order to analyze the protein from transient transfections, conditioned media were subjected to protein A immunoprecipitation. Briefly, cell culture supernatant was mixed with approximately 50 μl of protein A-Sepharose 50% slurry and incubated at 4° C. with rocking for 1 hour, then centrifuged to pellet the protein A beads. Beads were washed twice by resuspending in 1 ml of PBS, spinning and aspirating. The beads were resuspended with SDS-PAGE buffer under reducing or nonreducing conditions, heated for 5 minutes at 95° C., spun down, loaded on SDS-PAGE gels, and run according to standard protocols. Under non-reducing conditions, 1 band with the expected molecular weight for the FX-011 was observed (FIG. 17, lane 3). Under reducing conditions, 3 major bands were observed representing the incompletely processed activation peptide-heavy chain FX-Fc subunit, the desired heavy chain FX-Fc subunit, and the Fc subunit (FIG. 17, lane 2). Proteins were transferred onto a cellulose membrane and the band corresponding to heavy chain FX-Fc subunit was collected and analyzed. N-terminal sequencing confirmed the existing N-terminal cysteine (Cys) residue as expected after cleavage by PACE.

For purification of FX-011, conditioned medium (200 mL) was concentrated to 10 mL by 15 mL centrifugal filter units 30,000 MWCO (catalog # UFC 903008). After adjustment of pH to 6.2 with 0.5 M MES, pH 5.5, the concentrated medium was loaded onto a 0.5 mL rhFcRn-sepharose resin bed equilibrated with 50 mM MES, 100 mM NaCl, pH 6.2 buffer. After washing with 50 mM MES, 100 mM NaCl, pH 6.2, the bound material was eluted with 10 mM Tris, 250 mM NaCl, pH 8.0. Before conjugation, FX-011 was transferred to a 20 mM HEPES, 500 mM NaCl, 5 mM CaCl₂, pH 7.4 buffer by dialysis.

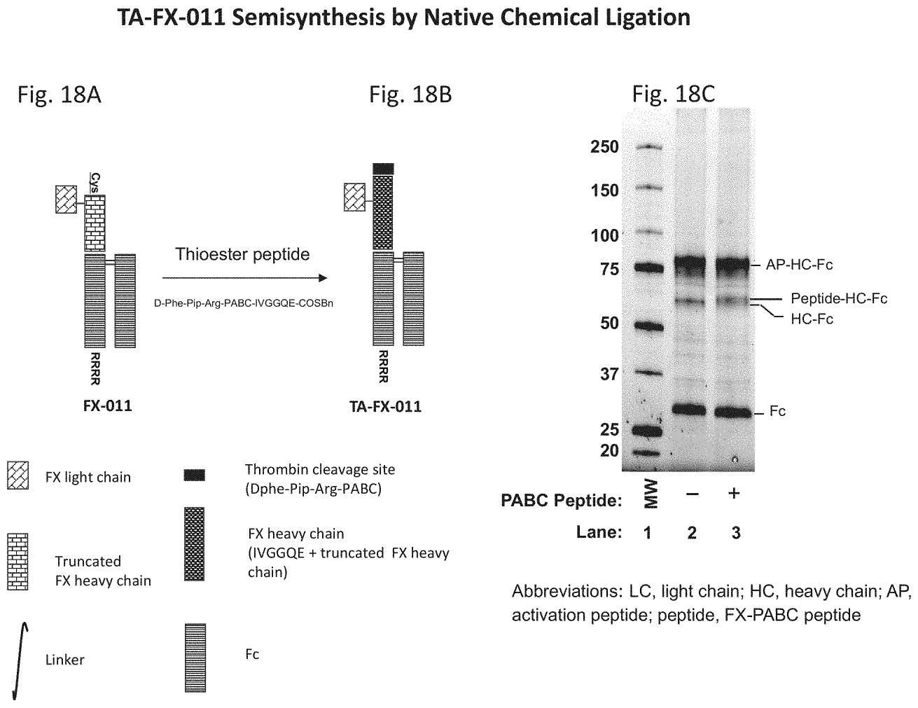

For semisynthesis of Thrombin Activatable FX-011 (TA-FX-011) by native chemical ligation with a thioester peptide, FX-011 (0.5 mg/mL) was incubated with 0.5 mM FX-PABC peptide (i.e., GG-D-Phe-Pip-Arg-PABC-IVGGQE-COSBn) (SEQ ID No_) and 20 mM sodium 2-sulfanylethanesulfonate (MESNA) in 20 mM HEPES, 500 mM NaCl, 5 mM CaCl₂, pH 7.4 buffer for 16 hours at room temperature. Reaction was analyzed by SDS-PAGE gel (FIG. 18, lane 3). Excess peptides and MESNA were removed by gel filtration. The pooled fractions containing TA-FX-011 were placed in a 0.5 mL dialysis cassette with 10k MWCO and dialyzed against 1 L of 20 mM HEPES, 500 mM NaCl, 5 mM CaCl₂, pH 7.4 for 24 hours at 4° C.

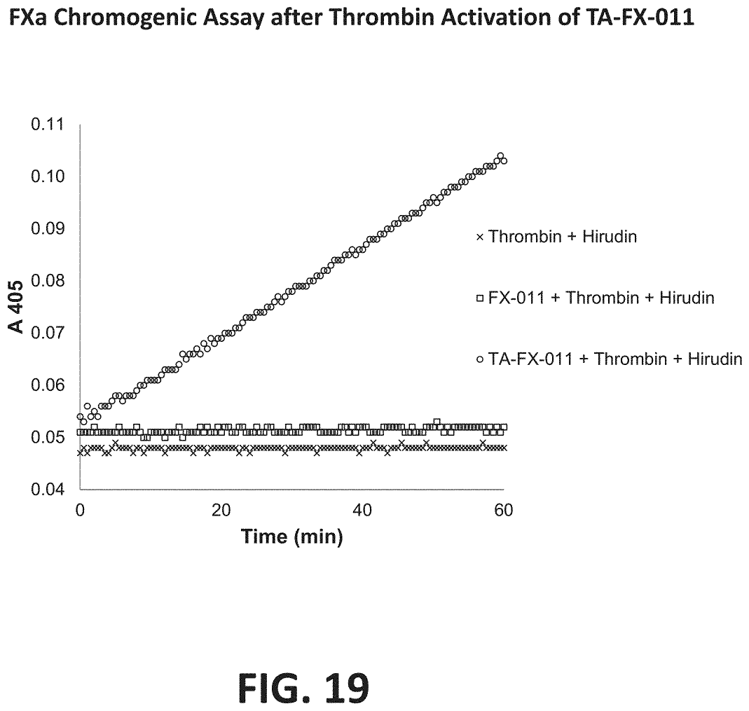

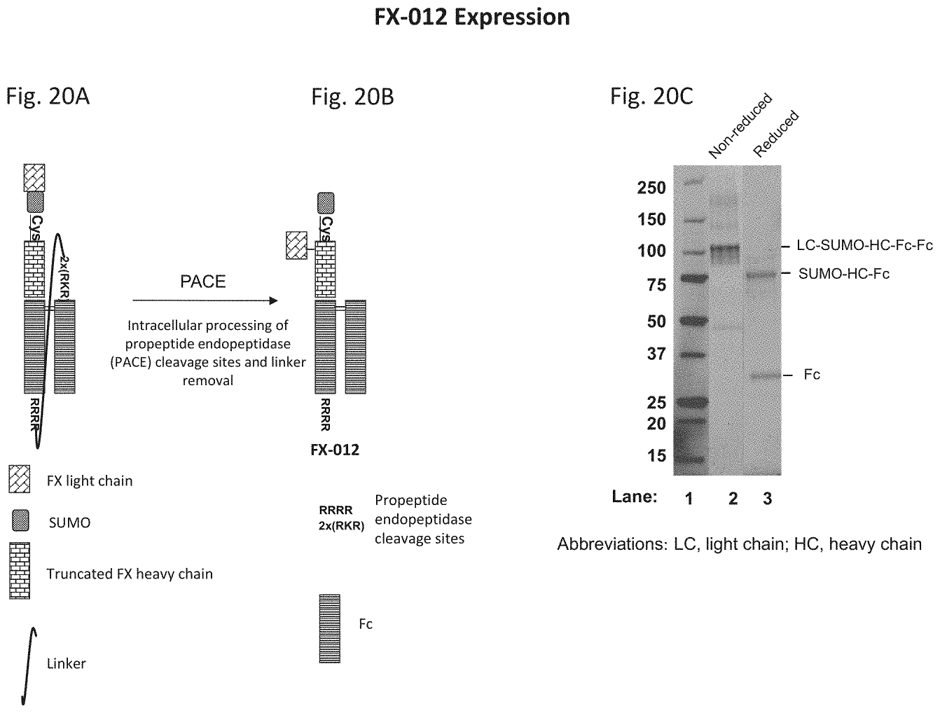

FXa chromogenic assay was performed after Thrombin cleavage of TA-FX-011 (FIG. 19). TA-FX-011 (200 nM) was activated with Thrombin (140 nM) for 20 minutes at 37° C. Hirudin was added to quench Thrombin. FXa substrate was added and reaction was monitored by measuring absorbance at 405 nm. FX-011 missing the six N-terminal amino acids was not active in the presence of thrombin. Only TA-FX-011 with a thrombin cleavage site connected to the complete heavy chain FX (which includes FXa-PABC peptpide) showed activity after thrombin cleavage. The resulted activity demonstrated that the FX PABC peptide was successfully conjugated to the N-terminal cysteine residue of the truncated heavy chain of FX, the crucial N-terminal isoleucine residue was generated upon cleavage by thrombin, and the formed protein had the essential structure for activity.

### Example 6

### Thrombin Activatable FX-012 with SUMO Cleavage Site

For cloning of FX-012, synthesis of the DNA sequence comprising nucleotides from the Hindlll site to the NotI site of FX-012 (Table 6) was outsourced. The DNA was subcloned into the HindIII/NotI sites of pcDNA.

Transient expression and protein purification of FX-012 was essentially as described for FVII-186. Under non-reducing conditions, the major band with the expected molecular weight for the FX-012 was observed (FIG. 20, lane 2). Under reducing conditions, 2 major bands were observed representing the desired SUMO-heavy chain FX-Fc subunit and the Fc subunit (FIG. 20, lane 3). The LC band was not visible.

FX-012 was cleaved by a SUMO protease as follows. FX-012 (0.35 mg/mL) was incubated with 0.1 U/μL SUMO protease (Invitrogen Cat. No.12588-018), 20 mM GSH in 50 mM HEPES, 10 mM CaCl₂, pH 7.4 buffer for 24 hours at room temperature. Reducing SDS-PAGE (FIG. 21, lane 2) showed almost complete conversion of FX-012 to the desired FXHC-Fc.

For SUMO protease cleavage of FX-012 and native chemical ligation with a thioester peptide, FX-012 (0.35 mg/mL) was incubated with 0.4 mM SYN470 as a positive control peptide, 0.1 U/μL SUMO protease (Invitrogen Cat. No.12588-018), 20 mM GSH in 50 mM HEPES, 10 mM CaCl₂, pH 7.4 buffer for 24 hours at room temperature. Reducing SDS-PAGE (FIG. 21, lane 4) showed complete disappearance of the FXHC-Fc band and a single new band as the conjugate of the positive peptide control and the FXHC-Fc.

For semisynthesis of Thrombin Activatable FX-012 (TA-FX-012) by native chemical ligation with a thioester peptide, FX-012 (0.35 mg/mL) was incubated with 0.4 mM FX-PABC peptide (i.e., D-Phe-Pip-Arg-PABC-IVGGQE-COSBn) (SEQ ID NO: 90), 0.1 U/μL SUMO protease (Invitrogen Cat. No.12588-018), 20 mM GSH in 50 mM HEPES, 10 mM CaCl₂, pH 7.4 buffer for 24 hours at room temperature. Reducing SDS-PAGE (FIG. 21, lane 3) showed a new band as the desired conjugate of the FX-PABC peptide and the FXHC-Fc. Reaction mixture was placed in a 0.5 mL dialysis cassette with 10 k MWCO and dialyzed against 1 L of 10 mM Tris, 250 mM NaCl, pH 8.0 containing 0.4 mM GSSG, 2 mM GSH for 24 hours at 4° C. The conjugate was further purified by rhFcRn- sepharose column as described.

FXa chromogenic assay was performed after Thrombin cleavage of TA-FX-012 (FIG. 22). TA-FX-012 (200 nM) was activated with Thrombin (140 nM) for 20 minutes at 37° C. Hirudin was added to quench Thrombin. FXa substrate was added and reaction was monitored by measuring absorbance at 405 nm. FX-012 missing the six N-terminal amino acids was not active in the presence of thrombin. Only TA-FX-012 with a thrombin cleavage site connected to the complete heavy chain FX (which includes FXa-PABC peptide) showed activity after thrombin cleavage. The resulted activity demonstrated that the FX PABC peptide was successfully conjugated to the N-terminal cysteine residue of the truncated heavy chain of FX, the crucial N-terminal isoleucine residue was generated upon cleavage by thrombin, and the formed protein had the essential structure for activity.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

### TABLES

TABLE 2
DNA sequence of FVII-186 (SEQ ID NO: 65)

| | |
|---|---|
| 1 | AAGCTTGCCG CCACCATGGT CTCCCAGGCC CTCAGGCTCC TCTGCCTTCT GCTTGGGCTT |
| | TTCGAACGGC GGTGGTACCA GAGGGTCCGG GAGTCCGAGG AGACGGAAGA CGAACCCGAA |
| 61 | CAGGGCTGCC TGGCTGCAGT CTTCGTAACC CAGGAGGAAG CCCACGGCGT CCTGCACCGG |
| | GTCCCGACGG ACCGACGTCA GAAGCATTGG GTCCTCCTTC GGGTGCCGCA GGACGTGGCC |
| 121 | CGCCGGCGCG CCAACGCGTT CCTGGAGGAG CTGCGGCCGG GCTCCCTGGA GAGGGAGTGC |
| | GCGGCCGCGC GGTTGCGCAA GGACCTCCTC GACGCCGGCC CGAGGGACCT CTCCCTCACG |
| 181 | AAGGAGGAGC AGTGCTCCTT CGAGGAGGCC CGGGAGATCT TCAAGGACGC GGAGAGGACG |
| | TTCCTCCTCG TCACGAGGAA GCTCCTCCGG GCCCTCTAGA AGTTCCTGCG CCTCTCCTGC |
| 241 | AAGCTGTTCT GGATTTCTTA CAGTGATGGG GACCAGTGTG CCTCAAGTCC ATGCCAGAAT |
| | TTCGACAAGA CCTAAAGAAT GTCACTACCC CTGGTCACAC GGAGTTCAGG TACGGTCTTA |
| 301 | GGGGGCTCCT GCAAGGACCA GCTCCAGTCC TATATCTGCT TCTGCCTCCC TGCCTTCGAG |
| | CCCCCGAGGA CGTTCCTGGT CGAGGTCAGG ATATAGACGA AGACGGAGGG ACGGAAGCTC |
| 361 | GGCCGGAACT GTGAGACGCA CAAGGATGAC CAGCTGATCT GTGTGAACGA GAACGGCGGC |
| | CCGGCCTTGA CACTCTGCGT GTTCCTACTG GTCGACTAGA CACACTTGCT CTTGCCGCCG |
| 421 | TGTGAGCAGT ACTGCAGTGA CCACACGGGC ACCAAGCGCT CCTGTCGGTG CCACGAGGGG |
| | ACACTCGTCA TGACGTCACT GGTGTGCCCG TGGTTCGCGA GGACAGCCAC GGTGCTCCCC |
| 481 | TACTCTCTGC TGGCAGACGG GGTGTCCTGC ACACCCACAG TTGAATATCC ATGTGGAAAA |
| | ATGAGAGACG ACCGTCTGCC CCACAGGACG TGTGGGTGTC AACTTATAGG TACACCTTTT |
| 541 | ATACCTATTC TAGAAAAAAG AAATGCCAGC AAACCCCAAG GCCGAAAGAG GAGGAAGAGG |
| | TATGGATAAG ATCTTTTTTC TTTACGGTCG TTTGGGGTTC CGGCTTTCTC CTCCTTCTCC |
| 601 | GGTGGCGGCG GATCAGGTGG GGGTGGATCA GGCGGTGGAG GTTCCCTGCA GGACTCAGAA |
| | CCACCGCCGC CTAGTCCACC CCCACCTAGT CCGCCACCTC CAAGGGACGT CCTGAGTCTT |
| 661 | GTCAATCAAG AAGCTAAGCC AGAGGTCAAG CCAGAAGTCA AGCCTGAGAC TCACATCAAT |
| | CAGTTAGTTC TTCGATTCGG TCTCCAGTTC GGTCTTCAGT TCGGACTCTG AGTGTAGTTA |
| 721 | TTAAAGGTGT CCGATGGATC TTCAGAGATC TTCTTCAAGA TCAAAAAGAC CACTCCTTTA |
| | AATTTCCACA GGCTACCTAG AAGTCTCTAG AAGAAGTTCT AGTTTTTCTG GTGAGGAAAT |
| 781 | AGAAGGCTGA TGGAAGCGTT CGCTAAAAGA CAGGGTAAGG AAATGGACTC CTTAAGATTC |
| | TCTTCCGACT ACCTTCGCAA GCGATTTTCT GTCCCATTCC TTTACCTGAG GAATTCTAAG |
| 841 | TTGTACGACG GTATTAGAAT TCAAGCTGAT CAGGCCCCTG AAGATTTGGA CATGGAGGAT |
| | AACATGCTGC CATAATCTTA AGTTCGACTA GTCCGGGGAC TTCTAAACCT GTACCTCCTA |
| 901 | AACGATATTA TTGAGGCTCA CCGCGAACAG ATTGGAGGTT GCCCCAAAGG GGAGTGTCCA |
| | TTGCTATAAT AACTCCGAGT GGCGCTTGTC TAACCTCCAA CGGGGTTTCC CCTCACAGGT |
| 961 | TGGCAGGTCC TGTTGTTGGT GAATGGAGCT CAGTTGTGTG GGGGGACCCT GATCAACACC |
| | ACCGTCCAGG ACAACAACCA CTTACCTCGA GTCAACACAC CCCCCTGGGA CTAGTTGTGG |
| 1021 | ATCTGGGTGG TCTCCGCGGC CCACTGTTTC GACAAAATCA AGAACTGGAG GAACCTGATC |
| | TAGACCCACC AGAGGCGCCG GGTGACAAAG CTGTTTTAGT TCTTGACCTC CTTGGACTAG |
| 1081 | GCGGTGCTGG GCGAGCACGA CCTCAGCGAG CACGACGGGG ATGAGCAGAG CCGGCGGGTG |
| | CGCCACGACC CGCTCGTGCT GGAGTCGCTC GTGCTGCCCC TACTCGTCTC GGCCGCCCAC |
| 1141 | GCGCAGGTCA TCATCCCCAG CACGTACGTC CCGGGCACCA CCAACCACGA CATCGCGCTG |
| | CGCGTCCAGT AGTAGGGGTC GTGCATGCAG GGCCCGTGGT GGTTGGTGCT GTAGCGCGAC |
| 1201 | CTCCGCCTGC ACCAGCCCGT GGTCCTCACT GACCATGTGG TGCCCCTCTG CCTGCCCGAA |
| | GAGGCGGACG TGGTCGGGCA CCAGGAGTGA CTGGTACACC ACGGGGAGAC GGACGGGCTT |
| 1261 | CGGACGTTCT CTGAGAGGAC GCTGGCCTTC GTGCGCTTCT CATTGGTCAG CGGCTGGGGC |
| | GCCTGCAAGA GACTCTCCTG CGACCGGAAG CACGCGAAGA GTAACCAGTC GCCGACCCCG |
| 1321 | CAGCTGCTGG ACCGTGGCGC CACGGCCCTG GAGCTCATGG TCCTCAACGT GCCCCGGCTG |
| | GTCGACGACC TGGCACCGCG GTGCCGGGAC CTCGAGTACC AGGAGTTGCA CGGGGCCGAC |
| 1381 | ATGACCCAGG ACTGCCTGCA GCAGTCACGG AAGGTGGGAG ACTCCCCAAA TATCACGGAG |
| | TACTGGGTCC TGACGGACGT CGTCAGTGCC TTCCACCCTC TGAGGGGTTT ATAGTGCCTC |
| 1441 | TACATGTTCT GTGCCGGCTA CTCGGATGGC AGCAAGGACT CCTGCAAGGG GGACAGTGGA |
| | ATGTACAAGA CACGGCCGAT GAGCCTACCG TCGTTCCTGA GGACGTTCCC CCTGTCACCT |
| 1501 | GGCCCACATG CCACCCACTA CCGGGGCACG TGGTACCTGA CGGGCATCGT CAGCTGGGGC |
| | CCGGGTGTAC GGTGGGTGAT GGCCCCGTGC ACCATGGACT GCCCGTAGCA GTCGACCCCG |
| 1561 | CAGGGCTGCG CAACCGTGGG CCACTTTGGG GTGTACACCA GGGTCTCCCA GTACATCGAG |
| | GTCCCGACGC GTTGGCACCC GGTGAAACCC CACATGTGGT CCCAGAGGGT CATGTAGCTC |
| 1621 | TGGCTGCAAA AGCTCATGCG CTCAGAGCCA CGCCCAGGAG TCCTCCTGCG AGCCCCATTT |
| | ACCGACGTTT TCGAGTACGC GAGTCTCGGT GCGGGTCCTC AGGAGGACGC TCGGGGTAAA |
| 1681 | CCCGGTGGCG GTGGCTCCGG CGGAGGTGGG TCCGGTGGCG GCGGATCAGG TGGGGGTGGA |
| | GGGCCACCGC CACCGAGGCC GCCTCCACCC AGGCCACCGC CGCCTAGTCC ACCCCCACCT |
| 1741 | TCAGGCGGTG GAGGTTCCGG TGGCGGGGGA TCCGACAAAA CTCACACATG CCCACCGTGC |
| | AGTCCGCCAC CTCCAAGGCC ACCGCCCCCT AGGCTGTTTT GAGTGTGTAC GGGTGGCACG |
| 1801 | CCAGCTCCGG AACTCCTGGG CGGACCGTCA GTCTTCCTCT TCCCCCCAAA ACCCAAGGAC |
| | GGTCGAGGCC TTGAGGACCC GCCTGGCAGT CAGAAGGAGA AGGGGGGTTT TGGGTTCCTG |
| 1861 | ACCCTCATGA TCTCCCGGAC CCCTGAGGTC ACATGCGTGG TGGTGGACGT GAGCCACGAA |
| | TGGGAGTACT AGAGGGCCTG GGGACTCCAG TGTACGCACC ACCACCTGCA CTCGGTGCTT |
| 1921 | GACCCTGAGG TCAAGTTCAA CTGGTACGTG GACGGCGTGG AGGTGCATAA TGCCAAGACA |
| | CTGGGACTCC AGTTCAAGTT GACCATGCAC CTGCCGCACC TCCACGTATT ACGGTTCTGT |
| 1981 | AAGCCGCGGG AGGAGCAGTA CAACAGCACG TACCGTGTGG TCAGCGTCCT CACCGTCCTG |
| | TTCGGCGCCC TCCTCGTCAT GTTGTCGTGC ATGGCACACC AGTCGCAGGA GTGGCAGGAC |
| 2041 | CACCAGGACT GGCTGAATGG CAAGGAGTAC AAGTGCAAGG TCTCCAACAA AGCCCTCCCA |
| | GTGGTCCTGA CCGACTTACC GTTCCTCATG TTCACGTTCC AGAGGTTGTT TCGGGAGGGT |
| 2101 | GCCCCCATCG AGAAAACCAT CTCCAAAGCC AAAGGGCAGC CCCGAGAACC ACAGGTGTAC |
| | CGGGGGTAGC TCTTTTGGTA GAGGTTTCGG TTTCCCGTCG GGGCTCTTGG TGTCCACATG |
| 2161 | ACCCTGCCCC CATCCCGGGA TGAGCTGACC AAGAACCAGG TCAGCCTGAC CTGCCTGGTC |
| | TGGGACGGGG GTAGGGCCCT ACTCGACTGG TTCTTGGTCC AGTCGGACTG GACGGACCAG |
| 2221 | AAAGGCTTCT ATCCCAGCGA CATCGCCGTG GAGTGGGAGA GCAATGGGCA GCCGGAGAAC |
| | TTTCCGAAGA TAGGGTCGCT GTAGCGGCAC CTCACCCTCT CGTTACCCGT CGGCCTCTTG |
| 2281 | AACTACAAGA CCACGCCTCC CGTGTTGGAC TCCGACGGCT CCTTCTTCCT CTACAGCAAG |
| | TTGATGTTCT GGTGCGGAGG GCACAACCTG AGGCTGCCGA GGAAGAAGGA GATGTCGTTC |
| 2341 | CTCACCGTGG ACAAGAGCAG GTGGCAGCAG GGGAACGTCT TCTCATGCTC CGTGATGCAT |
| | GAGTGGCACC TGTTCTCGTC CACCGTCGTC CCCTTGCAGA AGAGTACGAG GCACTACGTA |
| 2401 | GAGGCTCTGC ACAACCACTA CACGCAGAAG AGCCTCTCCC TGTCTCCGGG TAAAGGTGGC |
| | CTCCGAGACG TGTTGGTGAT GTGCGTCTTC TCGGAGAGGG ACAGAGGCCC ATTTCCACCG |
| 2461 | GGCGGATCAG GTGGGGGTGG ATCAGGCGGT GGAGGTTCCG GTGGCGGGGG ATCAGACAAA |
| | CCGCCTAGTC CACCCCCACC TAGTCCGCCA CCTCCAAGGC CACCGCCCCC TAGTCTGTTT |
| 2521 | ACTCACACAT GCCCACCGTG CCCAGCACCT GAACTCCTGG GAGGACCGTC AGTCTTCCTC |
| | TGAGTGTGTA CGGGTGGCAC GGGTCGTGGA CTTGAGGACC CTCCTGGCAG TCAGAAGGAG |
| 2581 | TTCCCCCCAA AACCCAAGGA CACCCTCATG ATCTCCCGGA CCCCTGAGGT CACATGCGTG |
| | AAGGGGGGTT TTGGGTTCCT GTGGGAGTAC TAGAGGGCCT GGGGACTCCA GTGTACGCAC |
| 2641 | GTGGTGGACG TGAGCCACGA AGACCCTGAG GTCAAGTTCA ACTGGTACGT GGACGGCGTG |
| | CACCACCTGC ACTCGGTGCT TCTGGGACTC CAGTTCAAGT TGACCATGCA CCTGCCGCAC |
| 2701 | GAGGTGCATA ATGCCAAGAC AAAGCCGCGG GAGGAGCAGT ACAACAGCAC GTACCGTGTG |
| | CTCCACGTAT TACGGTTCTG TTTCGGCGCC CTCCTCGTCA TGTTGTCGTG CATGGCACAC |
| 2761 | GTCAGCGTCC TCACCGTCCT GCACCAGGAC TGGCTGAATG GCAAGGAGTA CAAGTGCAAG |
| | CAGTCGCAGG AGTGGCAGGA CGTGGTCCTG ACCGACTTAC CGTTCCTCAT GTTCACGTTC |
| 2821 | GTCTCCAACA AAGCCCTCCC AGCCCCCATC GAGAAAACCA TCTCCAAAGC CAAAGGGCAG |
| | CAGAGGTTGT TTCGGGAGGG TCGGGGGTAG CTCTTTTGGT AGAGGTTTCG GTTTCCCGTC |
| 2881 | CCCCGAGAAC CACAGGTGTA CACCCTGCCC CCATCCCGCG ATGAGCTGAC CAAGAACCAG |
| | GGGGCTCTTG GTGTCCACAT GTGGGACGGG GGTAGGGCGC TACTCGACTG GTTCTTGGTC |
| 2941 | GTCAGCCTGA CCTGCCTGGT CAAAGGCTTC TATCCCAGCG ACATCGCCGT GGAGTGGGAG |
| | CAGTCGGACT GGACGGACCA GTTTCCGAAG ATAGGGTCGC TGTAGCGGCA CCTCACCCTC |
| 3001 | AGCAATGGGC AGCCGGAGAA CAACTACAAG ACCACGCCTC CCGTGTTGGA CTCCGACGGC |
| | TCGTTACCCG TCGGCCTCTT GTTGATGTTC TGGTGCGGAG GGCACAACCT GAGGCTGCCG |
| 3061 | TCCTTCTTCC TCTACAGCAA GCTCACCGTG GACAAGAGCA GGTGGCAGCA GGGGAACGTC |
| | AGGAAGAAGG AGATGTCGTT CGAGTGGCAC CTGTTCTCGT CCACCGTCGT CCCCTTGCAG |
| 3121 | TTCTCATGCT CCGTGATGCA TGAGGCTCTG CACAACCACT ACACGCAGAA GAGCCTCTCC |
| | AAGAGTACGA GGCACTACGT ACTCCGAGAC GTGTTGGTGA TGTGCGTCTT CTCGGAGAGG |
| 3181 | CTGTCTCCGG GTAAATGAGA ATTC |
| | GACAGAGGCC CATTTACTCT TAAG |

TABLE 3
FVII-186 amino acid sequence

| | |
|---|---|
| 1 | 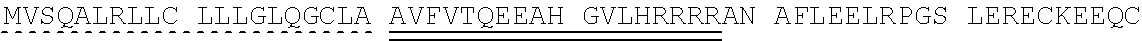 |
| 61 | SFEEAREIFK DAERTKLFWI SYSDGDQCAS SPCQNGGSCK DQLQSYICFC LPAFEGRNCE |
| 121 | THKDDQLICV NENGGCEQYC SDHTGTKRSC RCHEGYSLLA DGVSCTPTVE YPCGKIPILE |
| 181 | 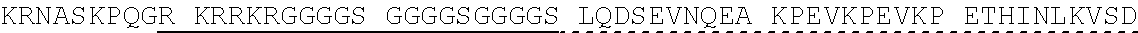 |
| 241 | 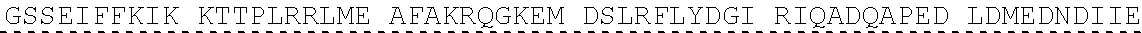 |
| 301 | 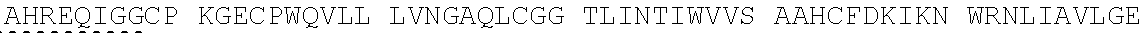 |
| 361 | HDLSEHDGDE QSRRVAQVII PSTYVPGTTN HDIALLRLHQ PVVLTDHVVP LCLPERTFSE |
| 421 | RTLAFVRFSL VSGWGQLLDR GATALELMVL NVPRLMTQDC LQQSRKVGDS PNITEYMFCA |
| 481 | GYSDGSKDSC KGDSGGPHAT HYRGTWYLTG IVSWGQGCAT VGHFGVYTRV SQYIEWLQKL |
| 541 | MRSEPRPGVL LRAPFPGGGG SGGGGSGGGG SGGGGSGGGG SGGGGSDKTH TCPPCPAPEL |
| 601 | LGGPSVFLFP PKPKDTLMIS RTPEVTCVVV DVSHEDPEVK FNWYVDGVEV HNAKTKPREE |
| 661 | QYNSTYRVVS VLTVLHQDWL NGKEYKCKVS NKALPAPIEK TISKAKGQPR EPQVYTLPPS |
| 721 | RDELTKNQVS LTCLVKGFYP SDIAVEWESN GQPENNYKTT PPVLDSDGSF FLYSKLTVDK |
| 781 | SRWQQGNVFS CSVMHEALHN HYTQKSLSLS PGKGGGGSGG GGSGGGGSGG GGSDKTHTCP |
| 841 | PCPAPELLGG PSVFLFPPKP KDTLMISRTP EVTCVVVDVS HEDPEVKFNW YVDGVEVHNA |
| 901 | KTKPREEQYN STYRVVSVLT VLHQDWLNGK EYKCKVSNKA LPAPIEKTIS KAKGQPREPQ |
| 961 | VYTLPPSRDE LTKNQVSLTC LVKGFYPSDI AVEWESNGQP ENNYKTTPPV LDSDGSFFLY |
| 1021 | SKLTVDKSRW QQGNVFSCSV MHEALHNHYT QKSLSLSPGK* |

Signal sequence is shown in dotted underline, propeptide is double underlined, linker with proprotein convertase processing sites connecting the FVII light chain to SUMO is underlined, SUMO sequence is in dashed underline, linker region connecting FVII to Fc region is in italic, and linker connecting the two Fc fragments is shown in bold (SEQ ID NO: 66)

TABLE 4
DNA sequence of FX-011 (SEQ ID NO: 67)

| | |
|---|---|
| 1 | AAGCTTATGG GTCGTCCACT GCACCTCGTC CTGCTCAGTG CCTCCCTGGC TGGCCTCCTG |
| | TTCGAATACC CAGCAGGTGA CGTGGAGCAG GACGAGTCAC GGAGGGACCG ACCGGAGGAC |
| 61 | CTGCTCGGGG AAAGTCTGTT CATCCGCAGG GAGCAGGCCA ACAACATCCT GGCGAGGGTC |
| | GACGAGCCCC TTTCAGACAA GTAGGCGTCC CTCGTCCGGT TGTTGTAGGA CCGCTCCCAG |
| 121 | AGGAGGGCCA ATTCCTTTCT TGAAGAGATG AAGAAAGGAC ACCTCGAAAG AGAGTGCATG |
| | TCCTCCCGGT TAAGGAAAGA ACTTCTCTAC TTCTTTCCTG TGGAGCTTTC TCTCACGTAC |
| 181 | GAAGAGACCT GCTCATACGA AGAGGCCCGC GAGGTCTTTG AGGACAGCGA CAAGACGAAT |
| | CTTCTCTGGA CGAGTATGCT TCTCCGGGCG CTCCAGAAAC TCCTGTCGCT GTTCTGCTTA |
| 241 | GAATTCTGGA ATAAATACAA AGATGGCGAC CAGTGTGAGA CCAGTCCTTG CCAGAACCAG |
| | CTTAAGACCT TATTTATGTT TCTACCGCTG GTCACACTCT GGTCAGGAAC GGTCTTGGTC |
| 301 | GGCAAATGTA AAGACGGCCT CGGGGAATAC ACCTGCACCT GTTTAGAAGG ATTCGAAGGC |
| | CCGTTTACAT TTCTGCCGGA GCCCCTTATG TGGACGTGGA CAAATCTTCC TAAGCTTCCG |
| 361 | AAAAACTGTG AATTATTCAC ACGGAAGCTC TGCAGCCTGG ACAACGGGGA CTGTGACCAG |
| | TTTTTGACAC TTAATAAGTG TGCCTTCGAG ACGTCGGACC TGTTGCCCCT GACACTGGTC |
| 421 | TTCTGCCACG AGGAACAGAA CTCTGTGGTG TGCTCCTGCG CCCGCGGGTA CACCCTGGCT |
| | AAGACGGTGC TCCTTGTCTT GAGACACCAC ACGAGGACGC GGGCGCCCAT GTGGGACCGA |
| 481 | GACAACGGCA AGGCCTGCAT TCCCACAGGG CCCTACCCCT GTGGGAAACA GACCCTGGAA |
| | CTGTTGCCGT TCCGGACGTA AGGGTGTCCC GGGATGGGGA CACCCTTTGT CTGGGACCTT |
| 541 | CGCAGGAAGA GGTCAGTGGC CCAGGCCACC AGCAGCAGCG GGGAGGCCCC TGACAGCATC |
| | GCGTCCTTCT CCAGTCACCG GGTCCGGTGG TCGTCGTCGC CCCTCCGGGG ACTGTCGTAG |
| 601 | ACATGGAAGC CATATGATGC AGCCGACCTG GACCCCACCG AGAACCCCTT CGACCTGCTT |
| | TGTACCTTCG GTATACTACG TCGGCTGGAC CTGGGGTGGC TCTTGGGGAA GCTGGACGAA |
| 661 | GACTTCAACC AGACGCAGCC TGAGAGGGGC GACAACAACG GTGGCGGCGG ATCAGGTGGG |
| | CTGAAGTTGG TCTGCGTCGG ACTCTCCCCG CTGTTGTTGC CACCGCCGCC TAGTCCACCC |
| 721 | GGTGGATCAG GCGGTGGAGG TTCCGGTGGC GGGGGATCCA GGAAGAGGAG GAAGAGGTGC |
| | CCACCTAGTC CGCCACCTCC AAGGCCACCG CCCCCTAGGT CCTTCTCCTC CTTCTCCACG |
| 781 | AAGGACGGGG AGTGTCCCTG GCAGGCCCTG CTCATCAATG AGGAAAACGA GGGTTTTTGT |
| | TTCCTGCCCC TCACAGGGAC CGTCCGGGAC GAGTAGTTAC TCCTTTTGCT CCCAAAAACA |
| 841 | GGAGGTACCA TTCTGAGCGA GTTCTACATC CTAACGGCAG CCCACTGTCT CTACCAAGCC |
| | CCTCCATGGT AAGACTCGCT CAAGATGTAG GATTGCCGTC GGGTGACAGA GATGGTTCGG |
| 901 | AAGAGATTCA AGGTGAGGGT AGGGGACCGG AACACGGAGC AGGAGGAGGG CGGTGAGGCG |
| | TTCTCTAAGT TCCACTCCCA TCCCCTGGCC TTGTGCCTCG TCCTCCTCCC GCCACTCCGC |
| 961 | GTGCACGAGG TGGAGGTGGT CATCAAGCAC AACCGGTTCA CAAAGGAGAC CTATGACTTC |
| | CACGTGCTCC ACCTCCACCA GTAGTTCGTG TTGGCCAAGT GTTTCCTCTG GATACTGAAG |
| 1021 | GACATCGCCG TGCTCCGGCT CAAGACCCCC ATCACCTTCC GCATGAACGT GGCGCCTGCC |
| | CTGTAGCGGC ACGAGGCCGA GTTCTGGGGG TAGTGGAAGG CGTACTTGCA CCGCGGACGG |
| 1081 | TGCCTCCCCG AGCGTGACTG GGCCGAGTCC ACGCTGATGA CGCAGAAGAC GGGGATTGTG |
| | ACGGAGGGGC TCGCACTGAC CCGGCTCAGG TGCGACTACT GCGTCTTCTG CCCCTAACAC |
| 1141 | AGCGGCTTCG GGCGCACCCA CGAGAAGGGC CGGCAGTCCA CCAGGCTCAA GATGCTGGAG |
| | TCGCCGAAGC CCGCGTGGGT GCTCTTCCCG GCCGTCAGGT GGTCCGAGTT CTACGACCTC |
| 1201 | GTGCCCTACG TGGACCGCAA CAGCTGCAAG CTGTCCAGCA GCTTCATCAT CACCCAGAAC |
| | CACGGGATGC ACCTGGCGTT GTCGACGTTC GACAGGTCGT CGAAGTAGTA GTGGGTCTTG |
| 1261 | ATGTTCTGTG CCGGCTACGA CACCAAGCAG GAGGATGCCT GCCAGGGGGA CAGCGGGGGC |
| | TACAAGACAC GGCCGATGCT GTGGTTCGTC CTCCTACGGA CGGTCCCCCT GTCGCCCCCG |
| 1321 | CCGCACGTCA CCCGCTTCAA GGACACCTAC TTCGTGACAG GCATCGTCAG CTGGGGAGAG |
| | GGCGTGCAGT GGGCGAAGTT CCTGTGGATG AAGCACTGTC CGTAGCAGTC GACCCCTCTC |
| 1381 | GGCTGTGCCC GTAAGGGGAA GTACGGGATC TACACCAAGG TCACCGCCTT CCTCAAGTGG |
| | CCGACACGGG CATTCCCCTT CATGCCCTAG ATGTGGTTCC AGTGGCGGAA GGAGTTCACC |
| 1441 | ATCGACAGGT CCATGAAAAC CAGGGGCTTG CCCAAGGCCA AGAGCCATGC CCCGGAGGTC |
| | TAGCTGTCCA GGTACTTTTG GTCCCCGAAC GGGTTCCGGT TCTCGGTACG GGGCCTCCAG |
| 1501 | ATAACGTCCT CTCCATTAAA AGAAGACCAA GTAGATCCGC GGCTCATTGA TGGTAAGGAC |
| | TATTGCAGGA GAGGTAATTT TCTTCTGGTT CATCTAGGCG CCGAGTAACT ACCATTCCTG |
| 1561 | AAAACTCACA CATGCCCACC GTGCCCAGCT CCGGAACTCC TGGGAGGACC GTCAGTCTTC |
| | TTTTGAGTGT GTACGGGTGG CACGGGTCGA GGCCTTGAGG ACCCTCCTGG CAGTCAGAAG |
| 1621 | CTCTTCCCCC CAAAACCCAA GGACACCCTC ATGATCTCCC GGACCCCTGA GGTCACATGC |
| | GAGAAGGGGG GTTTTGGGTT CCTGTGGGAG TACTAGAGGG CCTGGGGACT CCAGTGTACG |
| 1681 | GTGGTGGTGG ACGTGAGCCA CGAAGACCCT GAGGTCAAGT TCAACTGGTA CGTGGACGGC |
| | CACCACCACC TGCACTCGGT GCTTCTGGGA CTCCAGTTCA AGTTGACCAT GCACCTGCCG |
| 1741 | GTGGAGGTGC ATAATGCCAA GACAAAGCCG CGGGAGGAGC AGTACAACAG CACGTACCGT |
| | CACCTCCACG TATTACGGTT CTGTTTCGGC GCCCTCCTCG TCATGTTGTC GTGCATGGCA |
| 1801 | GTGGTCAGCG TCCTCACCGT CCTGCACCAG GACTGGCTGA ATGGCAAGGA GTACAAGTGC |
| | CACCAGTCGC AGGAGTGGCA GGACGTGGTC CTGACCGACT TACCGTTCCT CATGTTCACG |
| 1861 | AAGGTCTCCA ACAAAGCCCT CCCAGCCCCC ATCGAGAAAA CCATCTCCAA AGCCAAAGGG |
| | TTCCAGAGGT TGTTTCGGGA GGGTCGGGGG TAGCTCTTTT GGTAGAGGTT TCGGTTTCCC |
| 1921 | CAGCCCCGAG AACCACAGGT GTACACCCTG CCCCCATCCC GGGATGAGCT GACCAAGAAC |
| | GTCGGGGCTC TTGGTGTCCA CATGTGGGAC GGGGGTAGGG CCCTACTCGA CTGGTTCTTG |
| 1981 | CAGGTCAGCC TGACCTGCCT GGTCAAAGGC TTCTATCCCA GCGACATCGC CGTGGAGTGG |
| | GTCCAGTCGG ACTGGACGGA CCAGTTTCCG AAGATAGGGT CGCTGTAGCG GCACCTCACC |
| 2041 | GAGAGCAATG GGCAGCCGGA GAACAACTAC AAGACCACGC CTCCCGTGTT GGACTCCGAC |
| | CTCTCGTTAC CCGTCGGCCT CTTGTTGATG TTCTGGTGCG GAGGGCACAA CCTGAGGCTG |
| 2101 | GGCTCCTTCT TCCTCTACAG CAAGCTCACC GTCGACAAGA GCAGGTGGCA GCAGGGGAAC |
| | CCGAGGAAGA AGGAGATGTC GTTCGAGTGG CAGCTGTTCT CGTCCACCGT CGTCCCCTTG |
| 2161 | GTCTTCTCAT GCTCCGTGAT GCATGAGGCT CTGCACAACC ACTACACGCA GAAGAGCCTC |
| | CAGAAGAGTA CGAGGCACTA CGTACTCCGA GACGTGTTGG TGATGTGCGT CTTCTCGGAG |
| 2221 | TCCCTGTCTC CGGGTAAACG GCGCCGCCGG AGCGGTGGCG GCGGATCAGG TGGGGGTGGA |
| | AGGGACAGAG GCCCATTTGC CGCGGCGGCC TCGCCACCGC CGCCTAGTCC ACCCCCACCT |
| 2281 | TCAGGCGGTG GAGGTTCCGG TGGCGGGGGA TCCAGGAAGA GGAGGAAGAG GGACAAAACT |
| | AGTCCGCCAC CTCCAAGGCC ACCGCCCCCT AGGTCCTTCT CCTCCTTCTC CCTGTTTTGA |
| 2341 | CACACATGCC CACCGTGCCC AGCACCGGAA CTCCTGGGCG GACCGTCAGT CTTCCTCTTC |
| | GTGTGTACGG GTGGCACGGG TCGTGGCCTT GAGGACCCGC CTGGCAGTCA GAAGGAGAAG |
| 2401 | CCCCCAAAAC CCAAGGACAC CCTCATGATC TCCCGGACCC CTGAGGTCAC ATGCGTGGTG |
| | GGGGGTTTTG GGTTCCTGTG GGAGTACTAG AGGGCCTGGG GACTCCAGTG TACGCACCAC |
| 2461 | GTGGACGTGA GCCACGAAGA CCCTGAGGTC AAGTTCAACT GGTACGTGGA CGGCGTGGAG |
| | CACCTGCACT CGGTGCTTCT GGGACTCCAG TTCAAGTTGA CCATGCACCT GCCGCACCTC |
| 2521 | GTGCATAATG CCAAGACAAA GCCGCGGGAG GAGCAGTACA ACAGCACGTA CCGTGTGGTC |
| | CACGTATTAC GGTTCTGTTT CGGCGCCCTC CTCGTCATGT TGTCGTGCAT GGCACACCAG |
| 2581 | AGCGTCCTCA CCGTCCTGCA CCAGGACTGG CTGAATGGCA AGGAGTACAA GTGCAAGGTC |
| | TCGCAGGAGT GGCAGGACGT GGTCCTGACC GACTTACCGT TCCTCATGTT CACGTTCCAG |
| 2641 | TCCAACAAAG CCCTCCCAGC CCCCATCGAG AAAACCATCT CCAAAGCCAA AGGGCAGCCC |
| | AGGTTGTTTC GGGAGGGTCG GGGGTAGCTC TTTTGGTAGA GGTTTCGGTT TCCCGTCGGG |
| 2701 | CGAGAACCAC AGGTGTACAC CCTGCCCCCA TCCCGGGATG AGCTGACCAA GAACCAGGTC |
| | GCTCTTGGTG TCCACATGTG GGACGGGGGT AGGGCCCTAC TCGACTGGTT CTTGGTCCAG |
| 2761 | AGCCTGACCT GCCTGGTCAA AGGCTTCTAT CCCAGCGACA TCGCCGTGGA GTGGGAGAGC |
| | TCGGACTGGA CGGACCAGTT TCCGAAGATA GGGTCGCTGT AGCGGCACCT CACCCTCTCG |
| 2821 | AATGGGCAGC CGGAGAACAA CTACAAGACC ACGCCTCCCG TGTTGGACTC CGACGGCTCC |
| | TTACCCGTCG GCCTCTTGTT GATGTTCTGG TGCGGAGGGC ACAACCTGAG GCTGCCGAGG |
| 2881 | TTCTTCCTCT ACAGCAAGCT CACCGTGGAC AAGAGCAGGT GGCAGCAGGG GAACGTCTTC |
| | AAGAAGGAGA TGTCGTTCGA GTGGCACCTG TTCTCGTCCA CCGTCGTCCC CTTGCAGAAG |
| 2941 | TCATGCTCCG TGATGCATGA GGCTCTGCAC AACCACTACA CGCAGAAGAG CCTCTCCCTG |
| | AGTACGAGGC ACTACGTACT CCGAGACGTG TTGGTGATGT GCGTCTTCTC GGAGAGGGAC |
| 3001 | TCTCCGGGTA AATGAGCGGC CGC |
| | AGAGGCCCAT TTACTCGCCG GCG |

TABLE 5
FX-011 amino acid sequence.

| | |
|---|---|
| 1 | 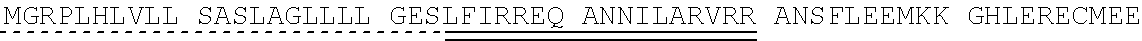 |
| 61 | TCSYEEAREV FEDSDKTNEF WNKYKDGDQC ETSPCQNQGK CKDGLGEYTC TCLEGFEGKN |
| 121 | CELFTRKLCS LDNGDCDQFC HEEQNSVVCS CARGYTLADN GKACIPTGPY PCGKQTLERR |
| 181 | KRSVAQATSS SGEAPDSITW KPYDAADLDP TENPFDLLDF NQTQPERGDN NGGGGSGGGG |
| 241 | SGGGGSGGGG SRKRRKRCKD GECPWQALLI NEENEGFCGG TILSEFYILT AAHCLYQAKR |
| 301 | FKVRVGDRNT EQEEGGEAVH EVEVVIKHNR FTKETYDFDI AVLRLKTPIT FRMNVAPACL |
| 361 | PERDWAESTL MTQKTGIVSG FGRTHEKGRQ STRLKMLEVP YVDRNSCKLS SSFIITQNMF |
| 421 | CAGYDTKQED ACQGDSGGPH VTRFKDTYFV TGIVSWGEGC ARKGKYGIYT KVTAFLKWID |
| 481 | RSMKTRGLPK AKSHAPEVIT SSPLKEDQVD PRLIDGKDKT HTCPPCPAPE LLGGPSVFLF |
| 541 | PPKPKDTLMI SRTPEVTCVV VDVSHEDPEV KFNWYVDGVE VHNAKTKPRE EQYNSTYRVV |
| 601 | SVLTVLHQDW LNGKEYKCKV SNKALPAPIE KTISKAKGQP REPQVYTLPP SRDELTKNQV |
| 661 | SLTCLVKGFY PSDIAVEWES NGQPENNYKT TPPVLDSDGS FFLYSKLTVD KSRWQQGNVF |
| 721 | SCSVMHEALH NHYTQKSLSL SPGKRRRRSG GGGSGGGGSG GGGSGGGGSR KRRKRDKTHT |
| 781 | CPPCPAPELL GGPSVFLFPP KPKDTLMISR TPEVTCVVVD VSHEDPEVKF NWYVDGVEVH |
| 841 | NAKTKPREEQ YNSTYRVVSV LTVLHQDWLN GKEYKCKVSN KALPAPIEKT ISKAKGQPRE |
| 901 | PQVYTLPPSR DELTKNQVSL TCLVKGFYPS DIAVEWESNG QPENNYKTTP PVLDSDGSFF |
| 961 | LYSKLTVDKS RWQQGNVFSC SVMHEALHNH YTQKSLSLSP GK* |

Signal sequence is shown in dotted underline, propeptide is double underlined, linker region connecting FX activation peptide to FX heavy chain is underlined, and linker with proprotein convertase processing sites connecting the two Fc fragments is shown in bold (SEQ ID NO: 68)

TABLE 6
DNA sequence of FX-012 (SEQ ID NO: 69)

| | |
|---|---|
| 1 | AAGCTTATGG GTCGTCCACT GCACCTCGTC CTGCTCAGTG CCTCCCTGGC TGGCCTCCTG |
| | TTCGAATACC CAGCAGGTGA CGTGGAGCAG GACGAGTCAC GGAGGGACCG ACCGGAGGAC |
| 61 | CTGCTCGGGG AAAGTCTGTT CATCCGCAGG GAGCAGGCCA ACAACATCCT GGCGAGGGTC |
| | GACGAGCCCC TTTCAGACAA GTAGGCGTCC CTCGTCCGGT TGTTGTAGGA CCGCTCCCAG |
| 121 | AGGAGGGCCA ATTCCTTTCT TGAAGAGATG AAGAAAGGAC ACCTCGAAAG AGAGTGCATG |
| | TCCTCCCGGT TAAGGAAAGA ACTTCTCTAC TTCTTTCCTG TGGAGCTTTC TCTCACGTAC |
| 181 | GAAGAGACCT GCTCATACGA AGAGGCCCGC GAGGTCTTTG AGGACAGCGA CAAGACGAAT |
| | CTTCTCTGGA CGAGTATGCT TCTCCGGGCG CTCCAGAAAC TCCTGTCGCT GTTCTGCTTA |
| 241 | GAATTCTGGA ATAAATACAA AGATGGCGAC CAGTGTGAGA CCAGTCCTTG CCAGAACCAG |
| | CTTAAGACCT TATTTATGTT TCTACCGCTG GTCACACTCT GGTCAGGAAC GGTCTTGGTC |
| 301 | GGCAAATGTA AAGACGGCCT CGGGGAATAC ACCTGCACCT GTTTAGAAGG ATTCGAAGGC |
| | CCGTTTACAT TTCTGCCGGA GCCCCTTATG TGGACGTGGA CAAATCTTCC TAAGCTTCCG |
| 361 | AAAAACTGTG AATTATTCAC ACGGAAGCTC TGCAGCCTGG ACAACGGGGA CTGTGACCAG |
| | TTTTTGACAC TTAATAAGTG TGCCTTCGAG ACGTCGGACC TGTTGCCCCT GACACTGGTC |
| 421 | TTCTGCCACG AGGAACAGAA CTCTGTGGTG TGCTCCTGCG CCCGCGGGTA CACCCTGGCT |
| | AAGACGGTGC TCCTTGTCTT GAGACACCAC ACGAGGACGC GGGCGCCCAT GTGGGACCGA |
| 481 | GACAACGGCA AGGCCTGCAT TCCCACAGGG CCCTACCCCT GTGGGAAACA GACCCTGGAA |
| | CTGTTGCCGT TCCGGACGTA AGGGTGTCCC GGGATGGGGA CACCCTTTGT CTGGGACCTT |
| 541 | CGCAGGAAGA GGGGTGGGGG TGGATCAGGC GGTGGAGGTT CCGGTGGCGG GGGATCCTCC |
| | GCGTCCTTCT CCCCACCCCC ACCTAGTCCG CCACCTCCAA GGCCACCGCC CCCTAGGAGG |
| 601 | CTGCAGGACT CAGAAGTCAA TCAAGAAGCT AAGCCAGAGG TCAAGCCAGA AGTCAAGCCT |
| | GACGTCCTGA GTCTTCAGTT AGTTCTTCGA TTCGGTCTCC AGTTCGGTCT TCAGTTCGGA |
| 661 | GAGACTCACA TCAATTTAAA GGTGTCCGAT GGATCTTCAG AGATCTTCTT CAAGATCAAA |
| | CTCTGAGTGT AGTTAAATTT CCACAGGCTA CCTAGAAGTC TCTAGAAGAA GTTCTAGTTT |
| 721 | AAGACCACTC CTTTAAGAAG GCTGATGGAA GCGTTCGCTA AAAGACAGGG TAAGGAAATG |
| | TTCTGGTGAG GAAATTCTTC CGACTACCTT CGCAAGCGAT TTTCTGTCCC ATTCCTTTAC |
| 781 | GACTCCTTAA GATTCTTGTA CGACGGTATT AGAATTCAAG CTGATCAGGC CCCTGAAGAT |
| | CTGAGGAATT CTAAGAACAT GCTGCCATAA TCTTAAGTTC GACTAGTCCG GGGACTTCTA |
| 841 | TTGGACATGG AGGATAACGA TATTATTGAG GCTCACCGCG AACAGATTGG AGGTTGCAAG |
| | AACCTGTACC TCCTATTGCT ATAATAACTC CGAGTGGCGC TTGTCTAACC TCCAACGTTC |
| 901 | GACGGGGAGT GTCCCTGGCA GGCCCTGCTC ATCAATGAGG AAAACGAGGG TTTTTGTGGA |
| | CTGCCCCTCA CAGGGACCGT CCGGGACGAG TAGTTACTCC TTTTGCTCCC AAAAACACCT |
| 961 | GGTACCATTC TGAGCGAGTT CTACATCCTA ACGGCAGCCC ACTGTCTCTA CCAAGCCAAG |
| | CCATGGTAAG ACTCGCTCAA GATGTAGGAT TGCCGTCGGG TGACAGAGAT GGTTCGGTTC |
| 1021 | AGATTCAAGG TGAGGGTAGG GGACCGGAAC ACGGAGCAGG AGGAGGGCGG TGAGGCGGTG |
| | TCTAAGTTCC ACTCCCATCC CCTGGCCTTG TGCCTCGTCC TCCTCCCGCC ACTCCGCCAC |
| 1081 | CACGAGGTGG AGGTGGTCAT CAAGCACAAC CGGTTCACAA AGGAGACCTA TGACTTCGAC |
| | GTGCTCCACC TCCACCAGTA GTTCGTGTTG GCCAAGTGTT TCCTCTGGAT ACTGAAGCTG |
| 1141 | ATCGCCGTGC TCCGGCTCAA GACCCCCATC ACCTTCCGCA TGAACGTGGC GCCTGCCTGC |
| | TAGCGGCACG AGGCCGAGTT CTGGGGGTAG TGGAAGGCGT ACTTGCACCG CGGACGGACG |
| 1201 | CTCCCCGAGC GTGACTGGGC CGAGTCCACG CTGATGACGC AGAAGACGGG GATTGTGAGC |
| | GAGGGGCTCG CACTGACCCG GCTCAGGTGC GACTACTGCG TCTTCTGCCC CTAACACTCG |
| 1261 | GGCTTCGGGC GCACCCACGA GAAGGGCCGG CAGTCCACCA GGCTCAAGAT GCTGGAGGTG |
| | CCGAAGCCCG CGTGGGTGCT CTTCCCGGCC GTCAGGTGGT CCGAGTTCTA CGACCTCCAC |
| 1321 | CCCTACGTGG ACCGCAACAG CTGCAAGCTG TCCAGCAGCT TCATCATCAC CCAGAACATG |
| | GGGATGCACC TGGCGTTGTC GACGTTCGAC AGGTCGTCGA AGTAGTAGTG GGTCTTGTAC |
| 1381 | TTCTGTGCCG GCTACGACAC CAAGCAGGAG GATGCCTGCC AGGGGGACAG CGGGGGCCCG |
| | AAGACACGGC CGATGCTGTG GTTCGTCCTC CTACGGACGG TCCCCCTGTC GCCCCCGGGC |
| 1441 | CACGTCACCC GCTTCAAGGA CACCTACTTC GTGACAGGCA TCGTCAGCTG GGGAGAGGGC |
| | GTGCAGTGGG CGAAGTTCCT GTGGATGAAG CACTGTCCGT AGCAGTCGAC CCCTCTCCCG |
| 1501 | TGTGCCCGTA AGGGGAAGTA CGGGATCTAC ACCAAGGTCA CCGCCTTCCT CAAGTGGATC |
| | ACACGGGCAT TCCCCTTCAT GCCCTAGATG TGGTTCCAGT GGCGGAAGGA GTTCACCTAG |
| 1561 | GACAGGTCCA TGAAAACCAG GGGCTTGCCC AAGGCCAAGA GCCATGCCCC GGAGGTCATA |
| | CTGTCCAGGT ACTTTTGGTC CCCGAACGGG TTCCGGTTCT CGGTACGGGG CCTCCAGTAT |
| 1621 | ACGTCCTCTC CATTAAAAGA AGACCAAGTA GATCCGCGGC TCATTGATGG TAAGGACAAA |
| | TGCAGGAGAG GTAATTTTCT TCTGGTTCAT CTAGGCGCCG AGTAACTACC ATTCCTGTTT |
| 1681 | ACTCACACAT GCCCACCGTG CCCAGCTCCG GAACTCCTGG GAGGACCGTC AGTCTTCCTC |
| | TGAGTGTGTA CGGGTGGCAC GGGTCGAGGC CTTGAGGACC CTCCTGGCAG TCAGAAGGAG |
| 1741 | TTCCCCCCAA AACCCAAGGA CACCCTCATG ATCTCCCGGA CCCCTGAGGT CACATGCGTG |
| | AAGGGGGGTT TTGGGTTCCT GTGGGAGTAC TAGAGGGCCT GGGGACTCCA GTGTACGCAC |
| 1801 | GTGGTGGACG TGAGCCACGA AGACCCTGAG GTCAAGTTCA ACTGGTACGT GGACGGCGTG |
| | CACCACCTGC ACTCGGTGCT TCTGGGACTC CAGTTCAAGT TGACCATGCA CCTGCCGCAC |
| 1861 | GAGGTGCATA ATGCCAAGAC AAAGCCGCGG GAGGAGCAGT ACAACAGCAC GTACCGTGTG |
| | CTCCACGTAT TACGGTTCTG TTTCGGCGCC CTCCTCGTCA TGTTGTCGTG CATGGCACAC |
| 1921 | GTCAGCGTCC TCACCGTCCT GCACCAGGAC TGGCTGAATG GCAAGGAGTA CAAGTGCAAG |
| | CAGTCGCAGG AGTGGCAGGA CGTGGTCCTG ACCGACTTAC CGTTCCTCAT GTTCACGTTC |
| 1981 | GTCTCCAACA AAGCCCTCCC AGCCCCCATC GAGAAAACCA TCTCCAAAGC CAAAGGGCAG |
| | CAGAGGTTGT TTCGGGAGGG TCGGGGGTAG CTCTTTTGGT AGAGGTTTCG GTTTCCCGTC |
| 2041 | CCCCGAGAAC CACAGGTGTA CACCCTGCCC CCATCCCGGG ATGAGCTGAC CAAGAACCAG |
| | GGGGCTCTTG GTGTCCACAT GTGGGACGGG GGTAGGGCCC TACTCGACTG GTTCTTGGTC |
| 2101 | GTCAGCCTGA CCTGCCTGGT CAAAGGCTTC TATCCCAGCG ACATCGCCGT GGAGTGGGAG |
| | CAGTCGGACT GGACGGACCA GTTTCCGAAG ATAGGGTCGC TGTAGCGGCA CCTCACCCTC |
| 2161 | AGCAATGGGC AGCCGGAGAA CAACTACAAG ACCACGCCTC CCGTGTTGGA CTCCGACGGC |
| | TCGTTACCCG TCGGCCTCTT GTTGATGTTC TGGTGCGGAG GGCACAACCT GAGGCTGCCG |
| 2221 | TCCTTCTTCC TCTACAGCAA GCTCACCGTC GACAAGAGCA GGTGGCAGCA GGGGAACGTC |
| | AGGAAGAAGG AGATGTCGTT CGAGTGGCAG CTGTTCTCGT CCACCGTCGT CCCCTTGCAG |
| 2281 | TTCTCATGCT CCGTGATGCA TGAGGCTCTG CACAACCACT ACACGCAGAA GAGCCTCTCC |
| | AAGAGTACGA GGCACTACGT ACTCCGAGAC GTGTTGGTGA TGTGCGTCTT CTCGGAGAGG |
| 2341 | CTGTCTCCGG GTAAACGGCG CCGCCGGAGC GGTGGCGGCG GATCAGGTGG GGGTGGATCA |
| | GACAGAGGCC CATTTGCCGC GGCGGCCTCG CCACCGCCGC CTAGTCCACC CCCACCTAGT |
| 2401 | GGCGGTGGAG GTTCCGGTGG CGGGGGATCC AGGAAGAGGA GGAAGAGGGA CAAAACTCAC |
| | CCGCCACCTC CAAGGCCACC GCCCCCTAGG TCCTTCTCCT CCTTCTCCCT GTTTTGAGTG |
| 2461 | ACATGCCCAC CGTGCCCAGC ACCGGAACTC CTGGGCGGAC CGTCAGTCTT CCTCTTCCCC |
| | TGTACGGGTG GCACGGGTCG TGGCCTTGAG GACCCGCCTG GCAGTCAGAA GGAGAAGGGG |
| 2521 | CCAAAACCCA AGGACACCCT CATGATCTCC CGGACCCCTG AGGTCACATG CGTGGTGGTG |
| | GGTTTTGGGT TCCTGTGGGA GTACTAGAGG GCCTGGGGAC TCCAGTGTAC GCACCACCAC |
| 2581 | GACGTGAGCC ACGAAGACCC TGAGGTCAAG TTCAACTGGT ACGTGGACGG CGTGGAGGTG |
| | CTGCACTCGG TGCTTCTGGG ACTCCAGTTC AAGTTGACCA TGCACCTGCC GCACCTCCAC |
| 2641 | CATAATGCCA AGACAAAGCC GCGGGAGGAG CAGTACAACA GCACGTACCG TGTGGTCAGC |
| | GTATTACGGT TCTGTTTCGG CGCCCTCCTC GTCATGTTGT CGTGCATGGC ACACCAGTCG |
| 2701 | GTCCTCACCG TCCTGCACCA GGACTGGCTG AATGGCAAGG AGTACAAGTG CAAGGTCTCC |
| | CAGGAGTGGC AGGACGTGGT CCTGACCGAC TTACCGTTCC TCATGTTCAC GTTCCAGAGG |
| 2761 | AACAAAGCCC TCCCAGCCCC CATCGAGAAA ACCATCTCCA AAGCCAAAGG GCAGCCCCGA |
| | TTGTTTCGGG AGGGTCGGGG GTAGCTCTTT TGGTAGAGGT TTCGGTTTCC CGTCGGGGCT |
| 2821 | GAACCACAGG TGTACACCCT GCCCCCATCC CGGGATGAGC TGACCAAGAA CCAGGTCAGC |
| | CTTGGTGTCC ACATGTGGGA CGGGGGTAGG GCCCTACTCG ACTGGTTCTT GGTCCAGTCG |
| 2881 | CTGACCTGCC TGGTCAAAGG CTTCTATCCC AGCGACATCG CCGTGGAGTG GGAGAGCAAT |
| | GACTGGACGG ACCAGTTTCC GAAGATAGGG TCGCTGTAGC GGCACCTCAC CCTCTCGTTA |
| 2941 | GGGCAGCCGG AGAACAACTA CAAGACCACG CCTCCCGTGT TGGACTCCGA CGGCTCCTTC |
| | CCCGTCGGCC TCTTGTTGAT GTTCTGGTGC GGAGGGCACA ACCTGAGGCT GCCGAGGAAG |
| 3001 | TTCCTCTACA GCAAGCTCAC CGTGGACAAG AGCAGGTGGC AGCAGGGGAA CGTCTTCTCA |
| | AAGGAGATGT CGTTCGAGTG GCACCTGTTC TCGTCCACCG TCGTCCCCTT GCAGAAGAGT |
| 3061 | TGCTCCGTGA TGCATGAGGC TCTGCACAAC CACTACACGC AGAAGAGCCT CTCCCTGTCT |
| | ACGAGGCACT ACGTACTCCG AGACGTGTTG GTGATGTGCG TCTTCTCGGA GAGGGACAGA |
| 3121 | CCGGGTAAAT GAGCGGCCGC |
| | GGCCCATTTA CTCGCCGGCG |

TABLE 7
FX-012 amino acid sequence.

| | |
|---|---|
| 1 | 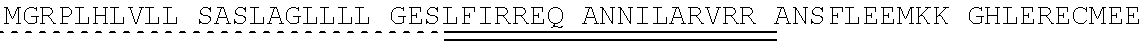 |
| 61 | TCSYEEAREV FEDSDKTNEF WNKYKDGDQC ETSPCQNQGK CKDGLGEYTC TCLEGFEGKN |
| 121 | CELFTRKLCS LDNGDCDQFC HEEQNSVVCS CARGYTLADN GKACIPTGPY PCGKQTLERR |
| 181 | 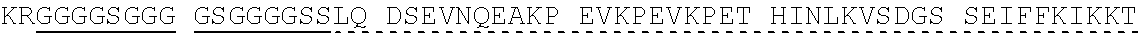 |
| 241 | 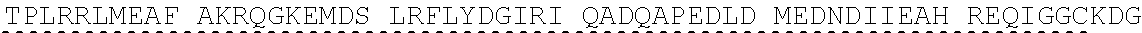 |
| 301 | ECPWQALLIN EENEGFCGGT ILSEFYILTA AHCLYQAKRF KVRVGDRNTE QEEGGEAVHE |
| 361 | VEVVIKHNRF TKETYDFDIA VLRLKTPITF RMNVAPACLP ERDWAESTLM TQKTGIVSGF |
| 421 | GRTHEKGRQS TRLKMLEVPY VDRNSCKLSS SFIITQNMFC AGYDTKQEDA CQGDSGGPHV |
| 481 | TRFKDTYFVT GIVSWGEGCA RKGKYGIYTK VTAFLKWIDR SMKTRGLPKA KSHAPEVITS |
| 541 | SPLKEDQVDP RLIDGKDKTH TCPPCPAPEL LGGPSVFLFP PKPKDTLMIS RTPEVTCVVV |
| 601 | DVSHEDPEVK FNWYVDGVEV HNAKTKPREE QYNSTYRVVS VLTVLHQDWL NGKEYKCKVS |
| 661 | NKALPAPIEK TISKAKGQPR EPQVYTLPPS RDELTKNQVS LTCLVKGFYP SDIAVEWESN |
| 721 | GQPENNYKTT PPVLDSDGSF FLYSKLTVDK SRWQQGNVFS CSVMHEALHN HYTQKSLSLS |
| 781 | PGKRRRRSGG GGSGGGGSGG GGSGGGGSRK RRKRDKTHTC PPCPAPELLG GPSVFLFPPK |
| 841 | PKDTLMISRT PEVTCVVVDV SHEDPEVKFN WYVDGVEVHN AKTKPREEQY NSTYRVVSVL |
| 901 | TVLHQDWLNG KEYKCKVSNK ALPAPIEKTI SKAKGQPREP QVYTLPPSRD ELTKNQVSLT |
| 961 | CLVKGFYPSD IAVEWESNGQ PENNYKTTPP VLDSDGSFFL YSKLTVDKSR WQQGNVFSCS |
| 1021 | VMHEALHNHY TQKSLSLSPG K* |

Signal sequence is shown in dotted underline, propeptide is double underlined, linker region connecting FX light chain to SUMO is underlined, SUMO sequence is in dashed underline, and linker with proprotein convertase processing sites connecting the two Fc fragments is shown in bold (SEQ ID NO: 70)

SEQUENCES
>CTP peptide 1
SEQ ID NO: 51
DPRFQDSSSSKAPPPSLPSPSRLPGPSDTPIL
>CTP peptide 2
SEQ ID NO: 52
SSSSKAPPPSLPSPSRLPGPSDTPILPQ
>PAS peptide 1
SEQ ID NO: 58
ASPAAPAPASPAAPAPSAPA
>PAS peptide 2
SEQ ID NO: 59
AAPASPAPAAPSAPAPAAPS
>PAS peptide 3
SEQ ID NO: 60
APSSPSPSAPSSPSPASPSS
>PAS peptide 4
SEQ ID NO: 61
APSSPSPSAPSSPSPASPS
>PAS peptide 5
SEQ ID NO: 62
SSPSAPSPSSPASPSPSSPA
>PAS peptide 6
SEQ ID NO: 63
AASPAAPSAPPAAASPAAPSAPPA
>PAS peptide 7
SEQ ID NO: 64
ASAAAPAAASAAASAPSAAA
>Albumin Binding Peptide Core Sequence
SEQ ID NO: 53
DICLPRWGCLW
>GFP protein sequence (Genbank ID AAG34521.1)
SEQ ID NO: 71
MSKGEELFTGVVPILVELDGDVNGHKFSVSGEGEGDATYGKLTLKFICTTGKLPVPWPTL
VTTFGYGVQCFARYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHNIEDGSVQLAD
HYQQNTPIGDGPVLLPDNHYLSTQSALSKDPNEKRDHMVLLEFVTAAGITHGMDELYKSR
TSGSPGLQEFDIKLIDTVDLESCN
>Example: Single-chain Human IgG1 Fc. (Fc sequences with
Gly/Ser linker underlined.)
SEQ ID NO: 72
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGKGGGGSGGGGSGGG
GSGGGGSDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
>Mature human albumin protein sequence (derived from NCBI
Ref. Sequence NP_000468):
SEQ ID NO: 73
RGVFRRDAHKSEVAHRFKDLGEENFKALVLIAFAQYLQQCPFEDHVKLVNEVTEFAKTCV
ADESAENCDKSLHTLFGDKLCTVATLRETYGEMADCCAKQEPERNECFLQHKDDNPNLPR
LVRPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVSKL
VTDLTKVHTECCHGDLLECADDRADLAKYICENQDSISSKLKECCEKPLLEKSHCIAEVE
NDEMPADLPSLAADFVESKDVCKNYAEAKDVFLGMFLYEYARRHPDYSVVLLLRLAKTYE
TTLEKCCAAADPHECYAKVFDEFKPLVEEPQNLIKQNCELFEQLGEYKFQNALLVRYTKK
VPQVSTPTLVEVSRNLGKVGSKCCKHPEAKRMPCAEDYLSVVLNQLCVLHEKTPVSDRVT
KCCTESLVNRRPCFSALEVDETYVPKEFNAETFTFHADICTLSEKERQIKKQTALVELVK
HKPKATKEQLKAVMDDFAAFVEKCCKADDKETCFAEEGKKLVAASQAALGL
>Albumin binding peptide 1
SEQ ID NO: 54
RLIEDICLPRWGCLWEDD
>Albumin binding peptide 2
SEQ ID NO: 55
QRLMEDICLPRWGCLWEDDF
>Albumin binding peptide 3
SEQ ID NO: 56
QGLIGDICLPRWGCLWGDSVK
>Albumin binding peptide 4
SEQ ID NO: 74
GEWWEDICLPRWGCLWEEED
>Cysteine-containing peptide
SEQ ID NO: 75
GGGSGCGGGS
>Human LRP1 sequence (signal peptide and transmembrane
segment underlined; NCBI Reference Sequence: CAA32112)
SEQ ID NO: 76
MLTPPLLLLLPLLSALVAAAIDAPKTCSPKQFACRDQITCISKGWRCDGERDCPDGSDEA
PEICPQSKAQRCQPNEHNCLGTELCVPMSRLCNGVQDCMDGSDEGPHCRELQGNCSRLGC
QHHCVPTLDGPTCYCNSSFQLQADGKTCKDFDECSVYGTCSQLCTNTDGSFICGCVEGYL
LQPDNRSCKAKNEPVDRPPVLLIANSQNILATYLSGAQVSTITPTSTRQTTAMDFSYANE
TVCWVHVGDSAAQTQLKCARMPGLKGFVDEHTINISLSLHHVEQMAIDWLTGNFYFVDDI
DDRIFVCNRNGDTCVTLLDLELYNPKGIALDPAMGKVFFTDYGQIPKVERCDMDGQNRTK
LVDSKIVFPHGITLDLVSRLVYWADAYLDYIEVVDYEGKGRQTIIQGILIEHLYGLTVFE
NYLYATNSDNANAQQKTSVIRVNRFNSTEYQVVTRVDKGGALHIYHQRRQPRVRSHACEN
DQYGKPGGCSDICLLANSHKARTCRCRSGFSLGSDGKSCKKPEHELFLVYGKGRPGIIRG
MDMGAKVPDEHMIPIENLMNPRALDFHAETGFIYFADTTSYLIGRQKIDGTERETILKDG
IHNVEGVAVDWMGDNLYWTDDGPKKTISVARLEKAAQTRKTLIEGKMTHPRAIVVDPLNG
WMYWTDWEEDPKDSRRGRLERAWMDGSHRDIFVTSKTVLWPNGLSLDIPAGRLYWVDAFY
DRIETILLNGTDRKIVYEGPELNHAFGLCHHGNYLFWTEYRSGSVYRLERGVGGAPPTVT
LLRSERPPIFEIRMYDAQQQQVGTNKCRVNNGGCSSLCLATPGSRQCACAEDQVLDADGV
TCLANPSYVPPPQCQPGEFACANSRCIQERWKCDGDNDCLDNSDEAPALCHQHTCPSDRF
KCENNRCIPNRWLCDGDNDCGNSEDESNATCSARTCPPNQFSCASGRCIPISWTCDLDDD
CGDRSDESASCAYPTCFPLTQFTCNNGRCININWRCDNDNDCGDNSDEAGCSHSCSSTQF
KCNSGRCIPEHWTCDGDNDCGDYSDETHANCTNQATRPPGGCHTDEFQCRLDGLCIPLRW
RCDGDTDCMDSSDEKSCEGVTHVCDPSVKFGCKDSARCISKAWVCDGDNDCEDNSDEENC
ESLACRPPSHPCANNTSVCLPPDKLCDGNDDCGDGSDEGELCDQCSLNNGGCSHNCSVAP
GEGIVCSCPLGMELGPDNHTCQIQSYCAKHLKCSQKCDQNKFSVKCSCYEGWVLEPDGES
CRSLDPFKPFIIFSNRHEIRRIDLHKGDYSVLVPGLRNTIALDFHLSQSALYWTDVVEDK
IYRGKLLDNGALTSFEVVIQYGLATPEGLAVDWIAGNIYWVESNLDQIEVAKLDGTLRTT
LLAGDIEHPRAIALDPRDGILFWTDWDASLPRIEAASMSGAGRRTVHRETGSGGWPNGLT
VDYLEKRILWIDARSDAIYSARYDGSGHMEVLRGHEFLSHPFAVTLYGGEVYWTDWRTNT
LAKANKWTGHNVTVVQRTNTQPFDLQVYHPSRQPMAPNPCEANGGQGPCSHLCLINYNRT
VSCACPHLMKLHKDNTTCYEFKKFLLYARQMEIRGVDLDAPYYNYIISFTVPDIDNVTVL
DYDAREQRVYWSDVRTQAIKRAFINGTGVETVVSADLPNAHGLAVDWVSRNLFWTSYDTN
KKQINVARLDGSFKNAVVQGLEQPHGLVVHPLRGKLYWTDGDNISMANMDGSNRTLLFSG
QKGPVGLAIDFPESKLYWISSGNHTINRCNLDGSGLEVIDAMRSQLGKATALAIMGDKLW
WADQVSEKMGTCSKADGSGSVVLRNSTTLVMHMKVYDESIQLDHKGTNPCSVNNGDCSQL
CLPTSETTRSCMCTAGYSLRSGQQACEGVGSFLLYSVHEGIRGIPLDPNDKSDALVPVSG
TSLAVGIDFHAENDTIYWVDMGLSTISRAKRDQTWREDVVTNGIGRVEGIAVDWIAGNIY
WTDQGFDVIEVARLNGSFRYVVISQGLDKPRAITVHPEKGYLFWTEWGQYPRIERSRLDG
TERVVLVNVSISWPNGISVDYQDGKLYWCDARTDKIERIDLETGENREVVLSSNNMDMFS
VSVFEDFIYWSDRTHANGSIKRGSKDNATDSVPLRTGIGVQLKDIKVFNRDRQKGTNVCA
VANGGCQQLCLYRGRGQRACACAHGMLAEDGASCREYAGYLLYSERTILKSIHISDERNL
NAPVQPFEDPEHMKNVIALAFDYRAGTSPGTPNRIFFSDIHFGNIQQINDDGSRRITIVE
NVGSVEGLAYHRGWDTLYWTSYTTSTITRHTVDQTRPGAFERETVITMSGDDHPRAFVLD
ECQNLMFWTNWNEQHPSIMRAALSGANVLTLIEKDIRTPNGLAIDHRAEKLYFSDATLDK
IERCEYDGSHRYVILKSEPVHPFGLAVYGEHIFWTDWVRRAVQRANKHVGSNMKLLRVDI
PQQPMGIIAVANDTNSCELSPCRINNGGCQDLCLLTHQGHVNCSCRGGRILQDDLTCRAV
NSSCRAQDEFECANGECINFSLTCDGVPHCKDKSDEKPSYCNSRRCKKTFRQCSNGRCVS
NMLWCNGADDCGDGSDEIPCNKTACGVGEFRCRDGTCIGNSSRCNQFVDCEDASDEMNCS
ATDCSSYFRLGVKGVLFQPCERTSLCYAPSWVCDGANDCGDYSDERDCPGVKRPRCPLNY
FACPSGRCIPMSWTCDKEDDCEHGEDETHCNKFCSEAQFECQNHRCISKQWLCDGSDDCG
DGSDEAAHCEGKTCGPSSFSCPGTHVCVPERWLCDGDKDCADGADESIAAGCLYNSTCDD
REFMCQNRQCIPKHFVCDHDRDCADGSDESPECEYPTCGPSEFRCANGRCLSSRQWECDG
ENDCHDQSDEAPKNPHCTSPEHKCNASSQFLCSSGRCVAEALLCNGQDDCGDSSDERGCH
INECLSRKLSGCSQDCEDLKIGFKCRCRPGFRLKDDGRTCADVDECSTTFPCSQRCINTH
GSYKCLCVEGYAPRGGDPHSCKAVTDEEPFLIFANRYYLRKLNLDGSNYTLLKQGLNNAV
ALDFDYREQMIYWTDVTTQGSMIRRMHLNGSNVQVLHRTGLSNPDGLAVDWVGGNLYWCD
KGRDTIEVSKLNGAYRTVLVSSGLREPRALVVDVQNGYLYWTDWGDHSLIGRIGMDGSSR
SVIVDTKITWPNGLTLDYVTERIYWADAREDYIEFASLDGSNRHVVLSQDIPHIFALTLF
EDYVYWTDWETKSINRAHKTTGTNKTLLISTLHRPMDLHVFHALRQPDVPNHPCKVNNGG
CSNLCLLSPGGGHKCACPTNFYLGSDGRTCVSNCTASQFVCKNDKCIPFWWKCDTEDDCG
DHSDEPPDCPEFKCRPGQFQCSTGICTNPAFICDGDNDCQDNSDEANCDIHVCLPSQFKC
TNTNRCIPGIFRCNGQDNCGDGEDERDCPEVTCAPNQFQCSITKRCIPRVWVCDRDNDCV
DGSDEPANCTQMTCGVDEFRCKDSGRCIPARWKCDGEDDCGDGSDEPKEECDERTCEPYQ
FRCKNNRCVPGRWQCDYDNDCGDNSDEESCTPRPCSESEFSCANGRCIAGRWKCDGDHDC
ADGSDEKDCTPRCDMDQFQCKSGHCIPLRWRCDADADCMDGSDEEACGTGVRTCPLDEFQ
CNNTLCKPLAWKCDGEDDCGDNSDENPEECARFVCPPNRPFRCKNDRVCLWIGRQCDGTD
NCGDGTDEEDCEPPTAHTTHCKDKKEFLCRNQRCLSSSLRCNMFDDCGDGSDEEDCSIDP
KLTSCATNASICGDEARCVRTEKAAYCACRSGFHTVPGQPGCQDINECLRFGTCSQLCNN
TKGGHLCSCARNFMKTHNTCKAEGSEYQVLYIADDNEIRSLFPGHPHSAYEQAFQGDESV
RIDAMDVHVKAGRVYWTNWHTGTISYRSLPPAAPPTTSNRHRRQIDRGVTHLNISGLKMP
RGIAIDWVAGNVYWTDSGRDVIEVAQMKGENRKTLISGMIDEPHAIVVDPLRGTMYWSDW
GNHPKIETAAMDGTLRETLVQDNIQWPTGLAVDYHNERLYWADAKLSVIGSIRLNGTDPI
VAADSKRGLSHPFSIDVFEDYIYGVTYINNRVFKIHKFGHSPLVNLTGGLSHASDVVLYH
QHKQPEVTNPCDRKKCEWLCLLSPSGPVCTCPNGKRLDNGTCVPVPSPTPPPDAPRPGTC
NLQCFNGGSCFLNARRQPKCRCQPRYTGDKCELDQCWEHCRNGGTCAASPSGMPTCRCPT
GFTGPKCTQQVCAGYCANNSTCTVNQGNQPQCRCLPGFLGDRCQYRQCSGYCENFGTCQM
AADGSRQCRCTAYFEGSRCEVNKCSRCLEGACVVNKQSGDVTCNCTDGRVAPSCLTCVGH
CSNGGSCTMNSKMMPECQCPPHMTGPRCEEHVFSQQQPGHIASILIPLLLLLLLVLVAGV
VFWYKRRVQGAKGFQHQRMTNGAMNVEIGNPTYKMYEGGEPDDVGGLLDADFALDPDKPT
NFTNPVYATLYMGGHGSRHSLASTDEKRELLGRGPEDEIGDPLA
>Biotin Acceptor Peptide (BAP)
SEQ ID NO: 77
LNDIFEAQKIEWH
>Lipoate Acceptor Peptide 2 (LAP2)
SEQ ID NO: 78
GFEIDKVWYDLDA
>HAPylation motif, n = 1 to 400
SEQ ID NO: 82
(Gly4Ser)n
>CTP
SEQ ID NO: 82
DSSSSKAPPPSLPSPSRLPGPSDTPILPQ
>FVII-PABC peptide
SEQ ID NO: 79
Biotin-Pra-GGGG-DPhe-Pip-Arg-PABC-IVGGKV-COSBn, Pra =
L-Propargyiglycine
>FX-PABC peptide
SEQ ID NO: 80
GG-DPhe-Pip-Arg-PABC-IVGGQE-COSBn
>SYN470
SEQ ID NO: 81
IKPEAPGEDASPEELNRYYASLRHYLNLVTRQRY-PEG4-G1y-COSBn

1. A procoagulant compound having a formula:
(Het2)-(Pep2)-(Het1)-(L)-Zy-Bx-Pep1
wherein,
Het1 is a first heterologous molecule, which is either absent or present;
Het2 is a second heterologous molecule, which is either absent or present;
L is a linker, which is either absent or present;
Zy is a protease-cleavable substrate;
Bx is a self-immolative spacer;
Pep1 is a polypeptide; and,
Pep2 is a polypeptide, which is either absent or present;
wherein, Pep1 or Pep2 comprises a clotting factor or a fragment thereof, or a synthetic procoagulant peptide.

2. The procoagulant compound according to claim 1, wherein the self-immolative spacer undergoes 1,4 elimination or 1,6 elimination after the enzymatic cleavage of the protease-cleavable substrate.

3. (canceled)

4. The procoagulant compound according to claim 1, wherein the self-immolative spacer is a p-aminobenzyl carbamate (PABC), a p-aminobenzyl ether (PABE), or a p-aminobenzyl carbonate.

5-10. (canceled)

11. The procoagulant compound according to claim 1, wherein the protease-cleavable substrate comprises a coagulation cascade protease substrate.

12-16. (canceled)

17. The procoagulant compound according to claim 1, wherein the protease-cleavable substrate comprises a cleavage site for a protease selected from the group consisting of neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases. enamelysin, tumor necrosis factor α-converting enzyme, insulysin, nardilysin, mitochondrial processing peptidase, magnolysin, dactylysin-like metal loproteases, neutrophil collagenase, matrix metallopeptidases, membrane-type matrix metalloproteinases, SP2 endopeptidase, prostate specific antigen (PSA), plasmin, urokinase, human fibroblast activation protein (FAPα), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose-binding protein-associated serine protease, coagulation factors, thrombin, protein c, u and t-type plasminogen activator, cathepsin G, hepsin, prostasin, hepatocyte growth factor-activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin D, pepsins, chymosyn, gastricsin, renin, yapsin and/or memapsins, and Prostate-Specific antigen (PSA).

18-21. (canceled)

22. The procoagulant compound according to claim 1, wherein Pep1 is a clotting factor or a fragment thereof, Pep2 is a clotting factor or a fragment thereof or both Pep1 and Pep2 are clotting factors or fragments thereof.

23. The procoagulant compound according to claim 1, wherein Pep1 is a heavy chain of a clotting factor and Pep2 is a light chain of the clotting factor.

24-26. (canceled)

27. The procoagulant compound according to claim 1, wherein Pep1 is a synthetic procoagulant peptide, Pep2 is a synthetic procoagulant peptide or both Pep1 and Pep2 are synthetic procoagulant peptides.

28. The procoagulant compound according to claim 1, wherein the linker comprises a peptide linker or a non-peptide linker.

29-34. (canceled)

35. The procoagulant compound according to claim 1, wherein the heterologous moiety comprises a half-life extending moiety.

36-46. (canceled)

47. The procoagulant compound according to claim 1, wherein the heterologous moiety comprises a non-peptidic active agent, a targeting or ligand binding moiety or an anchor or scaffolding molecule.

48-52. (canceled)

53. The procoagulant compound according to claim 1, comprising the formula Het-L- Zy-Bx-Pep1, wherein:
Het is cysteine,
L is a peptide linker,
Zy is a synthetic thrombin substrate,
Bx is the self-immolative spacer, and
Pep1 is procoagulant peptide.

54. (canceled)

55. The procoagulant compound according to claim 1, comprising the formula
Zy-Bx-Pep1, wherein:
Zy is a synthetic thrombin substrate,
Bx is a self-immolative spacer, and
Pep1 is a clotting factor.

56-57. (canceled)

58. A pharmaceutical composition comprising a procoagulant compound according to claim 1, and a pharmaceutically acceptable carrier.

59. A method for treating, ameliorating, or preventing a bleeding disease or disorder in a subject, comprising administering to the subject an effective amount of a procoagulant compound according to claim 1.

60-63. (canceled)

64. A method of treating, ameliorating, or preventing a deficiency in at least one blood coagulation factor in mammalian subject, wherein the blood coagulation factor is selected from FV, FVII, FVIIa, FVIII, FIX, FX, FXI, and vWF, the method comprising administering to the subject an effective amount of the procoagulant compound according to any claim 1.

65. (canceled)

66. The procoagulant compound according to claim 1 for treating a subject having a blood coagulation disorder.

67-68. (canceled)

69. A method for making the procoagulant compound according to claim 1, the method comprising using solid-phase peptide synthesis.

70-76. (canceled)

77. A method of activating a procoagulant peptide comprising contacting a procoagulant compound according to claim 1 with a protease specific for the protease-cleavable substrate moiety in said procoagulant compound, wherein the activated procoagulant peptide is released upon proteolytic cleavage of the protease-cleavable substrate moiety.

78-82. (canceled)

83. The procoagulant compound according to claim 1, wherein the self-immolative spacer comprises an exosite binding peptide.

84-113. (canceled)


SEQUENCE LISTING (90 sequences)

SEQ ID NO: 1 - skipped

SEQ ID NO: 2 - protein, 9 aa, Artificial Sequence
  FXIa cleavage site
TQSFNDFTR

SEQ ID NO: 3 - protein, 10 aa, Artificial Sequence
  FXIa cleavage site
SVSQTSKLTR

SEQ ID NO: 4 - protein, 10 aa, Artificial Sequence
  thrombin cleavage site
DFLAEGGGVR

SEQ ID NO: 5 - protein, 7 aa, Artificial Sequence
  thrombin cleavage site
TTKIKPR

SEQ ID NO: 6 - protein, 5 aa, Artificial Sequence
  thrombin cleavage site
LVPRG

SEQ ID NO: 7 - protein, 5 aa, Artificial Sequence
  thrombin cleavage site
ALRPR

SEQ ID NO: 8 - protein, 12 aa, Artificial Sequence
  synthetic procoagulant peptide
KLTCLASYCWLF

SEQ ID NO: 9 - protein, 22 aa, Artificial Sequence
  synthetic procoagulant peptide
RRAPGKLTCLASYCWLFWTGIA

SEQ ID NO: 10 - protein, 22 aa, Artificial Sequence
  synthetic procoagulant peptide
RRAPGKLQCLASYCWLFWTGIA

SEQ ID NO: 11 - protein, 32 aa, Artificial Sequence
  synthetic procoagulant peptide
PRIRTVGPGSRSASGKLTCLASYCWLFWTGIA

SEQ ID NO: 12 - protein, 32 aa, Artificial Sequence
  synthetic procoagulant peptide
SKQGRPISPDRRAAGKLTCLASYCWLFWTGIA

SEQ ID NO: 13 - protein, 32 aa, Artificial Sequence
  synthetic procoagulant peptide
PRIRTVGPGSRSASGKSTCLASYCWLFWTGIA

SEQ ID NO: 14 - protein, 32 aa, Artificial Sequence
  synthetic procoagulant peptide
SRIRTVSPGSRSASGKSTCLASYCWLFWTGIA

SEQ ID NO: 15 - protein, 32 aa, Artificial Sequence
  synthetic procoagulant peptide
PRSRTVGPGSRSASGKSTCLASYCWLFWTGIA

SEQ ID NO: 16 - protein, 10 aa, Artificial Sequence
  thrombin-cleavable substrate
ALRPRVVGGA

SEQ ID NO: 17 - protein, 5 aa, Artificial Sequence
  thrombin-cleavable substrate
ALVPR

SEQ ID NO: 18 - protein, 4 aa, Artificial Sequence
  thrombin-cleavable substrate
LVPR

SEQ ID NO: 19-24 - skipped (6 entries)

SEQ ID NO: 25 - protein, 7 aa, Artificial Sequence
  protease-cleavable substrate
  SITE (4)..(5): FXIa cleavage site
KLTRAET

SEQ ID NO: 26 - protein, 7 aa, Artificial Sequence
  protease-cleavable substrate
  SITE (4)..(5): FXIa cleavage site
DFTRVVG

SEQ ID NO: 27 - protein, 8 aa, Artificial Sequence
  protease-cleavable substrate
  SITE (4)..(5): FXIIa cleavage site
TMTRIVGG

SEQ ID NO: 28 - protein, 8 aa, Artificial Sequence
  protease-cleavable substrate
  SITE (4)..(5): kallikrein cleavage site
SPFRSTGG

SEQ ID NO: 29 - protein, 8 aa, Artificial Sequence
  protease-cleavable substrate
  SITE (4)..(5): FVIIa cleavage site
LQVRIVGG

SEQ ID NO: 30 - protein, 8 aa, Artificial Sequence
  protease-cleavable substrate
  SITE (4)..(5): FIXa cleavage site
PLGRIVGG

SEQ ID NO: 31 - protein, 8 aa, Artificial Sequence
  protease-cleavable substrate
  SITE (4)..(5): FXa cleavage site
IEGRTVGG

SEQ ID NO: 32 - protein, 8 aa, Artificial Sequence
  protease-cleavable substrate
  SITE (4)..(5): FIIa (thrombin) cleavage site
LTPRSLLV

SEQ ID NO: 33 - protein, 8 aa, Artificial Sequence
  protease-cleavable substrate
  SITE (4)..(5): Elastase-2 cleavage site
LGPVSGVP

SEQ ID NO: 34 - protein, 8 aa, Artificial Sequence
  protease-cleavable substrate
  SITE (4)..(5): Granzyme-B cleavage site
VAGDSLEE

SEQ ID NO: 35 - protein, 8 aa, Artificial Sequence
  protease-cleavable substrate
  SITE (4)..(5): MMP-12 cleavage site
GPAGLGGA

SEQ ID NO: 36 - protein, 8 aa, Artificial Sequence
  protease-cleavable substrate
  SITE (4)..(5): MMP-13 cleavage site
GPAGLRGA

SEQ ID NO: 37 - protein, 8 aa, Artificial Sequence
  protease-cleavable substrate
  SITE (4)..(5): MMP-17 cleavage site
APLGLRLR

SEQ ID NO: 38 - protein, 8 aa, Artificial Sequence
  protease-cleavable substrate
  SITE (4)..(5): MMP-20 cleavage site
PALPLVAQ

SEQ ID NO: 39 - protein, 7 aa, Artificial Sequence
  protease-cleavable substrate
  SITE (6)..(7): TEV cleavage site
ENLYFQG

SEQ ID NO: 40 - protein, 8 aa, Artificial Sequence
  protease-cleavable substrate
  SITE (4)..(5): Enterokinase cleavage site
DDDKIVGG

SEQ ID NO: 41 - protein, 8 aa, Artificial Sequence
  protease-cleavable substrate
  SITE (6)..(7): Protease 3C (PRESCISSION) cleavage site
LEVLFQGP

SEQ ID NO: 42 - protein, 8 aa, Artificial Sequence
  protease-cleavable substrate
  SITE (4)..(5): Sortase A cleavage site
LPKTGSES

SEQ ID NO: 43 - protein, 4 aa, Artificial Sequence
  peptide linker
  REPEAT (1)..(4): Repeat from 1-100
GGGS

SEQ ID NO: 44 - protein, 8 aa, Artificial Sequence
  peptide linker
  REPEAT (1)..(3): Repeat 1-100
  REPEAT (4)..(8): Repeat 1-100
GGSGGGGS

SEQ ID NO: 45 - protein, 7 aa, Artificial Sequence
  peptide linker
SGGSGGS

SEQ ID NO: 46 - protein, 15 aa, Artificial Sequence
  peptide linker
GGSGGSGGSGGSGGG

SEQ ID NO: 47 - protein, 16 aa, Artificial Sequence
  peptide linker
GGSGGSGGGGSGGGGS

SEQ ID NO: 48 - protein, 18 aa, Artificial Sequence
  peptide linker
GGSGGSGGSGGSGGSGGS

SEQ ID NO: 49 - protein, 15 aa, Artificial Sequence
  peptide linker
GGGGSGGGGSGGGGS

SEQ ID NO: 50 - protein, 4 aa, Artificial Sequence
  peptide linker
  REPEAT (1)..(4): Repeat from 1-100
GGGG

SEQ ID NO: 51 - protein, 32 aa, Artificial Sequence
  CTP Peptide
DPRFQDSSSSKAPPPSLPSPSRLPGPSDTPIL

SEQ ID NO: 52 - protein, 28 aa, Artificial Sequence
  CTP peptide
SSSSKAPPPSLPSPSRLPGPSDTPILPQ

SEQ ID NO: 53 - protein, 11 aa, Artificial Sequence
  albumin-binding peptides
DICLPRWGCLW

SEQ ID NO: 54 - protein, 18 aa, Artificial Sequence
  albumin-binding peptide
RLIEDICLPRWGCLWEDD

SEQ ID NO: 55 - protein, 20 aa, Artificial Sequence
  albumin-binding peptide
QRLMEDICLPRWGCLWEDDF

SEQ ID NO: 56 - protein, 21 aa, Artificial Sequence
  albumin-binding peptide
QGLIGDICLPRWGCLWGDSVK

SEQ ID NO: 57 - protein, 20 aa, Artificial Sequence
  albumin-binding peptide
GEWWEDICLPRWGCLWEEED

SEQ ID NO: 58 - protein, 20 aa, Artificial Sequence
  PAS Sequence
ASPAAPAPASPAAPAPSAPA

SEQ ID NO: 59 - protein, 20 aa, Artificial Sequence
  PAS Sequence
AAPASPAPAAPSAPAPAAPS

SEQ ID NO: 60 - protein, 20 aa, Artificial Sequence
  PAS sequence
APSSPSPSAPSSPSPASPSS

SEQ ID NO: 61 - protein, 19 aa, Artificial Sequence
  PAS sequence
APSSPSPSAPSSPSPASPS

SEQ ID NO: 62 - protein, 20 aa, Artificial Sequence
  PAS sequence
SSPSAPSPSSPASPSPSSPA

SEQ ID NO: 63 - protein, 24 aa, Artificial Sequence
  PAS Sequence
AASPAAPSAPPAAASPAAPSAPPA

SEQ ID NO: 64 - protein, 20 aa, Artificial Sequence
  PAS Sequence
ASAAAPAAASAAASAPSAAA

SEQ ID NO: 65 - DNA, 6408 bp, Artificial Sequence
  FVII-186
aagcttgccgccaccatggtctcccaggccctcaggctcctctgccttctgcttgggcttttcgaacggcggtggtaccagagggtccgggagtccgaggagacggaagacgaacccgaacagggctgcctggctgcagtcttcgtaacccaggaggaagcccacggcgtcctgcaccgggtcccgacggaccgacgtcagaagcattgggtcctccttcgggtgccgcaggacgtggcccgccggcgcgccaacgcgttcctggaggagctgcggccgggctccctggagagggagtgcgcggccgcgcggttgcgcaaggacctcctcgacgccggcccgagggacctctccctcacgaaggaggagcagtgctccttcgaggaggcccgggagatcttcaaggacgcggagaggacgttcctcctcgtcacgaggaagctcctccgggccctctagaagttcctgcgcctctcctgcaagctgttctggatttcttacagtgatggggaccagtgtgcctcaagtccatgccagaatttcgacaagacctaaagaatgtcactacccctggtcacacggagttcaggtacggtcttagggggctcctgcaaggaccagctccagtcctatatctgcttctgcctccctgccttcgagcccccgaggacgttcctggtcgaggtcaggatatagacgaagacggagggacggaagctcggccggaactgtgagacgcacaaggatgaccagctgatctgtgtgaacgagaacggcggcccggccttgacactctgcgtgttcctactggtcgactagacacacttgctcttgccgccgtgtgagcagtactgcagtgaccacacgggcaccaagcgctcctgtcggtgccacgaggggacactcgtcatgacgtcactggtgtgcccgtggttcgcgaggacagccacggtgctcccctactctctgctggcagacggggtgtcctgcacacccacagttgaatatccatgtggaaaaatgagagacgaccgtctgccccacaggacgtgtgggtgtcaacttataggtacaccttttatacctattctagaaaaaagaaatgccagcaaaccccaaggccgaaagaggaggaagaggtatggataagatcttttttctttacggtcgtttggggttccggctttctcctccttctccggtggcggcggatcaggtgggggtggatcaggcggtggaggttccctgcaggactcagaaccaccgccgcctagtccacccccacctagtccgccacctccaagggacgtcctgagtcttgtcaatcaagaagctaagccagaggtcaagccagaagtcaagcctgagactcacatcaatcagttagttcttcgattcggtctccagttcggtcttcagttcggactctgagtgtagttattaaaggtgtccgatggatcttcagagatcttcttcaagatcaaaaagaccactcctttaaatttccacaggctacctagaagtctctagaagaagttctagtttttctggtgaggaaatagaaggctgatggaagcgttcgctaaaagacagggtaaggaaatggactccttaagattctcttccgactaccttcgcaagcgattttctgtcccattcctttacctgaggaattctaagttgtacgacggtattagaattcaagctgatcaggcccctgaagatttggacatggaggataacatgctgccataatcttaagttcgactagtccggggacttctaaacctgtacctcctaaacgatattattgaggctcaccgcgaacagattggaggttgccccaaaggggagtgtccattgctataataactccgagtggcgcttgtctaacctccaacggggtttcccctcacaggttggcaggtcctgttgttggtgaatggagctcagttgtgtggggggaccctgatcaacaccaccgtccaggacaacaaccacttacctcgagtcaacacacccccctgggactagttgtggatctgggtggtctccgcggcccactgtttcgacaaaatcaagaactggaggaacctgatctagacccaccagaggcgccgggtgacaaagctgttttagttcttgacctccttggactaggcggtgctgggcgagcacgacctcagcgagcacgacggggatgagcagagccggcgggtgcgccacgacccgctcgtgctggagtcgctcgtgctgcccctactcgtctcggccgcccacgcgcaggtcatcatccccagcacgtacgtcccgggcaccaccaaccacgacatcgcgctgcgcgtccagtagtaggggtcgtgcatgcagggcccgtggtggttggtgctgtagcgcgacctccgcctgcaccagcccgtggtcctcactgaccatgtggtgcccctctgcctgcccgaagaggcggacgtggtcgggcaccaggagtgactggtacaccacggggagacggacgggcttcggacgttctctgagaggacgctggccttcgtgcgcttctcattggtcagcggctggggcgcctgcaagagactctcctgcgaccggaagcacgcgaagagtaaccagtcgccgaccccgcagctgctggaccgtggcgccacggccctggagctcatggtcctcaacgtgccccggctggtcgacgacctggcaccgcggtgccgggacctcgagtaccaggagttgcacggggccgacatgacccaggactgcctgcagcagtcacggaaggtgggagactccccaaatatcacggagtactgggtcctgacggacgtcgtcagtgccttccaccctctgaggggtttatagtgcctctacatgttctgtgccggctactcggatggcagcaaggactcctgcaagggggacagtggaatgtacaagacacggccgatgagcctaccgtcgttcctgaggacgttccccctgtcacctggcccacatgccacccactaccggggcacgtggtacctgacgggcatcgtcagctggggcccgggtgtacggtgggtgatggccccgtgcaccatggactgcccgtagcagtcgaccccgcagggctgcgcaaccgtgggccactttggggtgtacaccagggtctcccagtacatcgaggtcccgacgcgttggcacccggtgaaaccccacatgtggtcccagagggtcatgtagctctggctgcaaaagctcatgcgctcagagccacgcccaggagtcctcctgcgagccccatttaccgacgttttcgagtacgcgagtctcggtgcgggtcctcaggaggacgctcggggtaaacccggtggcggtggctccggcggaggtgggtccggtggcggcggatcaggtgggggtggagggccaccgccaccgaggccgcctccacccaggccaccgccgcctagtccacccccaccttcaggcggtggaggttccggtggcgggggatccgacaaaactcacacatgcccaccgtgcagtccgccacctccaaggccaccgccccctaggctgttttgagtgtgtacgggtggcacgccagctccggaactcctgggcggaccgtcagtcttcctcttccccccaaaacccaaggacggtcgaggccttgaggacccgcctggcagtcagaaggagaaggggggttttgggttcctgaccctcatgatctcccggacccctgaggtcacatgcgtggtggtggacgtgagccacgaatgggagtactagagggcctggggactccagtgtacgcaccaccacctgcactcggtgcttgaccctgaggtcaagttcaactggtacgtggacggcgtggaggtgcataatgccaagacactgggactccagttcaagttgaccatgcacctgccgcacctccacgtattacggttctgtaagccgcgggaggagcagtacaacagcacgtaccgtgtggtcagcgtcctcaccgtcctgttcggcgccctcctcgtcatgttgtcgtgcatggcacaccagtcgcaggagtggcaggaccaccaggactggctgaatggcaaggagtacaagtgcaaggtctccaacaaagccctcccagtggtcctgaccgacttaccgttcctcatgttcacgttccagaggttgtttcgggagggtgcccccatcgagaaaaccatctccaaagccaaagggcagccccgagaaccacaggtgtaccgggggtagctcttttggtagaggtttcggtttcccgtcggggctcttggtgtccacatgaccctgcccccatcccgggatgagctgaccaagaaccaggtcagcctgacctgcctggtctgggacgggggtagggccctactcgactggttcttggtccagtcggactggacggaccagaaaggcttctatcccagcgacatcgccgtggagtgggagagcaatgggcagccggagaactttccgaagatagggtcgctgtagcggcacctcaccctctcgttacccgtcggcctcttgaactacaagaccacgcctcccgtgttggactccgacggctccttcttcctctacagcaagttgatgttctggtgcggagggcacaacctgaggctgccgaggaagaaggagatgtcgttcctcaccgtggacaagagcaggtggcagcaggggaacgtcttctcatgctccgtgatgcatgagtggcacctgttctcgtccaccgtcgtccccttgcagaagagtacgaggcactacgtagaggctctgcacaaccactacacgcagaagagcctctccctgtctccgggtaaaggtggcctccgagacgtgttggtgatgtgcgtcttctcggagagggacagaggcccatttccaccgggcggatcaggtgggggtggatcaggcggtggaggttccggtggcgggggatcagacaaaccgcctagtccacccccacctagtccgccacctccaaggccaccgccccctagtctgtttactcacacatgcccaccgtgcccagcacctgaactcctgggaggaccgtcagtcttcctctgagtgtgtacgggtggcacgggtcgtggacttgaggaccctcctggcagtcagaaggagttccccccaaaacccaaggacaccctcatgatctcccggacccctgaggtcacatgcgtgaaggggggttttgggttcctgtgggagtactagagggcctggggactccagtgtacgcacgtggtggacgtgagccacgaagaccctgaggtcaagttcaactggtacgtggacggcgtgcaccacctgcactcggtgcttctgggactccagttcaagttgaccatgcacctgccgcacgaggtgcataatgccaagacaaagccgcgggaggagcagtacaacagcacgtaccgtgtgctccacgtattacggttctgtttcggcgccctcctcgtcatgttgtcgtgcatggcacacgtcagcgtcctcaccgtcctgcaccaggactggctgaatggcaaggagtacaagtgcaagcagtcgcaggagtggcaggacgtggtcctgaccgacttaccgttcctcatgttcacgttcgtctccaacaaagccctcccagcccccatcgagaaaaccatctccaaagccaaagggcagcagaggttgtttcgggagggtcgggggtagctcttttggtagaggtttcggtttcccgtcccccgagaaccacaggtgtacaccctgcccccatcccgcgatgagctgaccaagaaccagggggctcttggtgtccacatgtgggacgggggtagggcgctactcgactggttcttggtcgtcagcctgacctgcctggtcaaaggcttctatcccagcgacatcgccgtggagtgggagcagtcggactggacggaccagtttccgaagatagggtcgctgtagcggcacctcaccctcagcaatgggcagccggagaacaactacaagaccacgcctcccgtgttggactccgacggctcgttacccgtcggcctcttgttgatgttctggtgcggagggcacaacctgaggctgccgtccttcttcctctacagcaagctcaccgtggacaagagcaggtggcagcaggggaacgtcaggaagaaggagatgtcgttcgagtggcacctgttctcgtccaccgtcgtccccttgcagttctcatgctccgtgatgcatgaggctctgcacaaccactacacgcagaagagcctctccaagagtacgaggcactacgtactccgagacgtgttggtgatgtgcgtcttctcggagaggctgtctccgggtaaatgagaattcgacagaggcccatttactcttaag

SEQ ID NO: 66 - protein, 1060 aa, Artificial Sequence
  FVII-186
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRKRRKRGGGGSGGGGSGGGGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 6046 bp, Artificial Sequence
  FX-011
aagcttatgggtcgtccactgcacctcgtcctgctcagtgcctccctggctggcctcctgttcgaatacccagcaggtgacgtggagcaggacgagtcacggagggaccgaccggaggacctgctcggggaaagtctgttcatccgcagggagcaggccaacaacatcctggcgagggtcgacgagcccctttcagacaagtaggcgtccctcgtccggttgttgtaggaccgctcccagaggagggccaattcctttcttgaagagatgaagaaaggacacctcgaaagagagtgcatgtcctcccggttaaggaaagaacttctctacttctttcctgtggagctttctctcacgtacgaagagacctgctcatacgaagaggcccgcgaggtctttgaggacagcgacaagacgaatcttctctggacgagtatgcttctccgggcgctccagaaactcctgtcgctgttctgcttagaattctggaataaatacaaagatggcgaccagtgtgagaccagtccttgccagaaccagcttaagaccttatttatgtttctaccgctggtcacactctggtcaggaacggtcttggtcggcaaatgtaaagacggcctcggggaatacacctgcacctgtttagaaggattcgaaggcccgtttacatttctgccggagccccttatgtggacgtggacaaatcttcctaagcttccgaaaaactgtgaattattcacacggaagctctgcagcctggacaacggggactgtgaccagtttttgacacttaataagtgtgccttcgagacgtcggacctgttgcccctgacactggtcttctgccacgaggaacagaactctgtggtgtgctcctgcgcccgcgggtacaccctggctaagacggtgctccttgtcttgagacaccacacgaggacgcgggcgcccatgtgggaccgagacaacggcaaggcctgcattcccacagggccctacccctgtgggaaacagaccctggaactgttgccgttccggacgtaagggtgtcccgggatggggacaccctttgtctgggaccttcgcaggaagaggtcagtggcccaggccaccagcagcagcggggaggcccctgacagcatcgcgtccttctccagtcaccgggtccggtggtcgtcgtcgcccctccggggactgtcgtagacatggaagccatatgatgcagccgacctggaccccaccgagaaccccttcgacctgctttgtaccttcggtatactacgtcggctggacctggggtggctcttggggaagctggacgaagacttcaaccagacgcagcctgagaggggcgacaacaacggtggcggcggatcaggtgggctgaagttggtctgcgtcggactctccccgctgttgttgccaccgccgcctagtccacccggtggatcaggcggtggaggttccggtggcgggggatccaggaagaggaggaagaggtgcccacctagtccgccacctccaaggccaccgccccctaggtccttctcctccttctccacgaaggacggggagtgtccctggcaggccctgctcatcaatgaggaaaacgagggtttttgtttcctgcccctcacagggaccgtccgggacgagtagttactccttttgctcccaaaaacaggaggtaccattctgagcgagttctacatcctaacggcagcccactgtctctaccaagcccctccatggtaagactcgctcaagatgtaggattgccgtcgggtgacagagatggttcggaagagattcaaggtgagggtaggggaccggaacacggagcaggaggagggcggtgaggcgttctctaagttccactcccatcccctggccttgtgcctcgtcctcctcccgccactccgcgtgcacgaggtggaggtggtcatcaagcacaaccggttcacaaaggagacctatgacttccacgtgctccacctccaccagtagttcgtgttggccaagtgtttcctctggatactgaaggacatcgccgtgctccggctcaagacccccatcaccttccgcatgaacgtggcgcctgccctgtagcggcacgaggccgagttctgggggtagtggaaggcgtacttgcaccgcggacggtgcctccccgagcgtgactgggccgagtccacgctgatgacgcagaagacggggattgtgacggaggggctcgcactgacccggctcaggtgcgactactgcgtcttctgcccctaacacagcggcttcgggcgcacccacgagaagggccggcagtccaccaggctcaagatgctggagtcgccgaagcccgcgtgggtgctcttcccggccgtcaggtggtccgagttctacgacctcgtgccctacgtggaccgcaacagctgcaagctgtccagcagcttcatcatcacccagaaccacgggatgcacctggcgttgtcgacgttcgacaggtcgtcgaagtagtagtgggtcttgatgttctgtgccggctacgacaccaagcaggaggatgcctgccagggggacagcgggggctacaagacacggccgatgctgtggttcgtcctcctacggacggtccccctgtcgcccccgccgcacgtcacccgcttcaaggacacctacttcgtgacaggcatcgtcagctggggagagggcgtgcagtgggcgaagttcctgtggatgaagcactgtccgtagcagtcgacccctctcggctgtgcccgtaaggggaagtacgggatctacaccaaggtcaccgccttcctcaagtggccgacacgggcattccccttcatgccctagatgtggttccagtggcggaaggagttcaccatcgacaggtccatgaaaaccaggggcttgcccaaggccaagagccatgccccggaggtctagctgtccaggtacttttggtccccgaacgggttccggttctcggtacggggcctccagataacgtcctctccattaaaagaagaccaagtagatccgcggctcattgatggtaaggactattgcaggagaggtaattttcttctggttcatctaggcgccgagtaactaccattcctgaaaactcacacatgcccaccgtgcccagctccggaactcctgggaggaccgtcagtcttcttttgagtgtgtacgggtggcacgggtcgaggccttgaggaccctcctggcagtcagaagctcttccccccaaaacccaaggacaccctcatgatctcccggacccctgaggtcacatgcgagaaggggggttttgggttcctgtgggagtactagagggcctggggactccagtgtacggtggtggtggacgtgagccacgaagaccctgaggtcaagttcaactggtacgtggacggccaccaccacctgcactcggtgcttctgggactccagttcaagttgaccatgcacctgccggtggaggtgcataatgccaagacaaagccgcgggaggagcagtacaacagcacgtaccgtcacctccacgtattacggttctgtttcggcgccctcctcgtcatgttgtcgtgcatggcagtggtcagcgtcctcaccgtcctgcaccaggactggctgaatggcaaggagtacaagtgccaccagtcgcaggagtggcaggacgtggtcctgaccgacttaccgttcctcatgttcacgaaggtctccaacaaagccctcccagcccccatcgagaaaaccatctccaaagccaaagggttccagaggttgtttcgggagggtcgggggtagctcttttggtagaggtttcggtttccccagccccgagaaccacaggtgtacaccctgcccccatcccgggatgagctgaccaagaacgtcggggctcttggtgtccacatgtgggacgggggtagggccctactcgactggttcttgcaggtcagcctgacctgcctggtcaaaggcttctatcccagcgacatcgccgtggagtgggtccagtcggactggacggaccagtttccgaagatagggtcgctgtagcggcacctcaccgagagcaatgggcagccggagaacaactacaagaccacgcctcccgtgttggactccgacctctcgttacccgtcggcctcttgttgatgttctggtgcggagggcacaacctgaggctgggctccttcttcctctacagcaagctcaccgtcgacaagagcaggtggcagcaggggaacccgaggaagaaggagatgtcgttcgagtggcagctgttctcgtccaccgtcgtccccttggtcttctcatgctccgtgatgcatgaggctctgcacaaccactacacgcagaagagcctccagaagagtacgaggcactacgtactccgagacgtgttggtgatgtgcgtcttctcggagtccctgtctccgggtaaacggcgccgccggagcggtggcggcggatcaggtgggggtggaagggacagaggcccatttgccgcggcggcctcgccaccgccgcctagtccacccccaccttcaggcggtggaggttccggtggcgggggatccaggaagaggaggaagagggacaaaactagtccgccacctccaaggccaccgccccctaggtccttctcctccttctccctgttttgacacacatgcccaccgtgcccagcaccggaactcctgggcggaccgtcagtcttcctcttcgtgtgtacgggtggcacgggtcgtggccttgaggacccgcctggcagtcagaaggagaagcccccaaaacccaaggacaccctcatgatctcccggacccctgaggtcacatgcgtggtggggggttttgggttcctgtgggagtactagagggcctggggactccagtgtacgcaccacgtggacgtgagccacgaagaccctgaggtcaagttcaactggtacgtggacggcgtggagcacctgcactcggtgcttctgggactccagttcaagttgaccatgcacctgccgcacctcgtgcataatgccaagacaaagccgcgggaggagcagtacaacagcacgtaccgtgtggtccacgtattacggttctgtttcggcgccctcctcgtcatgttgtcgtgcatggcacaccagagcgtcctcaccgtcctgcaccaggactggctgaatggcaaggagtacaagtgcaaggtctcgcaggagtggcaggacgtggtcctgaccgacttaccgttcctcatgttcacgttccagtccaacaaagccctcccagcccccatcgagaaaaccatctccaaagccaaagggcagcccaggttgtttcgggagggtcgggggtagctcttttggtagaggtttcggtttcccgtcgggcgagaaccacaggtgtacaccctgcccccatcccgggatgagctgaccaagaaccaggtcgctcttggtgtccacatgtgggacgggggtagggccctactcgactggttcttggtccagagcctgacctgcctggtcaaaggcttctatcccagcgacatcgccgtggagtgggagagctcggactggacggaccagtttccgaagatagggtcgctgtagcggcacctcaccctctcgaatgggcagccggagaacaactacaagaccacgcctcccgtgttggactccgacggctccttacccgtcggcctcttgttgatgttctggtgcggagggcacaacctgaggctgccgaggttcttcctctacagcaagctcaccgtggacaagagcaggtggcagcaggggaacgtcttcaagaaggagatgtcgttcgagtggcacctgttctcgtccaccgtcgtccccttgcagaagtcatgctccgtgatgcatgaggctctgcacaaccactacacgcagaagagcctctccctgagtacgaggcactacgtactccgagacgtgttggtgatgtgcgtcttctcggagagggactctccgggtaaatgagcggccgcagaggcccatttactcgccggcg

SEQ ID NO: 68 - protein, 1002 aa, Artificial Sequence
  FX-011
MGRPLHLVLLSASLAGLLLLGESLFIRREQANNILARVRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNGGGGSGGGGSGGGGSGGGGSRKRRKRCKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKEDQVDPRLIDGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRRRSGGGGSGGGGSGGGGSGGGGSRKRRKRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 6280 bp, Artificial Sequence
  FX-012
aagcttatgggtcgtccactgcacctcgtcctgctcagtgcctccctggctggcctcctgttcgaatacccagcaggtgacgtggagcaggacgagtcacggagggaccgaccggaggacctgctcggggaaagtctgttcatccgcagggagcaggccaacaacatcctggcgagggtcgacgagcccctttcagacaagtaggcgtccctcgtccggttgttgtaggaccgctcccagaggagggccaattcctttcttgaagagatgaagaaaggacacctcgaaagagagtgcatgtcctcccggttaaggaaagaacttctctacttctttcctgtggagctttctctcacgtacgaagagacctgctcatacgaagaggcccgcgaggtctttgaggacagcgacaagacgaatcttctctggacgagtatgcttctccgggcgctccagaaactcctgtcgctgttctgcttagaattctggaataaatacaaagatggcgaccagtgtgagaccagtccttgccagaaccagcttaagaccttatttatgtttctaccgctggtcacactctggtcaggaacggtcttggtcggcaaatgtaaagacggcctcggggaatacacctgcacctgtttagaaggattcgaaggcccgtttacatttctgccggagccccttatgtggacgtggacaaatcttcctaagcttccgaaaaactgtgaattattcacacggaagctctgcagcctggacaacggggactgtgaccagtttttgacacttaataagtgtgccttcgagacgtcggacctgttgcccctgacactggtcttctgccacgaggaacagaactctgtggtgtgctcctgcgcccgcgggtacaccctggctaagacggtgctccttgtcttgagacaccacacgaggacgcgggcgcccatgtgggaccgagacaacggcaaggcctgcattcccacagggccctacccctgtgggaaacagaccctggaactgttgccgttccggacgtaagggtgtcccgggatggggacaccctttgtctgggaccttcgcaggaagaggggtgggggtggatcaggcggtggaggttccggtggcgggggatcctccgcgtccttctccccacccccacctagtccgccacctccaaggccaccgccccctaggaggctgcaggactcagaagtcaatcaagaagctaagccagaggtcaagccagaagtcaagcctgacgtcctgagtcttcagttagttcttcgattcggtctccagttcggtcttcagttcggagagactcacatcaatttaaaggtgtccgatggatcttcagagatcttcttcaagatcaaactctgagtgtagttaaatttccacaggctacctagaagtctctagaagaagttctagtttaagaccactcctttaagaaggctgatggaagcgttcgctaaaagacagggtaaggaaatgttctggtgaggaaattcttccgactaccttcgcaagcgattttctgtcccattcctttacgactccttaagattcttgtacgacggtattagaattcaagctgatcaggcccctgaagatctgaggaattctaagaacatgctgccataatcttaagttcgactagtccggggacttctattggacatggaggataacgatattattgaggctcaccgcgaacagattggaggttgcaagaacctgtacctcctattgctataataactccgagtggcgcttgtctaacctccaacgttcgacggggagtgtccctggcaggccctgctcatcaatgaggaaaacgagggtttttgtggactgcccctcacagggaccgtccgggacgagtagttactccttttgctcccaaaaacacctggtaccattctgagcgagttctacatcctaacggcagcccactgtctctaccaagccaagccatggtaagactcgctcaagatgtaggattgccgtcgggtgacagagatggttcggttcagattcaaggtgagggtaggggaccggaacacggagcaggaggagggcggtgaggcggtgtctaagttccactcccatcccctggccttgtgcctcgtcctcctcccgccactccgccaccacgaggtggaggtggtcatcaagcacaaccggttcacaaaggagacctatgacttcgacgtgctccacctccaccagtagttcgtgttggccaagtgtttcctctggatactgaagctgatcgccgtgctccggctcaagacccccatcaccttccgcatgaacgtggcgcctgcctgctagcggcacgaggccgagttctgggggtagtggaaggcgtacttgcaccgcggacggacgctccccgagcgtgactgggccgagtccacgctgatgacgcagaagacggggattgtgagcgaggggctcgcactgacccggctcaggtgcgactactgcgtcttctgcccctaacactcgggcttcgggcgcacccacgagaagggccggcagtccaccaggctcaagatgctggaggtgccgaagcccgcgtgggtgctcttcccggccgtcaggtggtccgagttctacgacctccacccctacgtggaccgcaacagctgcaagctgtccagcagcttcatcatcacccagaacatggggatgcacctggcgttgtcgacgttcgacaggtcgtcgaagtagtagtgggtcttgtacttctgtgccggctacgacaccaagcaggaggatgcctgccagggggacagcgggggcccgaagacacggccgatgctgtggttcgtcctcctacggacggtccccctgtcgcccccgggccacgtcacccgcttcaaggacacctacttcgtgacaggcatcgtcagctggggagagggcgtgcagtgggcgaagttcctgtggatgaagcactgtccgtagcagtcgacccctctcccgtgtgcccgtaaggggaagtacgggatctacaccaaggtcaccgccttcctcaagtggatcacacgggcattccccttcatgccctagatgtggttccagtggcggaaggagttcacctaggacaggtccatgaaaaccaggggcttgcccaaggccaagagccatgccccggaggtcatactgtccaggtacttttggtccccgaacgggttccggttctcggtacggggcctccagtatacgtcctctccattaaaagaagaccaagtagatccgcggctcattgatggtaaggacaaatgcaggagaggtaattttcttctggttcatctaggcgccgagtaactaccattcctgtttactcacacatgcccaccgtgcccagctccggaactcctgggaggaccgtcagtcttcctctgagtgtgtacgggtggcacgggtcgaggccttgaggaccctcctggcagtcagaaggagttccccccaaaacccaaggacaccctcatgatctcccggacccctgaggtcacatgcgtgaaggggggttttgggttcctgtgggagtactagagggcctggggactccagtgtacgcacgtggtggacgtgagccacgaagaccctgaggtcaagttcaactggtacgtggacggcgtgcaccacctgcactcggtgcttctgggactccagttcaagttgaccatgcacctgccgcacgaggtgcataatgccaagacaaagccgcgggaggagcagtacaacagcacgtaccgtgtgctccacgtattacggttctgtttcggcgccctcctcgtcatgttgtcgtgcatggcacacgtcagcgtcctcaccgtcctgcaccaggactggctgaatggcaaggagtacaagtgcaagcagtcgcaggagtggcaggacgtggtcctgaccgacttaccgttcctcatgttcacgttcgtctccaacaaagccctcccagcccccatcgagaaaaccatctccaaagccaaagggcagcagaggttgtttcgggagggtcgggggtagctcttttggtagaggtttcggtttcccgtcccccgagaaccacaggtgtacaccctgcccccatcccgggatgagctgaccaagaaccagggggctcttggtgtccacatgtgggacgggggtagggccctactcgactggttcttggtcgtcagcctgacctgcctggtcaaaggcttctatcccagcgacatcgccgtggagtgggagcagtcggactggacggaccagtttccgaagatagggtcgctgtagcggcacctcaccctcagcaatgggcagccggagaacaactacaagaccacgcctcccgtgttggactccgacggctcgttacccgtcggcctcttgttgatgttctggtgcggagggcacaacctgaggctgccgtccttcttcctctacagcaagctcaccgtcgacaagagcaggtggcagcaggggaacgtcaggaagaaggagatgtcgttcgagtggcagctgttctcgtccaccgtcgtccccttgcagttctcatgctccgtgatgcatgaggctctgcacaaccactacacgcagaagagcctctccaagagtacgaggcactacgtactccgagacgtgttggtgatgtgcgtcttctcggagaggctgtctccgggtaaacggcgccgccggagcggtggcggcggatcaggtgggggtggatcagacagaggcccatttgccgcggcggcctcgccaccgccgcctagtccacccccacctagtggcggtggaggttccggtggcgggggatccaggaagaggaggaagagggacaaaactcacccgccacctccaaggccaccgccccctaggtccttctcctccttctccctgttttgagtgacatgcccaccgtgcccagcaccggaactcctgggcggaccgtcagtcttcctcttcccctgtacgggtggcacgggtcgtggccttgaggacccgcctggcagtcagaaggagaaggggccaaaacccaaggacaccctcatgatctcccggacccctgaggtcacatgcgtggtggtgggttttgggttcctgtgggagtactagagggcctggggactccagtgtacgcaccaccacgacgtgagccacgaagaccctgaggtcaagttcaactggtacgtggacggcgtggaggtgctgcactcggtgcttctgggactccagttcaagttgaccatgcacctgccgcacctccaccataatgccaagacaaagccgcgggaggagcagtacaacagcacgtaccgtgtggtcagcgtattacggttctgtttcggcgccctcctcgtcatgttgtcgtgcatggcacaccagtcggtcctcaccgtcctgcaccaggactggctgaatggcaaggagtacaagtgcaaggtctcccaggagtggcaggacgtggtcctgaccgacttaccgttcctcatgttcacgttccagaggaacaaagccctcccagcccccatcgagaaaaccatctccaaagccaaagggcagccccgattgtttcgggagggtcgggggtagctcttttggtagaggtttcggtttcccgtcggggctgaaccacaggtgtacaccctgcccccatcccgggatgagctgaccaagaaccaggtcagccttggtgtccacatgtgggacgggggtagggccctactcgactggttcttggtccagtcgctgacctgcctggtcaaaggcttctatcccagcgacatcgccgtggagtgggagagcaatgactggacggaccagtttccgaagatagggtcgctgtagcggcacctcaccctctcgttagggcagccggagaacaactacaagaccacgcctcccgtgttggactccgacggctccttccccgtcggcctcttgttgatgttctggtgcggagggcacaacctgaggctgccgaggaagttcctctacagcaagctcaccgtggacaagagcaggtggcagcaggggaacgtcttctcaaaggagatgtcgttcgagtggcacctgttctcgtccaccgtcgtccccttgcagaagagttgctccgtgatgcatgaggctctgcacaaccactacacgcagaagagcctctccctgtctacgaggcactacgtactccgagacgtgttggtgatgtgcgtcttctcggagagggacagaccgggtaaatgagcggccgcggcccatttactcgccggcg

SEQ ID NO: 70 - protein, 1041 aa, Artificial Sequence [sequence omitted]

SEQ ID NO: 71 - protein, 264 aa, Artificial Sequence
  GFP protein
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKSRTSGSPGLQEFDIKLIDTVDLESCN

SEQ ID NO: 72 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 74 - protein, 10 aa, Artificial Sequence
  Cysteine-containing peptide
GGGSGCGGGS

SEQ ID NO: 75 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 76 - protein, 13 aa, Artificial Sequence
  Biotin Acceptor Peptide
LNDIFEAQKIEWH

SEQ ID NO: 77 - protein, 13 aa, Artificial Sequence
  Lipoate Acceptor Peptide 2 (LAP2)
GFEIDKVWYDLDA

SEQ ID NO: 78 - protein, 5 aa, Artificial Sequence
  HAPylation motif
  REPEAT (1)..(5): Repeat from 1- 400
GGGGS

SEQ ID NO: 79-80 - skipped (2 entries)

SEQ ID NO: 81 - protein, 35 aa, Artificial Sequence
  SYN470
  MISC_FEATURE (34)..(35): PEG4
IKPEAPGEDASPEELNRYYASLRHYLNLVTRQRYG

SEQ ID NO: 82 - protein, 29 aa, Artificial Sequence
  CTP Peptide
DSSSSKAPPPSLPSPSRLPGPSDTPILPQ

SEQ ID NO: 83 - protein, 6 aa, Artificial Sequence
  FVII
IVGGKV

SEQ ID NO: 84 - protein, 11 aa, Artificial Sequence
  FVII
IVGGKVCPKGE

SEQ ID NO: 85 - protein, 6 aa, Artificial Sequence
  Factor X
IVGGQE

SEQ ID NO: 86 - protein, 11 aa, Artificial Sequence
  Factor X
IVGGQECKDGE

SEQ ID NO: 87 - protein, 99 aa, Artificial Sequence
  SUMO Protein
SLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGG

SEQ ID NO: 88 - protein, 6 aa, Artificial Sequence
  intracellular processing site
RKRRKR

SEQ ID NO: 89 - protein, 4 aa, Artificial Sequence
  intracellular processing site
RRRR

SEQ ID NO: 90 - protein, 10 aa, Artificial Sequence
  FX-PABC peptide
  MOD_RES (1)..(1): Xaa is DPhe
  MOD_RES (2)..(2): Xaa is Pip 220>
  MOD_RES (4)..(4): Xaa is P-amino benzyl carbamate
XXRXIVGGQE